(19) United States
(12) Patent Application Publication
(10) Pub. No.: US 2024/0417694 A1
(43) Pub. Date: Dec. 19, 2024

(54) MODULATING VENULENESS OF ENDOTHELIAL CELLS

(71) Applicant: President and Fellows of Harvard College, Cambridge, MA (US)

(72) Inventors: UIrich H. VON ANDRIAN, Chestnut Hill, MA (US); Aude THIRIOT, Brookline, MA (US); Kristin RATTAY, Cambridge, MA (US); Munir M. MOSAHEB, Sharon, MA (US); Rohit GARG, Boston, MA (US)

(21) Appl. No.: 18/666,005

(22) Filed: May 16, 2024

Related U.S. Application Data
(63) Continuation of application No. 16/727,131, filed on Dec. 26, 2019, now abandoned.
(60) Provisional application No. 62/785,049, filed on Dec. 26, 2018.

(51) Int. Cl. C12N 5/071; G01N 33/50

(52) U.S. Cl. CPC C12N 5/069; G01N 33/5064; C12N 2503/02

(57) ABSTRACT

Disclosed herein are genes which are differentially expressed in venule endothelial cells (V-ECs) compared to non-venule endothelial cells (NV-ECs), and methods and compositions relating to those genes. In particular, described herein are methods of reducing the venuleness of endothelial cells (ECs) for treating inflammatory diseases, and methods of increasing the venuleness of ECs for treating cancers.

### STATEMENT AS TO FEDERALLY FUNDED RESEARCH

This invention was made with government support under AI112521 and AI069259, awarded by the National Institutes of Health. The government has certain rights in the invention.

### BACKGROUND OF THE INVENTION

Inflammation is a complex biological response to a variety of noxious stimuli. Leukocyte recruitment out of blood vessels and into the inflammatory foci within the tissues is essential for mounting a successful inflammatory response to such stimuli. Appropriate levels of leukocyte recruitment and inflammation are critical for maintaining homeostasis. While higher levels of inflammation lead to pathological conditions associated with inflammatory diseases, reduced level of leukocyte recruitment is known to be associated with insufficient anti-tumor immunity in cancers. Current anti-inflammatory drugs or onco-immunotherapy drugs decrease or increase inflammation in a non-targeted manner, hence causing severe adverse effects in healthy non-target tissues. Thus, there is unmet needs in the field to develop methods for modulating (increasing or decreasing) inflammation in a tissue-specific manner so as to target the diseased tissues, while leaving the healthy tissues unharmed.

### SUMMARY OF THE INVENTION

Featured herein are methods for reducing inflammation by reducing venuleness, and increasing leukocyte recruitment by increasing venuleness of endothelial cells (ECs). Also featured are methods of treating inflammatory diseases and cancers by modulating (e.g., reducing or increasing) venuleness of ECs.

A first aspect features a method of reducing venuleness of an EC by contacting the EC with an effective amount of: (a) an agent that reduces expression of at least one gene from Tables 1-18 and/or Tables 36-52 (i.e., genes that exhibit higher expression levels in venule endothelial cells (V-ECs) compared to non-venule endothelial cells (NV-ECs)); and/or (b) an agent that increases expression of at least one gene from Tables 19-35 and/or Tables 53-68 ((i.e., genes that exhibit higher expression levels in NV-ECs compared to V-ECs). In some embodiments of this aspect, venuleness of the EC is reduced in a specific tissue (e.g., in one or more of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter).

In some embodiments, the agent that reduces expression of at least one gene exhibiting higher expression level in V-ECs compared to NV-ECs is selected from one or more of: (a) a polypeptide, such as an inhibitory antibody or antigen-binding fragment thereof directed to one or more of the genes from Tables 1-18 and/or Tables 36-52 (e.g., an inhibitory antibody or antigen-binding fragment thereof directed to one or more of the cell surface molecule genes from Tables 36-52); (b) a small molecule, such as a small molecule inhibitor; (c) a nucleic acid molecule, such as an inhibitory RNA molecule, for example, a small interfering RNA (siRNA), a short hairpin RNA (shRNA), a microRNA (miRNA), a messenger RNA (mRNA), and/or a modified mRNA (e.g., an mRNA or modified mRNA encoding an antibody and/or a protein that is directed to one or more of the genes from Tables 1-18 and/or Tables 36-52); (d) a nuclease, such as a Cas9, a transcription activator-like effector nuclease (TALEN), and/or a zinc-finger nuclease (ZFN); (e) a viral vector, such as an adeno-associated virus (AAV), an adenovirus, a parvovirus, a coronavirus, a rhabdovirus, a paramyxovirus, a picornavirus, an alphavirus, a herpes virus, a poxvirus, or a lentivirus; or (f) a plasmid, such as a plasmid encoding an antibody and/or a protein that is directed to one or more of the genes from Tables 1-18 and/or Tables 36-52.

In other embodiments, the agent that increases expression of at least one gene exhibiting higher expression level in NV-ECs compared to V-ECs is selected from one or more of (a) a polypeptide, such as an activating antibody or antigen-binding fragment thereof directed to one or more of the genes from Tables 19-35 and/or Tables 53-68 (e.g., an activating antibody or antigen-binding fragment thereof directed to one or more of the cell surface molecule genes from Tables 53-68); (b) a small molecule, such as a small molecule activator; (c) a nucleic acid molecule, such as an activating RNA molecule (e.g., a small activating RNA (saRNA), or an mRNA); (d) a viral vector, such as an AAV, an adenovirus, a parvovirus, a coronavirus, a rhabdovirus, a paramyxovirus, a picornavirus, an alphavirus, a herpes virus, a poxvirus, or a lentivirus; or (e) a plasmid, such as an overexpression plasmid.

In some embodiments, the agent is conjugated to an EC targeting molecule, which is directed to one or more genes exhibiting high expression level in ECs (e.g., one or more genes listed in Tables 69-121). In some embodiments, the EC targeting molecule is an antibody (e.g., an antibody that binds to one or more of cell surface molecule genes listed in Tables 83-96, and/or Tables 111-121). In some embodiments, conjugation to an EC targeting molecule targets the agent to an EC, such as an EC in a specific tissue (e.g., one or more of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter).

In some embodiments is disclosed a method of reducing inflammation in a tissue by reducing the venuleness of an EC, thereby treating an inflammatory disease (e.g., one or more of endotoxemia, sepsis, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischaemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasculitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Retier's syndrome, or Hodgkin's disease) in a subject (e.g., a human). In some instances, treating an inflammatory disease by the methods disclosed herein includes administration of a therapeutically effective amount of an agent that decreases inflammation by one or more of the methods described herein. Such agents may be administered systemically (e.g., intravenously), or locally.

In additional embodiments, treating an inflammatory disease by the methods disclosed herein further includes administering one or more anti-inflammatory drug (e.g., one or more of a disease-modifying anti-rheumatic drug (DMARD), a biologic response modifier (a type of DMARD), a corticosteroid, a nonsteroidal anti-inflammatory medication, prednisone, prednisolone, methylprednisolone, methotrexate, hydroxychloroquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, a nucleoside-analog reverse transcriptase inhibitor, a non-nucleoside reverse transcriptase inhibitor, an antibacterial compound, an antifungal compound, or an antiparasitic compound) to the subject, either alone or in combination with one or more of the EC targeting molecules described herein.

A second aspect features a method of increasing venuleness of an EC by contacting the EC with an effective amount of: (a) an agent that increases expression of at least one gene from Tables 1-18 and/or Tables 36-52 (i.e., genes that exhibit higher expression levels in V-ECs compared to NV-ECs); and/or (b) an agent that decreases expression of at least one gene from Tables 19-35 and/or Tables 53-68 (i.e., genes that exhibit higher expression levels in NV-ECs compared to V-ECs).

In some embodiments of the second aspect, the venuleness of the EC is increased in a specific tissue (e.g., one or more of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter).

In some embodiments of this aspect, the agent that increases expression of at least one gene exhibiting higher expression level in V-ECs compared to NV-ECs is selected from one or more of: (a) a polypeptide, such as an activating antibody or antigen-binding fragment thereof directed to one or more of the genes from Tables 1-18 and/or Tables 36-52 (e.g., an antibody or antigen-binding fragment thereof directed to one or more of the cell surface molecule genes from Tables 36-52); (b) a small molecule, such as a small molecule activator; (c) a nucleic acid molecule, such as an activating RNA molecule (e.g., an saRNA, or an mRNA); (d) a viral vector, such as an AAV, an adenovirus, a parvovirus, a coronavirus, a rhabdovirus, a paramyxovirus, a picornavirus, an alphavirus, a herpes virus, a poxvirus, or a lentivirus; or (e) a plasmid, such as an overexpression plasmid.

In other embodiments of this aspect, the agent that reduces expression of at least one gene exhibiting lower expression level in V-ECs compared to NV-ECs is selected from one or more of: (a) a polypeptide, such as an inhibitory antibody or antigen-binding fragment thereof directed to one or more of the genes from Tables 19-35 and/or Tables 53-68 (e.g., an inhibitory antibody or antigen-binding fragment directed to one or more of the cell surface molecule genes from Tables 53-68); (b) a small molecule, such as a small molecule inhibitor; (c) a nucleic acid molecule, such as an inhibitory RNA molecule, for example, an siRNA, a shRNA, a miRNA, an mRNA, and/or a modified mRNA (e.g., an mRNA or modified mRNA encoding an antibody and/or a protein that is directed to one or more of the genes from Tables 19-35 and/or Tables 53-68); (d) a nuclease, such as a Cas9, a TALEN, or ZFN; (e) a viral vector, such as an AAV, an adenovirus, a parvovirus, a coronavirus, a rhabdovirus, a paramyxovirus, a picornavirus, an alphavirus, a herpes virus, a poxvirus, and a lentivirus; or a plasmid, such as a plasmid encoding an antibody and/or a protein that is directed to one or more of the genes from Tables 19-35 and/or Tables 53-68.

In additional embodiments, the agent is conjugated to an EC targeting molecule that is directed to one or more genes exhibiting high expression level in ECs (e.g., one or more genes from Tables 69-121). In some embodiments the EC targeting molecule is an antibody (e.g., an antibody that binds to one or more of cell surface molecule genes listed in Tables 83-96, and/or Tables 111-121). In some embodiments, conjugation to an EC targeting molecule targets the agent to an EC, such as an EC of a specific tissue (e.g., one or more of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter).

Some embodiments of the second aspect feature a method of increasing leukocyte recruitment in a tissue by increasing the venuleness of an EC in the tissue according to the methods described herein. Such embodiments, additionally features a method of treating a cancer (e.g., one or more of multiple myeloma, breast cancer, acute myelogenous leukemia, acute lymphoblastic leukemia, chronic lymphocytic leukemia, myelodysplastic syndrome, chronic myelogenous leukemia—chronic phase, diffuse large B-cell lymphoma, cutaneous T-cell lymphoma, peripheral T-cell lymphoma, Hodgkin's lymphoma, hepatocellular carcinoma, cervical cancer, prostate cancer, kidney cancer, renal cell carcinoma, esophageal cancer, melanoma, glioma, pancreatic cancer, ovarian cancer, gastrointestinal stromal tumors, sarcoma, estrogen receptor-positive breast cancer, lung cancer, non-small cell lung carcinoma, mesothelioma, intestinal cancer, colon cancer, bladder cancer, adrenal cancer, gallbladder cancer, or squamous cell carcinoma of the head and neck) in a subject (e.g., a human) by increasing the venuleness of an EC according to the featured methods. In some instances, treating cancer by one or more of the methods described herein include administration of a therapeutically effective amount of an agent that increases leukocyte recruitment by one or more of the methods described herein. Such agents may be administered systemically (e.g., intravenously), or locally (e.g., intratumorally). In some embodiments, such methods also include administration of one or more anti-cancer therapeutics to the subject, either alone, or in combination with one or more of the EC targeting molecules. Such anti-cancer therapeutics include: (a) one or more chemotherapeutics, such as an anthracycline (e.g., doxorubicin), a nucleoside analog (e.g., fluorouracil), a platinum-based anti-neoplastic agent (e.g., cisplatin), a taxane (e.g., paclitaxel), a vinca alkaloid (e.g., vincristine), a glycopeptide antibiotic (e.g., bleomycin), or a polypeptide antibiotic (e.g., actinomycin D); (b) one or more targeted anti-cancer therapeutic, such as a tyrosine kinase inhibitor, a PI3K inhibitor, a multi-kinase inhibitor, a CDK4/6 inhibitor, an mTOR inhibitor, a NOTCH inhibitor, an HSP90 inhibitor, an HSP70 inhibitor, a proteasome inhibitor, or a tumor metabolism inhibitor; and/or (c) one or more anti-cancer immunotherapeutic, such as a cytokine (e.g., IL-2, IFN-alpha), a monoclonal antibody, an immune checkpoint inhibitor (e.g., PD-1 inhibitor, PD-L1 inhibitor, CTLA-4 inhibitor), an inhibitor of the Carma1-BCL10-MALT-1 complex, a cancer vaccine, a tumor-infiltrating lymphocyte (TIL), a CAR T-cell, or a non-specific immunotherapeutic.

### Definitions

As used herein, the term “venule” refers to a small blood vessel in the microcirculation that allows deoxygenated blood to return from capillary beds to larger blood vessels called veins. Venules range from 7 μm to 1 mm in diameter, and are formed when capillaries come together. Many venules unite to form a vein. The wall of a venule is made up of three layers: an inner endothelium composed of ECs that acts as a membrane; a middle layer of muscle and elastic tissue; and an outer layer of fibrous connective tissue. The middle layer is poorly developed so that venules have thinner walls than arterioles. Venules are porous so that fluid and blood cells can easily move from the bloodstream through their walls. Venules are the only microvessels that have the capacity to recruit inflammatory leukocytes from blood into tissues (e.g., leukocyte trafficking through post-capillary venules or collecting venules), and thus actively regulate inflammation.

As used herein, “venule ECs,” “V-ECs” and “venular ECs” are used interchangeably to refer to ECs that line the inner lumina or wall, and form the endothelium of venules. It should be appreciated that a venule or venule EC (V-EC) may display a marker or combination of markers indicative of venuleness (e.g., a gene or combination of genes which is differentially or selectively expressed in venule ECs compared to non-venule ECs (NV-ECs)). V-ECs of mesenteric lymph node, Peyer's patch, small intestine, and colon are referred to herein as “gut V-ECs.”

As used herein, the term “venuleness” refers to one or more properties of venules. Venuleness includes the physical and functional properties that distinguish venules from non-venules. Alternatively, “venuleness” may also refer to one or more physical and functional properties of V-ECs that distinguish V-ECs from NV-ECs. It should be appreciated that a V-EC may display a marker or combination of markers indicative of venuleness (e.g., a gene or combination of genes that is differentially or selectively expressed or has higher expression level in V-ECs compared to NV-ECs). Also, expression of a non-signaling chemokine binding receptor, DARC (Duffy Antigen/Receptor for Chemokines, a.k.a. ACKR1 or CD234), which had been suggested to be a specific marker for V-ECs in humans, can be considered as an indicative feature of venuleness. Examples of venuleness may also include leukocyte recruitment and regulation of inflammation, functional attributes that are unique to venules and/or V-ECs, and not shared by the non-venules or NV-ECs.

As used herein, the term “non-venule” refers to any microvessel that is not a venule. Non-venules do not exhibit venuleness. Unlike venules, leukocyte trafficking does not occur through non-venules, and thus non-venules do not have a role in regulation of inflammation. Examples of non-venules include arterioles and capillaries.

As used herein, “non-venule ECs”, “NV-ECs” and “non-venular ECs” are used interchangeably to refer to ECs that line the inner lumina or wall, and form the endothelium of non-venules. It should be appreciated that a non-venule or NV-EC may display a marker or combination of markers indicative of non-venuleness (e.g., a gene or combination of genes which is differentially or selectively expressed or has higher expression level in NV-ECs compared to V-ECs). Alternatively, absence of venuleness (e.g., absence of one or more physical and functional indications of venuleness, such as absence of venuleness markers (e.g., absence of DARC expression)) may also be considered as an indication of non-venuleness.

As used herein, “high endothelial venule” or “HEV” refers to a blood vessel that is especially adapted for recruitment of lymphocytes. HEVs are normally found in secondary lymphoid organs such as lymph nodes (e.g., mesenteric lymph nodes and peripheral lymph nodes) and Peyer's patches, and form a spatially organized network of blood vessels, which controls both the type of lymphocyte and the site of entry into lymphoid tissues. HEVs are characterized by high ECs (also known as plump ECs) as opposed to the usual thin or flat ECs found in regular venules. Another defining feature of HEVs is the presence of lymphocytes within the EC lining and the surrounding basal lamina that illustrates the function of HEVs in lymphocyte recruitment, and explains why these vessels were implicated in lymphocyte trafficking from the time of their initial description. Vessels with HEV phenotype appear in human tissue in association with long-standing chronic inflammation, such as in rheumatoid arthritis and inflammatory bowel diseases. HEVs are also found in tumors, and their presence correlates with reduced tumor size and improved patient outcome. Newly formed HEVs promote anti-cancer immunity by increasing lymphocyte recruitment into the tumor.

As used herein, “HEV phenotype” refers to one or more properties of HEVs. HEV phenotype includes the physical and functional properties that distinguish HEVs from non-venules and other regular venules. Alternatively, “HEV phenotype” may also refer to one or more physical and functional properties of high ECs that distinguish high ECs from other ECs. It should be appreciated that a high EC may display a marker or combination of markers indicative of HEV phenotype (e.g., a gene or combination of genes that is differentially or selectively expressed or has higher expression level in V-ECs in mesenteric lymph nodes, peripheral lymph nodes, and Peyer's patches compared to NV-ECs). Examples of HEV phenotype may include lymphocyte recruitment and regulation of chronic inflammation, functional attributes that are unique to HEVs, and not shared by the non-venules or other regular venules.

As used interchangeably herein, “leukocyte migration”, “leukocyte trafficking” and “leukocyte recruitment” refer to the movement or migration of leukocytes out of the circulatory system and towards the site of tissue damage, infection, injury, or stress. Leukocyte recruitment from the bloodstream to the inflammatory foci within the tissue is fundamental to mounting a successful inflammatory response, and forms an essential part of the innate immune response, as evidenced by the recurrent infections and poor survival rate of patients suffering from leukocyte adhesion deficiencies, a class of conditions in which neutrophil trafficking is compromised. Monocytes also use this process in the absence of infection or tissue damage during their development into macrophages. Leukocyte recruitment occurs mainly in post-capillary venules, where molecules that regulate leukocyte trafficking are preferentially expressed. During the process of leukocyte recruitment, leukocytes adhere to the vascular endothelium, and subsequently leave the circulation by transendothelial migration driven by chemoattractants (e.g., chemokines), a process known as diapedesis.

As used herein, “inflammation” refers to a signal-mediated response to cellular insult by infectious agents (e.g., pathogens), toxins, tumor cells, irritants and stress. While acute inflammation is important to the defense and protection of body from harmful stimuli (e.g., pathogens, damaged cells, cancer/tumor cells, stress, or irritants), chronic and inappropriately high inflammation can cause tissue destruction (e.g., in autoimmunity, inflammatory diseases, neurodegenerative diseases, or cardiovascular disease). Inflammation represents the consequence of capillary dilation with accumulation of fluid (edema) and the recruitment of leukocytes. For the purpose of use herein, increase or decrease in inflammation is assessed by increase or decrease of leukocyte recruitment, and/or increase or decrease of immune cell activity (e.g., one or more of T cell polarization; T cell activation; dendritic cell activation; neutrophil activation; eosinophil activation; basophil activation; T cell proliferation; B cell proliferation; monocyte proliferation; macrophage proliferation; dendritic cell proliferation; NK cell proliferation; ILC proliferation, mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation, ILC ADCC, ILC degranulation, cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of HEVs; or development of ectopic or tertiary lymphoid organs (TLOs)).

As used herein, the terms “increase” or “increasing” and “decrease” or “decreasing” refer to modulating resulting in, respectively, greater or lesser amounts, of function, expression level, or activity of a metric relative to a reference. For example, subsequent to administration of a one or more the agents in the methods described herein, the expression level of one or more the genes listed in the Tables herein may be increased or decreased in a subject by at least 5%, 10%, 15%, 20%, 25%, 30%, 35%, 40%, 45%, 50%, 55%, 60%, 65%, 70%, 75%, 80%, 85%, 90%, 95% or 98% or more relative to expression level of the gene prior to administration of the agent. Generally, the metric (e.g., expression level) is measured subsequent to administration at a time that the administration has had the recited effect, e.g., at least one week, one month, 3 months, or 6 months, after a treatment regimen has begun. The term “reducing” is used interchangeably with the term “decreasing” herein.

As used herein, the terms “higher” and “lower” refer to respectively, greater or lesser amounts, of expression level, function, or activity of a metric relative to a reference. For example, expression level of a gene is higher in V-ECs compared to NV-ECs mean that expression level of that gene in V-ECs is greater than that in NV-ECs by at least ≥1.2 fold, ≥1.3 fold, ≥1.4 fold, ≥1.5 fold, 21.6 fold, 21.7 fold, 21.8 fold, ≥1.9 fold, ≥2.0 fold, or more.

As used herein, the term “bone tissue” refers to bone and/or bone marrow. For example, expression of a gene is higher in V-ECs compared to NV-ECs in bone tissue means that expression of the gene is higher in V-ECs compared to NV-ECs in bone and/or in bone marrow.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, inhalation routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “effective amount,” “therapeutically effective amount,” and a “sufficient amount” of a composition, antibody, vector construct, viral vector or cell described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. For example, in the context of treating inflammatory or autoimmune disease or condition it is an amount of the composition, antibody, vector construct, viral vector or cell sufficient to achieve a treatment response as compared to the response obtained without administration of the composition, antibody, vector construct, viral vector or cell. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art. Also, as used herein, a “therapeutically effective amount” of a composition, antibody, vector construct, viral vector or cell of the present disclosure is an amount that results in a beneficial or desired result in a subject as compared to a control. As defined herein, a therapeutically effective amount of a composition, antibody, vector construct, viral vector or cell of the present disclosure may be readily determined by one of ordinary skill by routine methods known in the art. Dosage regimen may be adjusted to provide the optimum therapeutic response.

As used herein, “locally” or “local administration” means administration at a particular site of the body intended for a local effect and not a systemic effect. Examples of local administration are epicutaneous, inhalational, intra-articular, intrathecal, intravaginal, intravitreal, intrauterine, intralesional administration, lymph node administration, intratumoral administration and administration to a mucous membrane of the subject, wherein the administration is intended to have a local and not a systemic effect.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “proliferation” refers to an increase in cell numbers through growth and division of cells.

As used herein, the term “reference” refers to a level, expression level, copy number, sample or standard that is used for comparison purposes. For example, a reference sample can be obtained from a healthy individual (e.g., an individual who does not have cancer). A reference level can be the level of expression of one or more reference samples. For example, an average expression (e.g., a mean expression or median expression) among a plurality of individuals (e.g., healthy individuals, or individuals who do not have cancer). In other instances, a reference level can be a predetermined threshold level, e.g., based on functional expression as otherwise determined, e.g., by empirical assays.

As used herein, the term “sample” refers to a specimen (e.g., blood, blood component (e.g., serum or plasma), urine, saliva, amniotic fluid, cerebrospinal fluid, tissue (e.g., barrier tissue, skin, gut tissue, airway tissue, wound tissue, placental, or dermal), pancreatic fluid, chorionic villus sample, and cells) isolated from a subject.

As used herein, the terms “subject” and “patient” refer to an animal (e.g., a mammal, such as a human). A subject to be treated according to the methods described herein may be one who has been diagnosed with a particular condition, or one at risk of developing such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

“Treatment” and “treating,” as used herein, refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “activation” refers to the response of an immune cell to a perceived insult. When immune cells become activated, they proliferate, secrete cytokines, differentiate, present antigens, become more polarized, and can become more phagocytic and cytotoxic. Factors that stimulate immune cell activation include pro-inflammatory cytokines, pathogens, and non-self antigen presentation (e.g., antigens from pathogens presented by dendritic cells, macrophages, or B cells).

As used herein, the term “about” refers to a value that is ±10% of the recited value.

As used herein, the term “cancer” refers to a condition characterized by unregulated or abnormal cell growth. The terms “cancer cell,” “tumor cell,” and “tumor” refer to an abnormal cell, mass, or population of cells that result from excessive division that may be malignant or benign and all pre-cancerous and cancerous cells and tissues.

Throughout this specification and claims, the word “comprise,” or variations such as “comprises” or “comprising,” will be understood to imply the inclusion of a stated integer or group of integers but not the exclusion of any other integer or group of integers.

As used herein, the term “analog” refers to a protein of similar nucleotide or amino acid composition or sequence to any of the proteins or peptides, allowing for variations that do not have an adverse effect on the ability of the protein or peptide to carry out its normal function. Analogs may be the same length, shorter, or longer than their corresponding protein or polypeptide. Analogs may have about 60% (e.g., about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 99%) identity to the amino acid sequence of the naturally occurring protein or peptide. An analog can be a naturally occurring protein or polypeptide sequence that is modified by deletion, addition, mutation, or substitution of one or more amino acid residues.

As used herein, the term “antibody” refers to a molecule that specifically binds to, or is immunologically reactive with, a particular antigen and includes at least the variable domain of a heavy chain, and normally includes at least the variable domains of a heavy chain and of a light chain of an immunoglobulin. Antibodies and antigen-binding fragments, variants, or derivatives thereof include, but are not limited to, polyclonal, monoclonal, multispecific, human, humanized, primatized, or chimeric antibodies, heteroconjugate antibodies (e.g., bi- tri- and quad-specific antibodies, diabodies, triabodies, and tetrabodies), single-domain antibodies (sdAb), epitope-binding fragments, e.g., Fab, Fab′ and F(ab′)₂, Fd, Fvs, single-chain Fvs (scFv), rIgG, single-chain antibodies, disulfide-linked Fvs (sdFv), fragments including either a V_{L} or V_{H} domain, fragments produced by an Fab expression library, and anti-idiotypic (anti-Id) antibodies. Antibody molecules of the invention can be of any type (e.g., IgG, IgE, IgM, IgD, IgA, and IgY), class (e.g., IgG1, IgG2, IgG3, IgG4, IgA1 and IgA2) or subclass of immunoglobulin molecule. Moreover, unless otherwise indicated, the term “monoclonal antibody” (mAb) is meant to include both intact molecules as well as antibody fragments (such as, for example, Fab and F (ab′)₂ fragments) that are capable of specifically binding to a target protein. Fab and F (ab′)₂ fragments lack the Fc fragment of an intact antibody.

The term “antigen-binding fragment,” as used herein, refers to one or more fragments of an immunoglobulin that retain the ability to specifically bind to a target antigen. The antigen-binding function of an immunoglobulin can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed by the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂ fragment, a bivalent fragment containing two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb (Ward et al., Nature 341:544-546, 1989) including V_{H} and V_{L} domains; (vi) a dAb fragment that consists of a V_{H} domain; (vii) a dAb that consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv)). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “cell type” refers to a group of cells sharing a phenotype that is statistically separable based on gene expression data. For instance, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, barrier tissue, mucosal tissue, gut, or muscle tissue) and/or those that are isolated from a common organ, tissue system, blood vessel, or other structure and/or region in an organism.

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

Other features and advantages of the invention will be apparent from the following Detailed Description and the claims.

### BRIEF DESCRIPTION OF THE DRAWINGS

FIG. 1 is an immunohistochemistry micrograph of a whole mount staining in mouse omentum showing staining of venules with anti-DARC Ab (left panel) and the microvasculature with anti-CD31 Ab (middle panel). Arrows on the merged image (right panel) indicate the localization of arteriole, capillaries and venules. DARC staining is seen only in postcapillary and small collecting venules.

FIG. 2 is a schematic representation of the global prospects of the featured invention.

### DETAILED DESCRIPTION

Disclosed herein are methods of reducing the venuleness of endothelial cells (ECs) for treating inflammatory diseases, and methods of increasing the venuleness of ECs for treating cancers. Also described are genes that are differentially expressed in venule ECs (V-ECs) compared to non-venule ECs (NV-ECs) either globally (e.g., in all tissues) or in a tissue-specific manner (e.g., in colon), and methods of reducing or increasing venuleness of ECs by regulating those genes. These methods provide new therapeutic approaches for treating inflammatory diseases and cancers in a targeted way (e.g., in a specific tissue). Thus, the featured methods contemplate new generation of anti-inflammatory and immuno-oncotherapy drugs that specifically target venular endothelium, either globally or exclusively in a selected tissue for a variety of pathologies. This strategy is in stark contrast to other currently prevalent strategies that are largely based on systemic administration of drugs. One of the key anticipated benefits of the featured methods is the reduction of adverse side effects in uninvolved tissue during treatment of disease.

### I. Venule and Non-venule ECs

Vascular ECs (ECs) form the inner lining of all blood vessels from the largest artery and veins, namely, the aorta and venae cavae, respectively, to the capillaries that connect the arterial and venous systems. Because these two major conducting systems of the vasculature differ functionally, it is not surprising that the physical makeup of arteries and veins, including the ECs that line their lumina, are also distinct. ECs lining venules and non-venules also differ functionally. Venule ECs (V-ECs) that line inner wall of venules, the primary site of leukocyte recruitment, play a major role in regulating inflammation. Contrarily, non-venule ECs (NV-ECs) that line the inner lumina of non-venules (e.g., capillaries and arterioles) are not involved in such functions, and primarily regulate vascular tone, and nutrient and gas exchange. Such distinct properties of V-ECs and NV-ECs are determined and regulated by the genetic makeup of the cells.

Identified herein are genes that are selectively expressed in V-ECs or NV-ECs either globally (e.g., in all tissues or in a large number of tissues) or in a tissue-restricted fashion (e.g., in a specific tissue, such as colon). Identification of such segmental difference in microvascular gene expression is important as the molecular mechanisms that govern venular function are likely to be rooted in venule-specific gene expression. Genes that are selectively expressed in V-ECs or NV-ECs (i.e., have higher expression level in V-ECs compared to NV-ECs, or higher expression level in NV-ECs compared to V-ECs) determine the venuleness or non-venuleness of ECs, and can be modulated (e.g., increased or decreased) to modulate (e.g., increase or decrease) venuleness. Also identified are HEV-specific genes that are selectively expressed in V-ECs in mesenteric lymph node, peripheral lymph node and Peyer's patch, but not in other tissues, i.e., genes that are selectively expressed in HEVs. The HEV-specific genes determine the HEV phenotype, and can be modulated (e.g., increased or decreased) to modulate (e.g., increase or decrease) the HEV phenotype.

Increase of venuleness by increase in expression level of V-EC-specific genes or decrease in expression level of NV-EC specific genes may induce venular differentiation, leading to an increase in venular functions and venule-specific functional properties of ECs. A plausible outcome of such increase in venuleness and venular differentiation is increased leukocyte recruitment, which may have a therapeutic benefit in cancer treatment by promoting anti-tumor immunity. Alternatively, decrease of venuleness by decrease in expression level of V-EC-specific genes or increase in expression level of NV-EC-specific genes may inhibit venular differentiation, leading to a decrease in venular functions and venule-specific functional properties of ECs. A plausible outcome of such decrease in venuleness and venular differentiation is decreased or reduced leukocyte recruitment, and/or decreased or reduced inflammation, which may have a therapeutic benefit in treatment of inflammatory and autoimmune diseases. Additionally, identification of tissue-restricted expression pattern of V-EC- or NV-EC-specific genes also allow modulation (e.g., increase or decrease) of venuleness and venular differentiation in specific tissues, thus restricting its physiologic effect (e.g., increased leukocyte recruitment or decreased inflammation) to that specific tissue only. Also, identification of genes that are selectively expressed in V-ECs in mesenteric lymph node, peripheral lymph node and Peyer's patch, but not in other tissues, i.e., identification of HEV-specific genes, allows modification (e.g., increase or decrease) of the HEV phenotype.

### II. Modulating the Venuleness of ECs

Featured herein are methods for modulating (e.g., increasing or decreasing) venuleness by modulating (e.g., increasing or decreasing) the expression of genes that are selectively expressed on V-ECs or NV-ECs.

### Reducing Venuleness of ECs

Venuleness of ECs may be reduced by one or more of the methods described herein. In some embodiments, venuleness of an EC may be reduced by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥2 20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs. Such a gene may be selected from one or more of the genes listed in Tables 1-18 (e.g., Table 1, Table 2, Table 3, Table 4, Table 5, Table 6, Table 7, Table 8, Table 9, Table 10, Table 11, Table 12, Table 13, Table 14, Table 15, Table 16, Table 17, and/or Table 18), and/or Tables 36-52 (e.g., Table 36, Table 37, Table 38, Table 39, Table 40, Table 41, Table 42, Table 43, Table 44, Table 45, Table 46, Table 47, Table 48, Table 49, Table 50, Table 51, and/or Table 52). The expression level of the at least one gene that has higher expression level in V-ECs compared to NV-ECs can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC. For example, the expression level of at least one cell surface molecule from Tables 36-52 that has higher expression level in V-ECs compared to NV-ECs can be reduced by contacting the EC with an inhibitory antibody directed against that cell surface molecule.

Alternatively, venuleness of an EC may be reduced by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs. Such a gene may be selected from one or more of the genes listed in Tables 19-35 (e.g., Table 19, Table 20, Table 21, Table 22, Table 23, Table 24, Table 25, Table 26, Table 27, Table 28, Table 29, Table 30, Table 31, Table 32, Table 33, Table 34, and/or Table 35), and/or Tables 53-68 (e.g., Table 53, Table 54, Table 55, Table 56, Table 57, Table 58, Table 59, Table 60, Table 61, Table 62, Table 63, Table 64, Table 65, Table 66, Table 67, and/or Table 68). The expression level of the at least one gene that has higher expression level in NV-ECs compared to V-ECs can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level and/or induces the activity of the gene in the EC. For example, the expression level of at least one cell surface molecule from Tables 53-68 that has higher expression level in NV-ECs compared to V-ECs can be increased by contacting the EC with an activating antibody directed against that cell surface molecule.

### Reducing Venuleness of ECs in a Tissue-Specific Manner

Venuleness of ECs may be reduced in a tissue-specific manner (e.g., in a specific tissue) by one or more of the methods described herein. In some embodiments, venuleness of an EC may be reduced in a specific tissue (e.g., peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in that specific tissue. The expression level of the at least one gene that has higher expression level in V-ECs compared to NV-ECs in the specific tissue can be reduced by contacting the EC with one or more (e.g., 1, 2, 3, 4, 5, 6, 7, 8, 9, 10, or more) agent (e.g., a polypeptide (e.g., an antibody), a small molecule (e.g., a small molecule inhibitor), a nucleic acid molecule (e.g., an inhibitory RNA, such as an siRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in that specific tissue.

In specific embodiments:
(a) venuleness of an EC may be reduced in peripheral lymph node by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in peripheral lymph node; such a gene may be selected from one or more of the genes listed in Table 4 and/or Table 39; or
(b) venuleness of an EC may be reduced in mesenteric lymph node by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in mesenteric lymph node; such a gene may be selected from one or more of the genes listed in Table 5 and/or Table 40; or
(c) venuleness of an EC may be reduced in Peyer's patch by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in Peyer's patch; such a gene may be selected from one or more of the genes listed in Table 6 and/or Table 41; or
(d) venuleness of an EC may be reduced in thymus by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in thymus; such a gene may be selected from one or more of the genes listed in Table 7 and/or Table 42; or
(e) venuleness of an EC may be reduced in visceral adipose tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in visceral adipose tissue; such a gene may be selected from one or more of the genes listed in Table 8 and/or Table 43; or
(f) venuleness of an EC may be reduced in small intestine by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in small intestine; such a gene may be selected from one or more of the genes listed in Table 9 and/or Table 44; or
(g) venuleness of an EC may be reduced in colon by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in colon; such a gene may be selected from one or more of the genes listed in Table 10 and/or Table 45; or
(h) venuleness of an EC may be reduced in subcutaneous adipose tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in subcutaneous adipose tissue; such a gene may be selected from one or more of the genes listed in Table 11 and/or Table 46; or
(i) venuleness of an EC may be reduced in bone tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in bone tissue; such a gene may be selected from one or more of the genes listed in Table 12 and/or Table 47.
(j) venuleness of an EC may be reduced in uterine tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in uterine tissue; such a gene may be selected from one or more of the genes listed in Table 13 and/or Table 48.
(k) venuleness of an EC may be reduced in brain parenchyma by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in brain parenchyma; such a gene may be selected from one or more of the genes listed in Table 14 and/or Table 49.
(l) venuleness of an EC may be reduced in pia matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in pia matter; such a gene may be selected from one or more of the genes listed in Table 15 and/or Table 50.
(m) venuleness of an EC may be reduced in dura matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in dura matter; such a gene may be selected from one or more of the genes listed in Table 16 and/or Table 51.

Alternatively, venuleness of an EC may be reduced in a tissue-specific manner (e.g., in a specific tissue) by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs in that specific tissue. The expression level of the at least one gene that has higher expression level in NV-ECs compared to V-ECs in the specific tissue can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level of the gene in the EC in that specific tissue.

In specific embodiments:
(a) venuleness of an EC may be reduced in peripheral lymph node by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in peripheral lymph node; such a gene may be selected from one or more of the genes listed in Table 22 and/or Table 56; or
(b) venuleness of an EC may be reduced in mesenteric lymph node by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in mesenteric lymph node; such a gene may be selected from one or more of the genes listed in Table 23 and/or Table 57; or
(c) venuleness of an EC may be reduced in Peyer's patch by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in Peyer's patch; such a gene may be selected from one or more of the genes listed in Table 24 and/or Table 58; or
(d) venuleness of an EC may be reduced in thymus by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in thymus; such a gene may be selected from one or more of the genes listed in Table 25 and/or Table 59; or
(e) venuleness of an EC may be reduced in visceral adipose tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in visceral adipose tissue; such a gene may be selected from one or more of the genes listed in Table 26 and/or Table 60; or
(f) venuleness of an EC may be reduced in small intestine by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in small intestine; such a gene may be selected from one or more of the genes listed in Table 27 and/or Table 61; or
(g) venuleness of an EC may be reduced in colon by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in colon; such a gene may be selected from one or more of the genes listed in Table 28 and/or Table 62; or
(h) venuleness of an EC may be reduced in subcutaneous adipose tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in subcutaneous adipose tissue; such a gene may be selected from one or more of the genes listed in Table 29 and/or Table 63; or
(i) venuleness of an EC may be reduced in bone tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in bone tissue; such a gene may be selected from one or more of the genes listed in Table 30 and/or Table 64.
(j) venuleness of an EC may be reduced in uterine tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in uterine tissue; such a gene may be selected from one or more of the genes listed in Table 31 and/or Table 65.
(k) venuleness of an EC may be reduced in brain parenchyma by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in brain parenchyma; such a gene may be selected from one or more of the genes listed in Table 32 and/or Table 66.
(l) venuleness of an EC may be reduced in choroid plexus by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in bone tissue; such a gene may be selected from one or more of the genes listed in Table 33 and/or Table 67.
(m) venuleness of an EC may be reduced in pia matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in pia matter; such a gene may be selected from one or more of the genes listed in Table 34 and/or Table 68.
(n) venuleness of an EC may be reduced in dura matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in dura matter; such a gene may be selected from one or more of the genes listed in Table 35.

### Reducing Venuleness of ECs in Multiple Tissues

Venuleness of ECs may be reduced in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by one or more of the methods described herein. In some embodiments, venuleness of an EC may be reduced in multiple tissues by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in those multiple tissues. The expression level of the at least one gene that has higher expression level in V-ECs compared to NV-ECs in multiple tissues can be reduced by contacting the EC with one or more (e.g., 1, 2, 3, 4, 5, 6, 7, 8, 9, 10, or more) agent (e.g., a polypeptide (e.g., an antibody), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in those multiple tissues.

In specific embodiments, venuleness of an EC may be reduced in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Such a gene may be selected from one or more of the genes listed in Table 1, Table 2, Table 3, Table 36, Table 37, and/or Table 38. The expression level of the at least one gene that has higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in all of peripheral lymph node, mesenteric lymph node, Peyer's patch, thymus, visceral adipose tissue, small intestine, colon, subcutaneous adipose tissue, and bone tissue. For example, the expression level of at least one cell surface molecule from Table 36, Table 37, and/or Table 38 that has higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be reduced by contacting the EC with an inhibitory antibody directed against that cell surface molecule.

Alternatively, venuleness of an EC may be reduced in multiple (e.g., in more than one, such as in 2, 3, 4, 5, 6, 7, 8, 9, 10, 11, 12, 13 or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs in those multiple tissues. The expression level of the at least one gene that has higher expression level in NV-ECs compared to V-ECs in multiple tissues can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level and/or induces the activity of the gene in the EC in those multiple tissues. For example, the expression level of at least one gene from Tables 19-35 and/or Tables 53-68 that has higher expression level in NV-ECs compared to V-ECs can be increased by contacting the EC with an activating antibody directed against that gene.

In specific embodiments, venuleness of an EC may be reduced in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Such a gene may be selected from one or more of the genes listed in Table 19, Table 20, Table 21, Table 53 Table 54, and/or Table 55. The expression level of the at least one gene that has higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be reduced by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level and/or induces the activity of the gene in the EC in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. For example, the expression level of at least one cell surface molecule from Table 53, Table 54, and/or Table 55 that has higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be increased by contacting the EC with an activating antibody directed against that cell surface molecule.

In additional embodiments, venuleness of an EC may be reduced in all of peripheral lymph node, mesenteric lymph node, and Peyer's patch by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in all of peripheral lymph node, mesenteric lymph node, and Peyer's patch, but not in other tissues (i.e., gene that has higher expression level in HEVs, such as HEV-specific gene). Such a gene may be selected from one or more of the genes listed in Table 17 and/or Table 52. The expression level of the at least one gene that has higher expression level in V-ECs compared to NV-ECs in all of peripheral lymph node, mesenteric lymph node, and Peyer's patch, but not in other tissues (i.e., gene that has higher expression level in HEVs, such as HEV-specific gene) can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in all of peripheral lymph node, mesenteric lymph node, and Peyer's patch. For example, the expression level of at least one cell surface molecule from Table 52 that has higher expression level in V-ECs compared to NV-ECs in all of peripheral lymph node, mesenteric lymph node, and Peyer's patch can be reduced by contacting the EC with an inhibitory antibody directed against that cell surface molecule.

In additional embodiments, venuleness of an EC may be reduced in all of mesenteric lymph node, Peyer's patch, small intestine, and colon by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in all of mesenteric lymph node, Peyer's patch, small intestine, and colon, but not in other tissues (i.e., gene that has higher expression level in gut V-ECs, such as gut V-EC specific gene). Such a gene may be the gene listed in Table 18. The expression level of the at least one gene that has higher expression level in V-ECs compared to NV-ECs in all of mesenteric lymph node, Peyer's patch, small intestine, and colon, but not in other tissues (i.e., gene that has higher expression level in gut V-ECs, such as gut V-EC specific gene) can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in all of mesenteric lymph node, Peyer's patch, small intestine, and colon. Reducing or decreasing venuleness by one or more methods described herein (e.g., decrease in expression level of V-EC-specific genes or increase in expression level of NV-EC specific genes) may also inhibit venular differentiation (i.e., inhibit differentiation of ECs into V-ECs), leading to a decrease in venular functions (e.g., leukocyte recruitment), venular phenotypes (e.g., HEV phenotype, such as decrease in HEV phenotype by decreasing the expression level of HEV-specific genes) and venule-specific functional properties of ECs. In specific embodiments, decrease in expression level of HEV-specific genes, i.e., genes that have higher expression level in V-ECs compared to NV-ECs in all of peripheral lymph node, mesenteric lymph node, and Peyer's patch, but not in other tissues, may decrease HEV phenotype. A plausible outcome of such decrease in venuleness, venular differentiation and HEV phenotype is decreased inflammation, which may have a therapeutic benefit in treatment of inflammatory and autoimmune diseases. Additionally, decrease of venuleness and venular differentiation in specific tissues may restrict its physiologic effect (e.g., decreased inflammation) to that specific tissue, thus reducing adverse effects associated with anti-inflammatory drugs that work in a non-targeted manner.

### Increasing Venuleness of ECs

Venuleness of ECs may be increased by one or more of the methods described herein. In some embodiments, venuleness of an EC may be increased by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs. Such a gene may be selected from one or more of the genes listed in Tables 1-18 (e.g., Table 1, Table 2, Table 3, Table 4, Table 5, Table 6, Table 7, Table 8, Table 9, Table 10, Table 11, Table 12, Table 13, Table 14, Table 15, Table 16, Table 17, and/or Table 18), and/or Tables 36-52 (e.g., Table 36, Table 37, Table 38, Table 39, Table 40, Table 41, Table 42, Table 43, Table 44, Table 45, Table 46, Table 47, Table 48, Table 49, Table 50, Table 51, and/or Table 52). The expression level of the at least one gene that has higher expression level in V-ECs compared to NV-ECs can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level and/or induces the activity of the gene in the EC. For example, the expression level of at least one gene from Tables 1-18 and/or Tables 36-52 that has higher expression level in V-ECs compared to NV-ECs can be increased by contacting the EC with an activating antibody directed against that gene.

Alternatively, venuleness of an EC may be increased by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs. Such a gene may be selected from one or more of the genes listed in Tables 19-35 (e.g., Table 14, Table 15, Table 16, Table 17, Table 18, Table 19, Table 20, Table 21, Table 22, Table 23, and/or Table 24), and/or Tables 53-68 (e.g., Table 53, Table 54, Table 55, Table 56, Table 57, Table 58, Table 59, Table 60, Table 61, Table 62, Table 63, Table 64, Table 65, Table 66, Table 67, and/or Table 68). The expression level of the at least one gene that has higher expression level in NV-ECs compared to V-ECs can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC. For example, the expression level of at least one gene from Tables 19-35 and/or Tables 53-68 that has higher expression level in NV-ECs compared to V-ECs can be reduced by contacting the EC with an inhibitory antibody directed against that gene.

### Increasing Venuleness of ECs in a Tissue-Specific Manner

Venuleness of ECs may be increased in a tissue-specific manner (e.g., in a specific tissue) by one or more of the methods described herein. In some embodiments, venuleness of an EC may be increased in a specific tissue (e.g., peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2 2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in that specific tissue. The expression level of the at least one gene that has higher expression level in V-ECs compared to NV-ECs in the specific tissue can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level of the gene in the EC in that specific tissue.

In specific embodiments:
(a) venuleness of an EC may be increased in peripheral lymph node by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in peripheral lymph node; such a gene may be selected from one or more of the genes listed in Table 4 and/or Table 39; or
(b) venuleness of an EC may be increased in mesenteric lymph node by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in mesenteric lymph node; such a gene may be selected from one or more of the genes listed in Table 5 and/or Table 40; or
(c) venuleness of an EC may be increased in Peyer's patch by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in Peyer's patch; such a gene may be selected from one or more of the genes listed in Table 6 and/or Table 41; or
(d) venuleness of an EC may be increased in thymus by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in thymus; such a gene may be selected from one or more of the genes listed in Table 7 and/or Table 42; or
(e) venuleness of an EC may be increased in visceral adipose tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in visceral adipose tissue; such a gene may be selected from one or more of the genes listed in Table 8 and/or Table 43; or
(f) venuleness of an EC may be increased in small intestine by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in small intestine; such a gene may be selected from one or more of the genes listed in Table 9 and/or Table 44; or
(g) venuleness of an EC may be increased in colon by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in colon; such a gene may be selected from one or more of the genes listed in Table 10 and/or Table 45; or
(h) venuleness of an EC may be increased in subcutaneous adipose tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in subcutaneous adipose tissue; such a gene may be selected from one or more of the genes listed in Table 11 and/or Table 46; or
(i) venuleness of an EC may be increased in bone tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in bone tissue; such a gene may be selected from one or more of the genes listed in Table 12 and/or Table 47.
(j) venuleness of an EC may be increased in uterine tissue by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in uterine tissue; such a gene may be selected from one or more of the genes listed in Table 13 and/or Table 48.
(k) venuleness of an EC may be increased in brain parenchyma by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in brain parenchyma; such a gene may be selected from one or more of the genes listed in Table 14 and/or Table 49.
(l) venuleness of an EC may be increased in pia matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in pia matter; such a gene may be selected from one or more of the genes listed in Table 15 and/or Table 50.
(m) venuleness of an EC may be increased in dura matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in V-ECs compared to NV-ECs in dura matter; such a gene may be selected from one or more of the genes listed in Table 16 and/or Table 51.

Alternatively, venuleness of an EC may be increased in a tissue-specific manner (e.g., in a specific tissue) by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs in that specific tissue. The expression level of the at least one gene that has higher expression level in NV-ECs compared to V-ECs in the specific tissue can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in that specific tissue.

In specific embodiments:
(a) venuleness of an EC may be increased in peripheral lymph node by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in peripheral lymph node; such a gene may be selected from one or more of the genes listed in Table 22 and/or Table 56; or
(b) venuleness of an EC may be increased in mesenteric lymph node by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in mesenteric lymph node; such a gene may be selected from one or more of the genes listed in Table 23 and/or Table 57; or
(c) venuleness of an EC may be increased in Peyer's patch by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in Peyer's patch; such a gene may be selected from one or more of the genes listed in Table 24 and/or Table 58; or
(d) venuleness of an EC may be increased in thymus by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in thymus; such a gene may be selected from one or more of the genes listed in Table 25 and/or Table 59; or
(e) venuleness of an EC may be increased in visceral adipose tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in visceral adipose tissue; such a gene may be selected from one or more of the genes listed in Table 26 and/or Table 60; or
(f) venuleness of an EC may be increased in small intestine by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in small intestine; such a gene may be selected from one or more of the genes listed in Table 27 and/or Table 61; or
(g) venuleness of an EC may be increased in colon by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in colon; such a gene may be selected from one or more of the genes listed in Table 28 and/or Table 62; or
(h) venuleness of an EC may be increased in subcutaneous adipose tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in subcutaneous adipose tissue; such a gene may be selected from one or more of the genes listed in Table 29 and/or Table 63; or
(i) venuleness of an EC may be increased in bone tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in bone tissue; such a gene may be selected from one or more of the genes listed in Table 30 and/or Table 64.
(i) venuleness of an EC may be increased in uterine tissue by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in uterine tissue; such a gene may be selected from one or more of the genes listed in Table 31 and/or Table 65.
(i) venuleness of an EC may be increased in brain parenchyma by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in brain parenchyma; such a gene may be selected from one or more of the genes listed in Table 32 and/or Table 66.
(i) venuleness of an EC may be increased in choroid plexus by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in choroid plexus; such a gene may be selected from one or more of the genes listed in Table 33 and/or Table 67.
(i) venuleness of an EC may be increased in pia matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in pia matter; such a gene may be selected from one or more of the genes listed in Table 34 and/or Table 68.
(i) venuleness of an EC may be increased in dura matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in NV-ECs compared to V-ECs in dura matter; such a gene may be selected from one or more of the genes listed in Table 35.

### Increasing Venuleness of ECs in Multiple Tissues

Venuleness of ECs may be increased in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by one or more of the methods described herein. In some embodiments, venuleness of an EC may be increased in multiple tissues by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in those multiple tissues. The expression level of the at least one gene that has higher expression level in V-ECs compared to NV-ECs in multiple tissues can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level of the gene in the EC in those multiple tissues.

In specific embodiments, venuleness of an EC may be increased in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Such a gene may be selected from one or more of the genes listed in Table 1, Table 2, Table 3, Table 36, Table 37, and/or Table 38. The expression level of the at least one gene that has higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or plasmid encoding an activating antibody, an activating antigen-binding fragment, an activating protein, and/or an activating peptide), or a viral vector (e.g., a lentivirus vector)) that increases the expression level of the gene in the EC in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. For example, the expression level of at least one cell surface molecule from Table 36, Table 37, and/or Table 38 that has higher expression level in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be increased by contacting the EC with an activating antibody directed against that cell surface molecule.

Alternatively, venuleness of an EC may be increased in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≤5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs in those multiple tissues. The expression level of the at least one gene that has higher expression level in NV-ECs compared to V-ECs in multiple tissues can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in those multiple tissues.

In specific embodiments, venuleness of an EC may be increased in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by reducing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Such a gene may be selected from one or more of the genes listed in Table 19, Table 20, Table 21, Table 53, Table 54, and/or Table 55. The expression level of the at least one gene that has higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be reduced by contacting the EC with one or more (e.g., 1, 2, 3, 4, 5, 6, 7, 8, 9, 10, or more) agent (e.g., a polypeptide (e.g., an inhibitory antibody or inhibitory antigen-binding fragment), a small molecule (e.g., a small molecule inhibitor), a nucleic acid molecule (e.g., an inhibitory RNA, such as an siRNA or shRNA), a nuclease (e.g., Cas9), a viral vector (e.g., a lentivirus vector), or a plasmid (e.g., a plasmid encoding an inhibitory antibody, an inhibitory antigen-binding fragment, an inhibitory protein, and/or an inhibitory peptide)) that reduces the expression level of the gene in the EC in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. For example, the expression level of at least one cell surface molecule from Table 37 and/or Table 38 that has higher expression level in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter can be reduced by contacting the EC with an inhibitory antibody directed against that cell surface molecule.

In different embodiments, venuleness of an EC may be increased in all of peripheral lymph node, mesenteric lymph node, and Peyer's patch by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥0.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in all of peripheral lymph node, mesenteric lymph node, and Peyer's patch, but not in other tissues (i.e., gene that has higher expression level in HEVs, such as HEV-specific gene). Such a gene may be selected from one or more of the genes listed in Table 17 and/or Tables 52. The expression level of the at least one gene that has higher expression level in V-ECs compared to NV-ECs in all of peripheral lymph node, mesenteric lymph node, and Peyer's patch, but not in other tissues (i.e., gene that has higher expression level in HEVs, such as HEV-specific gene) can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level of the gene in the EC in all of peripheral lymph node, mesenteric lymph node, and Peyer's patch. For example, the expression level of at least one cell surface molecule from Table 52 that has higher expression level in V-ECs compared to NV-ECs in all of peripheral lymph node, mesenteric lymph node, and Peyer's patch can be increased by contacting the EC with an activating antibody directed against that cell surface molecule.

In additional embodiments, venuleness of an EC may be increased in all of mesenteric lymph node, Peyer's patch, small intestine, and colon by increasing the expression level of at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has ≥1.9 (e.g., ≥2.0, ≥2.1, ≥2.2, ≥2.3, ≥2.4, ≥2 0.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher expression level in V-ECs compared to NV-ECs in all of mesenteric lymph node, Peyer's patch, small intestine, and colon, but not in other tissues (i.e., gene that has higher expression level in gut V-ECs, such as gut V-EC specific gene). Such a gene may be the gene listed in Table 18. The expression level of the at least one gene that has higher expression level in V-ECs compared to NV-ECs in all of mesenteric lymph node, Peyer's patch, small intestine, and colon, but not in other tissues (i.e., gene that has higher expression level in gut V-ECs, such as gut V-EC specific gene) can be increased by contacting the EC with one or more (e.g., 1, 2, 3, 4, 5, 6, 7, 8, 9, 10, or more) agent (e.g., a polypeptide (e.g., an activating antibody or activating antigen-binding fragment), a small molecule (e.g., a small molecule activator), a nucleic acid molecule (e.g., an activating RNA, such as an saRNA), a plasmid (e.g., an overexpression plasmid or a plasmid encoding an activating antibody, an activating antigen-binding fragment, an activating protein, and/or an activating peptide), or a viral vector (e.g., a lentivirus vector)) that increases the expression level of the gene in the EC in all of mesenteric lymph node, Peyer's patch, small intestine, and colon.

Increasing venuleness by one or more methods described herein (e.g., increase in expression level of V-EC-specific genes or decrease in expression level of NV-EC specific genes) may also induce venular differentiation (i.e., induce differentiation of ECs into V-ECs), leading to an increase in venular functions, venular phenotypes (e.g., HEV phenotype, such as increase in HEV phenotype by increasing the expression level of HEV-specific genes) and venule-specific functional properties of ECs. In specific embodiments, increase in expression level of HEV-specific genes, i.e., genes that have higher expression level in V-ECs compared to NV-ECs in all of peripheral lymph node, mesenteric lymph node, and Peyer's patch, but not in other tissues, may increase HEV phenotype. A plausible outcome of such increase in venuleness, venular differentiation and HEV phenotype is increased leukocyte recruitment, which may have a therapeutic benefit in cancer treatment by promoting anti-tumor immunity. Additionally, increase of venuleness and venular differentiation in specific tissues restricts its physiologic effect (e.g., increased inflammation) to that specific tissue only, thus reducing adverse effects associated with anti-cancer therapeutics that work in a non-targeted manner.

### III. Targeting ECs in Specific Tissues

Additionally, disclosed herein are genes that are differentially expressed in ECs (V-ECs and NV-ECs) in a specific tissue (e.g., one of peripheral lymph node, mesenteric lymph node, Peyer's patch, thymus, visceral adipose tissue, small intestine, colon, subcutaneous adipose tissue, or bone tissue). Also described are methods of targeting ECs in a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by targeting one or more of the genes that are differentially expressed in ECs in that specific tissue. These methods contemplate new therapeutic approaches for delivery of therapeutics (e.g., any of the agents described in the previous section, anti-inflammatory drug, or anti-cancer therapeutic) and/or diagnostics (e.g., a diagnostic dye) to a specific tissue by targeting ECs in that specific tissue, thus reducing the adverse effects that are associated with non-targeted delivery or systemic administration of therapeutics and/or diagnostics.

### Genes Differentially Expressed in ECs in Specific Tissues

Genes that are differentially expressed (e.g., have higher expression level) in ECs (V-ECs and NV-ECs) in a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) include:

(a) genes whose expression level in V-ECs and/or NV-ECs (e.g., in V-ECs or NV-ECs, or in V-ECs and NV-ECs combined) is ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher in a specific tissue compared to other tissues (e.g., one or more genes listed in Tables 69-82 (e.g., Table 69, Table 70, Table 71, Table 72, Table 73, Table 74, Table 75, Table 76, Table 77, Table 78, Table 79, Table 80, Table 81, and/or Table 82), and/or Tables 83-96 (e.g., Table 83, Table 84, Table 85, Table 86, Table 87, Table 88, Table 89, Table 90, Table 91, Table 92, Table 93, Table 94, Table 95, and/or Table 96)); and

(b) genes whose expression level in both V-ECs and NV-ECs is ≥1.9 (e.g., ≥2.0, ≥2.1, ≥2.2, ≥2.3, ≥2.4, ≥2.5, ≥2.6, ≥2.7, ≥2.8, ≥2.9, ≥3.0, ≥4.0, ≥5.0, ≥6.0, ≥7.0, ≥8.0, ≥9.0, ≥10.0, ≥15.0, ≥20.0, ≥25.0, ≥30.0, ≥35.0, ≥40.0, ≥45.0, ≥50.0, ≥55.0, ≥60.0, ≥65.0, ≥70.0, ≥75.0, ≥80.0, ≥85.0, ≥90.0, ≥95.0, ≥100.0, or more) fold higher in a specific tissue compared to other tissues and fold change comparison S 1.5 between V-ECs and NV-ECs in the given tissue (e.g., one or more genes listed in Tables 97-110 (e.g., Table 97, Table 98, Table 99, Table 100, Table 101, Table 102, Table 103, Table 104, Table 105, Table 106, Table 107, Table 108, Table 109, and/or Table 110), and/or Tables 111-121 (e.g., Table 111, Table 112, Table 113, Table 114, Table 115, Table 116, Table 117, Table 118, Table 119, Table 120, and/or Table 121)).

### Targeting ECs in a Specific Tissue

ECs in a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) may be targeted by targeting at least one (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, or more) gene that has higher expression level in ECs (V-ECs and NV-ECs) in that specific tissue. Such a gene may be selected from one or more of the genes listed in Tables 69-121 (e.g., one or more genes listed in Tables 69-82 (e.g., Table 69, Table 70, Table 71, Table 72, Table 73, Table 74, Table 75, Table 76, Table 77, Table 78, Table 79, Table 80, Table 81, and/or Table 82), Tables 83-96 (e.g., Table 83, Table 84, Table 85, Table 86, Table 87, Table 88, Table 89, Table 90, Table 91, Table 92, Table 93, Table 94, Table 95, and/or Table 96), Tables 97-110 (e.g., Table 97, Table 98, Table 99, Table 100, Table 101, Table 102, Table 103, Table 104, Table 105, Table 106, Table 107, Table 108, Table 109, and/or Table 110), and/or Tables 111-121 (e.g., Table 111, Table 112, Table 113, Table 114, Table 115, Table 116, Table 117, Table 118, Table 119, Table 120, and/or Table 121)). EC in a specific tissue may be targeted with an EC targeting molecule that targets (e.g., binds to) at least one gene that has higher expression level in ECs (V-ECs and NV-ECs) in that specific tissue compared to other tissues. In some embodiments, the EC targeting molecule may be a targeting antibody that binds to at least one cell surface molecule selected from those listed in Tables 83-96 or Tables 111-121 that has higher expression level in ECs in a specific tissue compared to the other tissues. For example, ECs in a specific tissue may be targeted with a targeting antibody that specifically binds to at least one cell surface molecule selected from those listed in Tables 83-96 or Tables 111-121 that has higher expression level in ECs in that specific tissue.

In specific embodiments,
(a) ECs in peripheral lymph node may be targeted with an EC targeting molecule that targets at least one gene that has higher expression level in ECs (V-ECs and NV-ECs) in peripheral lymph node; such a gene may be selected from one or more of the genes listed in Table 69, Table 83, Table 97, or Table 111; the EC targeting molecule may be a targeting antibody that binds to at least one cell surface molecule selected from those listed in Table 83 or Table 111;
(b) ECs in mesenteric lymph node may be targeted with an EC targeting molecule that targets at least one gene that has higher expression level in ECs (V-ECs and NV-ECs) in mesenteric lymph node; such a gene may be selected from one or more of the genes listed in Table 70, Table 84, or Table 98; the EC targeting molecule may be a targeting antibody that binds to at least one cell surface molecule selected from those listed in Table 84;
(c) ECs in Peyer's patch may be targeted with an EC targeting molecule that targets at least one gene that has higher expression level in ECs (V-ECs and NV-ECs) in Peyer's patch; such a gene may be selected from one or more of the genes listed in Table 71, Table 85, Table 99, or Table 112; the EC targeting molecule may be a targeting antibody that binds to at least one cell surface molecule selected from those listed in Table 85 or Table 112;
(d) ECs in thymus may be targeted with an EC targeting molecule that targets at least one gene that has higher expression level in ECs (V-ECs and NV-ECs) in thymus; such a gene may be selected from one or more of the genes listed in Table 72, Table 86, Table 100, or Table 113; the EC targeting molecule may be a targeting antibody that binds to at least one cell surface molecule selected from those listed in Table 86 or Table 113;
(e) ECs in visceral adipose tissue may be targeted with an EC targeting molecule that targets at least one gene that has higher expression level in ECs (V-ECs and NV-ECs) in visceral adipose tissue; such a gene may be selected from one or more of the genes listed in Table 73, Table 87, or Table 101; the EC targeting molecule may be a targeting antibody that binds to at least one cell surface molecule selected from those listed in Table 87;
(f) ECs in small intestine may be targeted with an EC targeting molecule that targets at least one gene that has higher expression level in ECs (V-ECs and NV-ECs) in small intestine; such a gene may be selected from one or more of the genes listed in Table 74, Table 88, Table 102, or Table 114; the EC targeting molecule may be a targeting antibody that binds to at least one cell surface molecule selected from those listed in Table 88 or Table 114;
(g) ECs in colon may be targeted with an EC targeting molecule that targets at least one gene that has higher expression level in ECs (V-ECs and NV-ECs) in colon; such a gene may be selected from one or more of the genes listed in Table 75, Table 89, Table 103, or Table 115; the EC targeting molecule may be a targeting antibody that binds to at least one cell surface molecule selected from those listed in Table 89 or Table 115;
(h) ECs in subcutaneous adipose tissue may be targeted with an EC targeting molecule that targets at least one gene that has higher expression level in ECs (V-ECs and NV-ECs) in subcutaneous adipose tissue; such a gene may be selected from one or more of the genes listed in Table 76, Table 90, Table 104, or Table 116; the EC targeting molecule may be a targeting antibody that binds to at least one cell surface molecule selected from those listed in Table 90 or Table 116;
(i) ECs in bone tissue may be targeted with an EC targeting molecule that targets at least one gene that has higher expression level in ECs (V-ECs and NV-ECs) in bone tissue; such a gene may be selected from one or more of the genes listed in Table 77, Table 91, Table 105, or Table 117; the EC targeting molecule may be a targeting antibody that binds to at least one cell surface molecule selected from those listed in Table 91 or Table 117.
(j) ECs in uterus may be targeted with an EC targeting molecule that targets at least one gene that has higher expression level in ECs (V-ECs and NV-ECs) in uterus; such a gene may be selected from one or more of the genes listed in Table 78, Table 92, Table 106, or Table 118; the EC targeting molecule may be a targeting antibody that binds to at least one cell surface molecule selected from those listed in Table 92 or Table 118.
(k) ECs in brain parenchyma may be targeted with an EC targeting molecule that targets at least one gene that has higher expression level in ECs (V-ECs and NV-ECs) in brain parenchyma; such a gene may be selected from one or more of the genes listed in Table 79, Table 93, Table 107, or Table 119; the EC targeting molecule may be a targeting antibody that binds to at least one cell surface molecule selected from those listed in Table 93 or Table 119.
(l) ECs in choroid plexus may be targeted with an EC targeting molecule that targets at least one gene that has higher expression level in ECs (V-ECs and NV-ECs) in choroid plexus; such a gene may be selected from one or more of the genes listed in Table 80, Table 94, Table 108, or Table 120; the EC targeting molecule may be a targeting antibody that binds to at least one cell surface molecule selected from those listed in Table 94 or Table 120.
(m) ECs in pia matter may be targeted with an EC targeting molecule that targets at least one gene that has higher expression level in ECs (V-ECs and NV-ECs) in pia matter; such a gene may be selected from one or more of the genes listed in Table 81, Table 95, or Table 109; the EC targeting molecule may be a targeting antibody that binds to at least one cell surface molecule selected from those listed in Table 95.
(n) ECs in dura matter may be targeted with an EC targeting molecule that targets at least one gene that has higher expression level in ECs (V-ECs and NV-ECs) in dura matter; such a gene may be selected from one or more of the genes listed in Table 82, Table 96, Table 110, or Table 121; the EC targeting molecule may be a targeting antibody that binds to at least one cell surface molecule selected from those listed in Table 96 or Table 121.

Various therapeutics (e.g., any of the agents described in the previous section, anti-inflammatory drug, or cancer therapeutic) and/or diagnostics (e.g., diagnostic dyes) may be delivered to a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by one or more of these EC targeting methods. In particular, such therapeutics and/or diagnostics may be targeted to (e.g., delivered to) a specific tissue by conjugating (e.g., bioconjugating) the therapeutics and/or diagnostics with one or more EC targeting molecules that targets ECs of that specific tissue.

### IV. Detecting Venuleness

Additionally, disclosed herein are methods for detecting venuleness. Venuleness of ECs may be detected by detecting the expression of one or more specific markers of venuleness (e.g., DARC). Venuleness of a cell (e.g., an EC) may be confirmed by the presence of one or more of the specific markers of venuleness. Alternatively, absence of venuleness (i.e., non-venuleness) of a cell (e.g., an EC) may be confirmed by absence of one or more of the specific markers of venuleness. Presence or absence of one or more specific markers of venuleness can be detected by using a detecting molecule. In some embodiments, the detecting molecule used to detect the presence or absence of one or more specific markers of venuleness is a detecting antibody. Such an antibody may be directed against a specific protein marker of venuleness, such as an antibody directed against DARC. In particular embodiments, the detecting antibody may detect expression of one or more specific markers of venuleness by using one or more technical platforms (e.g., flow cytometry, western blot, immunohistochemistry, or ELISA). For example, detecting antibody directed against DARC may detect the expression of DARC by both flow cytometry and immunohistochemistry. In alternative embodiments, RNA-sequencing, DNA-sequencing, or other nucleic acid hybridization-based methods known in the art may be used for detection of specific markers of venuleness (e.g., mRNA markers of venuleness). One or more of such methods may be used alone, or in combination to identify, distinguish, detect, or sort out cells exhibiting venuleness (e.g., V-ECs).

Alternatively, venuleness of an EC may also be determined by determining the expression level of one or more genes that are indicative of venuleness, such as one or more genes that are selectively expressed in V-ECs or NV-ECs. For example, high expression level of one or more genes that have higher expression level in V-ECs compared to NV-ECs, or low expression level of one or more genes that have higher expression level in NV-ECs compared to V-ECs may indicate venuleness of an EC. Expression of genes that are indicative of venuleness may be determined by transcriptome analysis, RNA-sequencing, DNA-sequencing, or other nucleic acid hybridization-based methods known in the art.

### V. Modulating Inflammation

Featured herein are methods for modulating (e.g., reducing or increasing) inflammation by modulating (e.g., reducing or increasing) the venuleness of ECs according to one or more of the methods described in the foregoing sections.

### Reducing Inflammation

Described herein are methods of reducing inflammation by reducing venuleness of ECs following one or more of the methods described in the foregoing sections.

In some embodiments, inflammation may be reduced in a tissue-specific manner (e.g., in a specific tissue) by one or more of the methods described herein. In some embodiments, inflammation may be reduced in a specific tissue (e.g., peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by reducing the venuleness of ECs in that specific tissue. In specific embodiments:
(a) inflammation may be reduced in peripheral lymph node by reducing the venuleness of ECs in peripheral lymph node following one or more of the methods described in the foregoing sections;
(b) inflammation may be reduced in mesenteric lymph node by reducing the venuleness of ECs in mesenteric lymph node following one or more of the methods described in the foregoing sections;
(c) inflammation may be reduced in Peyer's patch by reducing the venuleness of ECs in Peyer's patch following one or more of the methods described in the foregoing sections;
(d) inflammation may be reduced in thymus by reducing the venuleness of ECs in thymus following one or more of the methods described in the foregoing sections;
(e) inflammation may be reduced in visceral adipose tissue by reducing the venuleness of ECs in visceral adipose tissue following one or more of the methods described in the foregoing sections;
(f) inflammation may be reduced in small intestine by reducing the venuleness of ECs in small intestine following one or more of the methods described in the foregoing sections;
(g) inflammation may be reduced in colon by reducing the venuleness of ECs in colon following one or more of the methods described in the foregoing sections;
(h) inflammation may be reduced in subcutaneous adipose tissue by reducing the venuleness of ECs in subcutaneous adipose tissue following one or more of the methods described in the foregoing sections;
(i) inflammation may be reduced in bone tissue by reducing the venuleness of ECs in bone tissue following one or more of the methods described in the foregoing sections;
(j) inflammation may be reduced in uterus by reducing the venuleness of ECs in uterus following one or more of the methods described in the foregoing sections;
(k) inflammation may be reduced in brain parenchyma by reducing the venuleness of ECs in brain parenchyma following one or more of the methods described in the foregoing sections;
(l) inflammation may be reduced in choroid plexus by reducing the venuleness of ECs in choroid plexus following one or more of the methods described in the foregoing sections;
(m) inflammation may be reduced in pia matter by reducing the venuleness of ECs in pia matter following one or more of the methods described in the foregoing sections;
(n) inflammation may be reduced in dura matter by reducing the venuleness of ECs in dura matter following one or more of the methods described in the foregoing sections.

In different embodiments, inflammation may be reduced in multiple (e.g., in more than one, such as in 2, 3, 4, 5, 6, 7, 8, or in all 9 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by one or more of the methods described herein. In particular, inflammation may be reduced in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by reducing venuleness of ECs in those multiple tissues following one or more of the methods described in the previous sections. In specific embodiments, inflammation may be reduced in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by reducing venuleness of ECs in in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter following one or more of the methods described in the previous sections.

In other embodiments, inflammation may be reduced by reducing HEV phenotype of ECs following one or more of the methods described in the previous sections. In specific embodiments, inflammation may be reduced in peripheral lymph node, mesenteric lymph node, and Peyer's patch by reducing HEV phenotype of ECs in peripheral lymph node, mesenteric lymph node, and Peyer's patch following one or more of the methods described in the previous sections. Inflammation may be reduced in cells (e.g., in ECs) in a subject (e.g., a human, such as a human with one or more inflammatory diseases) either globally (i.e., in multiple tissues) or in a tissue-specific manner by one or more of the methods described herein. Uncontrolled inflammation is the underlying cause of many inflammatory diseases, including, but not restricted to endotoxemia, sepsis, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischaemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Retier's syndrome, and Hodgkin's disease. Reducing inflammation either globally, or in a tissue-specific manner, by one or more of the methods described herein may thus provide therapeutic approach for treatment of a subject (e.g., a human) with one or more of such inflammatory diseases.

Inflammation is a complex biological response to a variety of noxious stimuli. It is absolutely critical to the pathogenesis of inflammation that V-ECs possess the ability to support tissue-specific multi-step adhesion cascades to recruit blood-borne leukocytes to the extravascular compartment. Indeed, leukocyte recruitment is thought to be essential for autoimmune and inflammatory diseases that can target virtually any tissue, such as psoriasis in the skin, inflammatory bowel diseases in the small intestine and colon, multiple sclerosis in the brain and spinal cord, various forms of arthritis in joints and synovium and juvenile diabetes in the pancreas, to name a few. Understanding the molecular mechanisms regulating leukocyte recruitment in health and disease may provide opportunities for the development of novel treatments for inflammation-related diseases. Current anti-inflammatory drugs, such as corticoids, non-steroidal drugs and biologics like anti-TNF or anti-alpha 4 integrin antibodies act systemically and thus affect healthy and damaged tissues alike. Adverse side effects comprise gastrointestinal and renal effects as well as in some cases, an increased susceptibility to infection linked to impaired leukocyte trafficking in healthy tissue. There is no FDA-approved anti-inflammatory drug that targets selectively the endothelium, not to mention tissue-specific vascular beds that are promoting inflammation. Hence there is unmet need in the field to develop therapeutic approaches that would minimize such adverse effects by restricting the decrease of inflammation to targeted tissue. The methods of reducing inflammation in a tissue-specific manner described herein may serve to that need.

### Increasing Leukocyte Recruitment

Described herein are methods of increasing leukocyte recruitment by increasing venuleness of ECs following one or more of the methods described in the foregoing sections.

In some embodiments, leukocyte recruitment may be increased in a tissue-specific manner (e.g., in a specific tissue) by one or more of the methods described herein. In some embodiments, leukocyte recruitment may be increased in a specific tissue (e.g., peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by increasing the venuleness of ECs in that specific tissue. In specific embodiments:
(a) leukocyte recruitment may be increased in peripheral lymph node by increasing the venuleness of ECs in peripheral lymph node following one or more of the methods described in the foregoing sections;
(b) leukocyte recruitment may be increased in mesenteric lymph node by increasing the venuleness of ECs in mesenteric lymph node following one or more of the methods described in the foregoing sections;
(c) leukocyte recruitment may be increased in Peyer's patch by increasing the venuleness of ECs in Peyer's patch following one or more of the methods described in the foregoing sections; (d) leukocyte recruitment may be increased in thymus by increasing the venuleness of ECs in thymus following one or more of the methods described in the foregoing sections;
(e) leukocyte recruitment may be increased in visceral adipose tissue by increasing the venuleness of ECs in visceral adipose tissue following one or more of the methods described in the foregoing sections;
(f) leukocyte recruitment may be increased in small intestine by increasing the venuleness of ECs in small intestine following one or more of the methods described in the foregoing sections;
(g) leukocyte recruitment may be increased in colon by increasing the venuleness of ECs in colon following one or more of the methods described in the foregoing sections;
(h) leukocyte recruitment may be increased in subcutaneous adipose tissue by increasing the venuleness of ECs in subcutaneous adipose tissue following one or more of the methods described in the foregoing sections;
(i) leukocyte recruitment may be increased in bone tissue by increasing the venuleness of ECs in bone tissue following one or more of the methods described in the foregoing sections;
(j) leukocyte recruitment may be increased in uterus by increasing the venuleness of ECs in uterus following one or more of the methods described in the foregoing sections;
(k) leukocyte recruitment may be increased in brain parenchyma by increasing the venuleness of ECs in brain parenchyma following one or more of the methods described in the foregoing sections;
(l) leukocyte recruitment may be increased in choroid plexus by increasing the venuleness of ECs in choroid plexus following one or more of the methods described in the foregoing sections;
(m) leukocyte recruitment may be increased in pia matter by increasing the venuleness of ECs in pia matter following one or more of the methods described in the foregoing sections;
(n) leukocyte recruitment may be increased in dura matter by increasing the venuleness of ECs in dura matter following one or more of the methods described in the foregoing sections.

In different embodiments, leukocyte recruitment may be increased in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) tissues by one or more of the methods described herein. In particular, leukocyte recruitment may be increased in multiple (e.g., in more than one, such as in 2, 3, 4, 5, 6, 7, 8, 9, 10, 11, 12, 13, or in all 14 of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) tissues by increasing venuleness of ECs in those multiple tissues following one or more of the methods described in the previous sections. In specific embodiments, leukocyte recruitment may be increased in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter by increasing venuleness of ECs in in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter following one or more of the methods described in the previous sections.

In other embodiments, leukocyte recruitment may be increased by increasing HEV phenotype of ECs following one or more of the methods described in the previous sections. In specific embodiments, leukocyte recruitment may be increased in peripheral lymph node, mesenteric lymph node, and Peyer's patch by increasing HEV phenotype of ECs in peripheral lymph node, mesenteric lymph node, and Peyer's patch following one or more of the methods described in the previous sections.

Leukocyte recruitment may be increased in cells (e.g., in ECs) in a subject (e.g., a human, such as a human with one or more cancers) either globally (i.e., in multiple tissues) or in a tissue-specific manner by one or more of the methods described herein. Leukocyte recruitment plays a major role in elimination of tumor cells. Many cancers (e.g., multiple myeloma, breast cancer, acute myelogenous leukemia (AML), acute lymphoblastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, prostate cancer, kidney cancer, renal cell carcinoma (RCC), esophageal cancer, melanoma, glioma, pancreatic cancer, ovarian cancer, gastrointestinal stromal tumors (GIST), sarcoma, estrogen receptor-positive (ERpos) breast cancer, lung cancer, non-small cell lung carcinoma (NSCLC), mesothelioma, intestinal cancer, colon cancer, bladder cancer, adrenal cancer, gallbladder cancer, or squamous cell carcinoma of the head and neck (SCCHN)) reduce leukocyte recruitment in the tumor microenvironment in order to evade the host immune system. Boosting leukocyte recruitment and anti-tumor immunity is thus the goal of many onco-immunotherapy drugs. Increasing leukocyte recruitment either globally, or in a tissue-specific manner, by one or more of the methods described herein may thus provide therapeutic approach for treatment of a subject (e.g., a human) with one or more cancers.

Recently, the American Cancer Society revealed that cancer has become the number one cause of death in 21 states and projected 1.7 million new cancer cases will arise in the U.S. for 2016 and 8.2 million cancer related deaths worldwide. Advances in immuno-oncology indicate that a cancer patient's immune system can be therapeutically harnessed to eliminate malignant tumors, providing long lasting responses in some cancers. However, despite this progress, current immunotherapy regimens have only shown efficacy in a subset of malignancies and/or a minority of patients. The high failure rate of cancer immunotherapy is inversely correlated with the presence of tumor-infiltrating T cells. The reason(s) for the paucity of T cells in so-called non-inflammatory tumors (which have a poor prognosis) are not well understood, but likely involve defects in T cell recruitment, i.e., the inability of circulating tumor antigen-specific T cells to adhere to and emigrate from tumor microvessels into the surrounding tissue. The only current treatments targeting tumor vasculature aim to inhibit angiogenesis by targeting VEGF, but this approach does not promote venular differentiation. There are no FDA-approved anti-tumoral drugs that can selectively boost EC dependent immune cell recruitment. Since V-ECs are the principal gatekeepers for leukocyte emigration, drugs that promote venuleness of ECs or V-EC differentiation could potentially boost tumor infiltration by T cells and thus enhance onco-immunotherapy. Therefore, the neovasculature of solid tumors may be inherently suboptimal at recruiting T cells because of inadequate endothelial differentiation into functional venular type microvessels. Therapeutic approaches aimed at inducing a venular programing in ECs that would promote venule formation and/or venuleness and consequently boost anti-tumor immunity is thus the unmet need of the field. The methods of increasing leukocyte recruitment by one or more of the methods described herein may serve to that need.

### VI. Treatment of Diseases

Also featured here are methods of treating diseases (e.g., inflammatory diseases or cancers) by modulating (e.g., reducing or increasing) inflammation by means of one or more of the methods described herein.

Inflammation is part of the complex biological response of body tissues to harmful stimuli, such as pathogens, damaged cells, cancer/tumor cells, stress, or irritants, and is a protective response involving immune cells, blood vessels, and molecular mediators. The function of inflammation is to eliminate the initial cause of cell injury (e.g., pathogens, or cancer cells), clear out necrotic cells and tissues damaged from the original insult and the inflammatory process, and initiate tissue repair. However, inflammation acts as a double-edged sword. Uncontrolled inflammation, inflammation that persists too long, and/or is self-directed, can cause severe tissue damages, leading to deepening, broadening and worsening of tissue injury, irreversible tissue necrosis and complete loss of functionality. Inflammation thus regulate the pathogenesis of many diseases. Inflammation represents the consequence of capillary dilation with accumulation of fluid (edema) and the recruitment of leukocytes. While reduced leukocyte recruitment and inflammation is the goal of some therapeutic approaches (e.g., in inflammatory diseases), increased leukocyte recruitment is the desired outcome in others (e.g., boosting anti-tumor immunity in cancer therapy).

Leukocyte migration through activated venular walls, also known as leukocyte recruitment, is a fundamental immune response that is prerequisite to the entry of effector cells such as neutrophils, monocytes, and effector T cells to sites of infection, injury, and stress within the interstitium. During the recruitment or trafficking process, leukocytes adhere to the vascular endothelium, and subsequently leave the circulation by transendothelial migration driven by chemoattractants, a process known as diapedesis. Reversible adherence of leukocytes to endothelium, basement membranes, and other surfaces on which they crawl is an essential event in the establishment of inflammation. The primary step in leukocyte recruitment is the establishment of weak and transient adhesive interactions between leukocytes and ECs of postcapillary venular walls in close vicinity to inflamed tissues. This facilitates in situ stimulation of leukocytes by EC-presented chemoattractants displayed on the luminal side of blood vessels, propagating firm leukocyte arrest, adhesion strengthening, crawling, and subsequently migration of cells out of the blood vasculature. This series of sequential but overlapping steps termed the leukocyte-adhesion cascade, is primarily mediated by two major adhesion receptor families, selectins (expressed on leukocytes and ECs) and integrins (expressed on leukocytes). Activation of ECs, and differentiation of ECs to V-ECs that support leukocyte recruitment is a decisive step in this process. Hence, modulation (e.g., decrease or increase) of venuleness, venular differentiation, and HEV phenotype by one or more of the methods described herein can eventually lead to decreased or increased leukocyte recruitment, resulting in decreased or increased inflammation, which may eventually have therapeutic benefits in treatment of inflammatory diseases or cancers.

### Treatment of Inflammatory Diseases

In specific embodiments, reduction or decrease of venuleness, venular differentiation and HEV phenotype by one or more of the methods described herein can eventually lead to decreased leukocyte recruitment, resulting in decreased inflammation, which may eventually have therapeutic benefits in treatment of inflammatory diseases. Inflammatory diseases that can be treated by these methods include, but are not restricted to one or more of endotoxemia, sepsis, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischaemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Retier's syndrome, or Hodgkin's disease.

### Treatment

In some embodiments, one or more of the inflammatory diseases may be treated by administering to a subject (e.g., a human, such as a human with one or more inflammatory diseases) an effective amount of an agent that reduces venuleness (e.g., an agent that decreases the expression level of one or more of the genes having higher expression level in V-ECs compared to NV-ECs, or an agent that increases the expression level of one or more of the genes having higher expression level in NV-ECs compared to V-ECs) or an agent that decreases HEV phenotype (e.g., an agent that decreases the expression level of one or more of the HEV-specific genes, i.e., genes having higher expression level in V-ECs in peripheral lymph node, mesenteric lymph node, and Peyer's patch), and thereby reduces inflammation. In some embodiments, the agent may be conjugated to one or more of the EC targeting molecules (e.g., EC targeting antibodies) described herein to target the agent to EC in a specific tissue. In some embodiments, the agent or the agent/EC targeting molecule conjugate may be administered to a subject in combination with one or more anti-inflammatory drugs (e.g., one or more of a disease-modifying anti-rheumatic drug (DMARD), a biologic response modifier (a type of DMARD), a corticosteroid, a nonsteroidal anti-inflammatory medication (NSAID), prednisone, prednisolone, methylprednisolone, methotrexate, hydroxychloroquine, sulfasalazine, leflunomide, cyclophosphamide, azathioprine, tofacitinib, adalimumab, abatacept, anakinra, kineret, certolizumab, etanercept, golimumab, infliximab, rituximab tocilizumab, an antiviral compound, a nucleoside-analog reverse transcriptase inhibitor (NRTI), a non-nucleoside reverse transcriptase inhibitor (NNRTI), an antibacterial compound, an antifungal compound, or an antiparasitic compound).

### Effects of Treatment

In some embodiments, administration of the agent that decreases venuleness (e.g., an agent that decreases the expression level of one or more of the genes having higher expression level in V-ECs compared to NV-ECs, or an agent that increases the expression level of one or more of the genes having higher expression level in NV-ECs compared to V-ECs) or the agent that decreases HEV phenotype (e.g., an agent that decreases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-inflammatory drug(s) to a subject (e.g., a human) or a cell (e.g., an immune cell) may reduce leukocyte recruitment and/or various immune cell activities.

Exaggerated leukocyte recruitment or trafficking into specific peripheral tissues is associated with a multitude of pathologies. For example, excessive neutrophil accumulation in peripheral tissues contributes to the development of ischemia-reperfusion injury, such as that observed during acute myocardial infarction, stroke, shock, acute respiratory distress syndrome and many inflammatory diseases. Excessive Th1 inflammation characterized by tissue infiltration of interferon-gamma secreting effector T cells and activated macrophages is associated with atherosclerosis, allograft rejection, hepatitis, and multiple autoimmune diseases including multiple sclerosis, rheumatoid arthritis, psoriasis, Crohn's disease, type 1 diabetes and lupus erythematodes. Excessive Th2 inflammation characterized by tissue infiltration of IL-4, IL-5, and IL-13 secreting Th2 cells, eosinophils and mast cells is associated with asthma, food allergies and atopic dermatitis.

In some embodiments, administration of the agent that decreases venuleness (e.g., an agent that decreases the expression level of one or more of the genes having higher expression level in V-ECs compared to NV-ECs, or an agent that increases the expression level of one or more of the genes having higher expression level in NV-ECs compared to V-ECs) or the agent that decreases HEV phenotype (e.g., an agent that decreases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-inflammatory drug(s) to a subject (e.g., a human) or a cell (e.g., an immune cell) may reduce leukocyte trafficking or recruitment. Leukocyte recruitment can be assessed by measuring the number of leukocytes in a location of interest (e.g., a lymph node or secondary lymphoid organ, or site of inflammation). Leukocyte recruitment can also be assessed by measuring a chemokine, receptor, or marker associated with leukocyte recruitment known in the art (e.g., integrins, immunoglobulin cell-adhesion molecules (IgSF CAMs), cadherins, selectins, and a family of small cytokines called chemokines). In certain embodiments, the parameter is decreased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is decreased in the subject between 5-20%, between 5-50%, between 10-50%, between 20-80%, between 20-70%, between 50-200%, between 100%-500%.

In some embodiments, administration of the agent that decreases venuleness (e.g., an agent that decreases the expression level of one or more of the genes having higher expression level in V-ECs compared to NV-ECs, or an agent that increases the expression level of one or more of the genes having higher expression level in NV-ECs compared to V-ECs) or the agent that decreases HEV phenotype (e.g., an agent that decreases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-inflammatory drug(s) to a subject (e.g., a human) or a cell (e.g., an immune cell) may reduce one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; monocyte proliferation; macrophage proliferation; dendritic cell proliferation; NK cell proliferation; ILC proliferation, mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation, ILC ADCC, ILC degranulation, cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of HEVs; or development of ectopic or tertiary lymphoid organs (TLOs). In certain embodiments, the immune response (e.g., an immune cell activity listed herein) is decreased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the immune response is decreased in the subject or cell between 5-20%, between 5-50%, between 10-50%, between 20-80%, between 20-70%, between 50-200%, between 100%-500%.

### Treatment of Cancers

In alternative embodiments, increase of venuleness, venular differentiation and HEV phenotype by one or more of the methods described herein can eventually lead to increased leukocyte recruitment, resulting in increased inflammation, which may eventually have therapeutic benefits in treatment of cancers.

### Cancer Types

Cancers that can be treated by these methods may be any solid or liquid cancer and includes benign or malignant tumors, and hyperplasias, including gastrointestinal cancer (such as non-metastatic or metastatic colorectal cancer, pancreatic cancer, gastric cancer, esophageal cancer, hepatocellular cancer, cholangiocellular cancer, oral cancer, lip cancer); urogenital cancer (such as hormone sensitive or hormone refractory prostate cancer, renal cell cancer, bladder cancer, penile cancer); gynecological cancer (such as ovarian cancer, cervical cancer, endometrial cancer); lung cancer (such as small-cell lung cancer and non-small-cell lung cancer); head and neck cancer (e.g., head and neck squamous cell cancer); CNS cancer including malignant glioma, astrocytomas, retinoblastomas and brain metastases; malignant mesothelioma; non-metastatic or metastatic breast cancer (e.g., hormone refractory metastatic breast cancer); skin cancer (such as malignant melanoma, basal and squamous cell skin cancers, Merkel Cell Carcinoma, lymphoma of the skin, Kaposi Sarcoma); thyroid cancer; bone and soft tissue sarcoma; and hematologic neoplasias (such as multiple myeloma, acute myelogenous leukemia, chronic myelogenous leukemia, myelodysplastic syndrome, acute lymphoblastic leukemia, Hodgkin's lymphoma).

Additional cancers that can be treated according to the methods described herein include breast cancer, lung cancer, stomach cancer, colon cancer, liver cancer, renal cancer, colorectal cancer, prostate cancer, pancreatic cancer, cervical cancer, anal cancer, vulvar cancer, penile cancer, vaginal cancer, testicular cancer, pelvic cancer, thyroid cancer, uterine cancer, rectal cancer, brain cancer, head and neck cancer, esophageal cancer, bronchus cancer, gallbladder cancer, ovarian cancer, bladder cancer, oral cancer, oropharyngeal cancer, larynx cancer, biliary tract cancer, skin cancer, a cancer of the central nervous system, a cancer of the respiratory system, and a cancer of the urinary system. Examples of breast cancers include, but are not limited to, triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Other cancers that can be treated according to the methods described herein include leukemia (e.g., B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), and erythroleukemia), sarcoma (e.g., angiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leiomyosarcoma, and neurofibrosarcoma), carcinoma (e.g., basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, squamous cell carcinoma, adenosquamous carcinoma, anaplastic carcinoma, adrenocortical carcinoma, cholangiocarcinoma, Merkel cell carcinoma, DCIS, and invasive ductal carcinoma), blastoma (e.g., hepatoblastoma, medulloblastoma, nephroblastoma, neuroblastoma, pancreatoblastoma, pleuropulmonary blastoma, retinoblastoma, and glioblastoma multiforme), lymphoma (e.g., Hodgkin's lymphoma, non-Hodgkin's lymphoma, and Burkitt lymphoma), myeloma (e.g., multiple myeloma, plasmacytoma, localized myeloma, and extramedullary myeloma), melanoma (e.g., superficial spreading melanoma, nodular melanoma, lentigo maligna melanoma, acral lentiginous melanoma, and amelanotic melanoma), neuroma (e.g., ganglioneuroma, Pacinian neuroma, and acoustic neuroma), glioma (e.g., astrocytoma, oligoastrocytoma, ependymoma, brainstem glioma, optic nerve glioma, and oligoastrocytoma), pheochromocytoma, meningioma, malignant mesothelioma, and virally induced cancer.

### Treatment

In some embodiments, one or more of the cancers may be treated by administering to a subject (e.g., a human, such as a human with one or more cancers) an effective amount of an agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-EVs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), and thereby increases leukocyte recruitment. In some embodiments, the agent may be conjugated to one or more of the EC targeting molecules (e.g., EC targeting antibodies) described herein to target the agent to ECs in a specific tissue. In some embodiments, the agent and/or the agent/EC targeting molecule conjugate may be administered to a subject in combination with one or more anti-cancer therapeutics (e.g., chemotherapeutics, targeted therapeutics, or immunotherapeutics).

### (i) Chemotherapeutics

One type of anti-cancer therapeutic that can be administered in combination with the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), and/or the agent/EC targeting molecule conjugate is a chemotherapeutic (e.g., a cytotoxic agent or other chemical compound useful in the treatment of cancer). These include anthracyclines (e.g., doxorubicin), nucleoside analogs (e.g., 5-fluorouracil (5-FU)) and related inhibitors, platinum-based anti-neoplastic agents (e.g., cisplatin), taxanes (e.g., paclitaxel), vinca alkaloids (e.g., vincristine), glycopeptide antibiotics (e.g., bleomycin), polypeptide antibiotic (e.g., actinomycin D), alkylating agents, antimetabolites, folic acid analogs, epipodophyllotoxins, L-asparaginase, topoisomerase inhibitors, interferons, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. Also included is leucovorin (LV), irenotecan, oxaliplatin, capecitabine, and docetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-FU; folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel; chloranbucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the first therapeutic agent described herein. Suitable dosing regimens of combination chemotherapies are known in the art.

### (ii) Targeted Anti-Cancer Therapeutics

Another type of anti-cancer therapeutic that can be administered in combination with the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), and/or the agent/EC targeting molecule conjugate is a targeted anti-cancer therapeutic. A targeted anti-cancer therapeutic blocks the growth of cancer cells by interfering with specific targeted molecules needed for carcinogenesis and tumor growth, rather than by simply interfering with all rapidly dividing cells (e.g., with traditional chemotherapeutic agents). Because most of these targeted therapeutics are biopharmaceuticals, the term biologic therapy is sometimes synonymous with targeted therapy when used in the context of cancer therapy (and thus distinguished from chemotherapy, that is, cytotoxic therapy). However, the modalities may be combined to enhance efficacy of the therapy. Targeted anti-cancer therapeutics include tyrosine kinase inhibitors, PI3K inhibitors, multi-kinase inhibitors, CDK4/6 inhibitors, mTOR inhibitors, NOTCH inhibitors, HSP90 inhibitors, HSP70 inhibitors, proteasome inhibitors, tumor metabolism inhibitors, Janus kinase inhibitors, ALK inhibitors, Bcl-2 inhibitors, VEGFR inhibitors, VEGF inhibitors, and serine/threonine kinase inhibitors among others.

(iii) Anti-Cancer Immunotherapeutics

Another type of anti-cancer therapeutic that can be administered in combination with the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), and/or the agent/EC targeting molecule conjugate is an anti-cancer immunotherapeutic. Anti-cancer immunotherapeutic are therapeutics (e.g., drugs, antibodies, cytokines) that modulate the immune system to treat cancer. The different kinds of anti-cancer immunotherapeutics that are currently used in cancer therapy include:

Cytokines: molecular messengers of the immune system that boost anti-tumor immunity by stimulating immune effector cells and stromal cells at the tumor site and enhancing tumor cell recognition by cytotoxic effector cells. Cytokines that are used to treat patients with cancers include interferons (IFNs, such as IFN-alpha) and interleukins (ILs, such as IL-2).

Monoclonal antibodies: man-made versions of immune system proteins that can be very useful in treating cancer as they are designed to attack cancer cells specifically.

Immune checkpoint inhibitors: drugs that take the ‘brakes’ off the immune system, thus helping it to recognize and attack cancer cells (e.g., PD-1 inhibitor, PD-L1 inhibitor, CTLA-4 inhibitor).

Inhibitor of the CBM (Carma1-BCL10-MALT-1) complex: Inhibitors that target the protease activity of MALT1 are useful in treating specific lymphoid malignancies.

Cancer vaccines: vaccines that either treat existing cancer or prevent development of a cancer.

Tumor-infiltrating lymphocytes (TILs): T cells that are naturally found in a patient's tumor (called tumor-infiltrating lymphocytes, TILs) are modified, enabling them to attack tumors more efficiently. TILs that best recognize the patient's tumor cells in laboratory tests are selected, expanded in number, activated by treatment with cytokines and other immune system signaling proteins and infused back into the patient's bloodstream.

CAR T-cell: T cells from a patient that are genetically modified in the laboratory to express a protein known as a chimeric antigen receptor, or CAR, before they are grown and infused into the patient. CARs are modified forms of T-cell receptor, which is expressed on the surface of T cells. The CARs are designed to allow the T cells to attach to specific proteins on the surface of the patient's cancer cells, thus improving their ability to attack the cancer cells.

Other, non-specific immunotherapies: treatments that boost the immune system in a general way, helping the immune system to attack cancer cells.

### Effects of Treatment

In some embodiments, administration of the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-cancer therapeutic(s) may increase leukocyte recruitment and/or various immune cell activities.

Insufficient leukocyte recruitment or trafficking into cancer tissues is the underlying cause for failure of many cancer therapies. The high failure rate of cancer immunotherapy is inversely correlated with the presence of tumor-infiltrating T cells. The reason(s) for the paucity of T cells in so-called non-inflammatory tumors (which have a poor prognosis) are not well understood, but likely involve the inability of circulating tumor antigen-specific T cells to adhere to and emigrate from tumor microvessels into the surrounding tissue.

In some embodiments, administration of the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-cancer therapeutic(s) to a subject (e.g., a human) or a cell (e.g., a cancer cell) may increase leukocyte trafficking or recruitment. Leukocyte recruitment can be assessed by measuring the number of leukocytes in a location of interest (e.g., a tumor). Leukocyte recruitment can also be assessed by measuring a chemokine, receptor, or marker associated with leukocyte recruitment known in the art (e.g., integrins, immunoglobulin cell-adhesion molecules (IgSF CAMs), cadherins, selectins, and a family of small cytokines called chemokines). In certain embodiments, the parameter is increased in the subject at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the parameter is increased in the subject between 5-20%, between 5-50%, between 10-50%, between 20-80%, between 20-70%, between 50-200%, between 100%-500%.

In some embodiments, administration of the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-cancer therapeutic(s) to a subject (e.g., a human) or a cell (e.g., a cancer cell) may increase one or more (e.g., 2 or more, 3 or more, 4 or more) of the following immune cell activities in the subject or cell: T cell polarization; T cell activation; dendritic cell activation; neutrophil activation; eosinophil activation; basophil activation; T cell proliferation; B cell proliferation; T cell proliferation; monocyte proliferation; macrophage proliferation; dendritic cell proliferation; NK cell proliferation; ILC proliferation, mast cell proliferation; neutrophil proliferation; eosinophil proliferation; basophil proliferation; cytotoxic T cell activation; circulating monocytes; peripheral blood hematopoietic stem cells; macrophage polarization; macrophage phagocytosis; macrophage ADCP, neutrophil phagocytosis; monocyte phagocytosis; mast cell phagocytosis; B cell phagocytosis; eosinophil phagocytosis; dendritic cell phagocytosis; macrophage activation; antigen presentation (e.g., dendritic cell, macrophage, and B cell antigen presentation); antigen presenting cell migration (e.g., dendritic cell, macrophage, and B cell migration); lymph node immune cell homing and cell egress (e.g., lymph node homing and egress of T cells, B cells, dendritic cells, or macrophages); NK cell activation; NK cell ADCC, mast cell degranulation; NK cell degranulation; ILC activation, ILC ADCC, ILC degranulation, cytotoxic T cell degranulation; neutrophil degranulation; eosinophil degranulation; basophil degranulation; neutrophil recruitment; eosinophil recruitment; NKT cell activation; B cell activation; regulatory T cell differentiation; dendritic cell maturation; development of HEVs; development of ectopic or tertiary lymphoid organs (TLOs); or lymph node or secondary lymphoid organ innervation. In certain embodiments, the immune response (e.g., an immune cell activity listed herein) is increased in the subject or cell at least 1%, 2%, 5%, 10%, 15%, 20%, 25%, 30%, 35%, 40%, 50%, 60%, 70%, 80%, 100%, 150%, 200%, 300%, 400%, 500% or more, compared to before the administration. In certain embodiments, the immune response is increased in the subject or cell between 5-20%, between 5-50%, between 10-50%, between 20-80%, between 20-70%, between 50-200%, between 100%-500%.

Administration of the agent that increases venuleness (e.g., an agent that increases the expression level of one or more of the genes having higher expression level in V-ECs compared to NV-ECs, or an agent that decreases the expression level of one or more of the genes having higher expression level in NV-ECs compared to V-ECs) or agent that increases HEV phenotype (e.g., an agent that increases the expression level of one or more of the HEV-specific genes, i.e., genes having higher expression level in V-ECs in peripheral lymph node, mesenteric lymph node, and Peyer's patch), the agent/EC targeting molecule conjugate, and/or the anti-cancer therapeutic(s) to a subject (e.g., a human) or a cell (e.g., a cancer cell) can also inhibit or decrease tumor growth, tumor cell proliferation, cancer cell growth, cancer cell proliferation, metastasis, migration, or invasion. Tumor growth, tumor cell proliferation, cancer cell growth, cancer cell proliferation, metastasis, migration, or invasion can be decreased in the subject or cancer cell culture at least 1%, 2%, 5%, 10%, 15%, 20%, 25%, 30%, 35%, 40%, 50%, 60%, 70%, 80%, 90%, 95%, 98%, or more, compared to before the administration. Tumor growth, tumor cell proliferation, cancer cell growth, cancer cell proliferation, metastasis, migration, or invasion can be decreased in the subject or cancer cell culture between 5-20%, between 5-50%, between 10-50%, between 20-70%, between 20-90%.

### Formulations and Carriers

In order to be administered to a subject, a pharmaceutical composition of the agent (e.g., an agent that reduces venuleness, agent reduces HEV phenotype, increases venuleness, or increases HEV phenotype) either alone or in combination with one or more EC targeting molecule can be formulated with a pharmaceutically acceptable carrier or excipient. A pharmaceutically acceptable carrier or excipient refers to a carrier (e.g., carrier, media, diluent, solvent, vehicle, etc.) which does not significantly interfere with the biological activity or effectiveness of the active ingredient(s) of a pharmaceutical composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, Remington: The Science and Practice of Pharmacy. 21st Edition. Philadelphia, PA. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For oral administration, agents can be formulated by combining the active compounds with pharmaceutically acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. For administration by inhalation, pharmaceutical compositions of this invention may be formulated in the form of an aerosol spray from a pressured container or dispenser, which contains a suitable propellant, e.g., a gas such as carbon dioxide, a fluorocarbon, or a nebulizer. Liquid or dry aerosol (e.g., dry powders, large porous particles, etc.) can also be used. For topical application, a pharmaceutical composition may be formulated in a suitable ointment, lotion, gel, or cream containing the active components suspended or dissolved in one or more pharmaceutically acceptable carriers suitable for use in such compositions.

Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015). Pharmaceutical compositions may be prepared in microcapsules, such as hydroxylmethylcellulose or gelatin-microcapsule and poly-(methylmethacrylate) microcapsule. Pharmaceutical compositions may also be prepared in other drug delivery systems such as liposomes, albumin microspheres, microemulsions, nanoparticles, and nanocapsules. Such techniques are described in Remington: The Science and Practice of Pharmacy 22^{th} edition (2012). The pharmaceutical compositions to be used for in vivo administration must be sterile. This is readily accomplished by filtration through sterile filtration membranes.

### Dosage and Routes of Administration

An agent (e.g., agent that reduces venuleness, agent that reduces HEV phenotype, agent that increases venuleness, or agent that increases HEV phenotype) either alone or in combination with one or more EC targeting molecule can be administered as a pharmaceutical composition to a subject (e.g., a human, such as a human with inflammatory disease or cancer) in a variety of ways. The composition must be suitable for the subject receiving the treatment, and the mode of administration. The composition used in this invention can be administered orally, sublingually, parenterally, intravenously, subcutaneously, intramedullary, intranasally, as a suppository, using a flash formulation, topically, intradermally, subcutaneously, via pulmonary delivery, via intra-arterial injection, or via a mucosal route. In specific embodiments, an agent (e.g., agent that reduces venuleness, agent that reduces HEV phenotype, or agent that increases venuleness, or agent that increases HEV phenotype) either alone or in combination with one or more EC targeting molecule can be administered as a pharmaceutical composition to a subject (e.g., a human, such as a human with inflammatory disease or cancer) systemically (e.g., intravenously) or locally (e.g., intratumorally). Such local administrations may restrict the effect of the treatment (e.g., increase or decrease in expression level of genes, increase or decrease in venuleness, increase or decrease in HEV phenotype, increase or decrease in venular differentiation, increase or decrease in inflammation, or increase or decrease in leukocyte recruitment) to the site of administration. For example, intratumoral injection of an agent (e.g., agent that increases venuleness, or agent that increases HEV phenotype) either alone or in combination with one or more EC targeting molecule and/or one or more anti-cancer therapeutic may increase HEV phenotype, venular differentiation and leukocyte recruitment in the tumor microenvironment.

The dosage of the pharmaceutical compositions of the agent (e.g., agent that reduces venuleness, or agent that increases venuleness) either alone or in combination with one or more EC targeting molecule depends on factors including the route of administration, the severity of the condition to be treated, and physical characteristics, e.g., age, weight, general health, of the subject. A pharmaceutical composition may include a dosage ranging from 1 ng/kg to about 100 g/kg (e.g., 1-10 ng/kg, e.g., 2 ng/kg, 3 ng/kg, 4 ng/kg, 5 ng/kg, 6 ng/kg, 7 ng/kg, 8 ng/kg, 9 ng/kg, 10 ng/kg, e.g., 10-100 ng/kg, e.g., 20 ng/kg, 30 ng/kg, 40 ng/kg, 50 ng/kg, 60 ng/kg, 70 ng/kg, 80 ng/kg, 90 ng/kg, 100 ng/kg, e.g., 100-1 μg/kg, e.g., 200 ng/kg, 300 ng/kg, 400 ng/kg, 500 ng/kg, 600 ng/kg, 700 ng/kg, 800 ng/kg, 900 ng/kg, 1 μg/kg, e.g., 1-10 μg/kg, e.g., 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, e.g., 10-100 μg/kg, e.g., 20 μg/kg, 30 μg/kg, 40 μg/kg, 50 μg/kg, 60 μg/kg, 70 μg/kg, 80 μg/kg, 90 μg/kg, 100 μg/kg, e.g., 100-1 mg/kg, e.g., 200 μg/kg, 300 μg/kg, 400 μg/kg, 500 μg/kg, 600 μg/kg, 700 μg/kg, 800 μg/kg, 900 μg/kg, 1 mg/kg, e.g., 1-10 mg/kg, e.g., 2 mg/kg, 3 mg/kg, 4 mg/kg, 5 mg/kg, 6 mg/kg, 7 mg/kg, 8 mg/kg, 9 mg/kg, 10 mg/kg, e.g., 10-100 mg/kg, e.g., 20 mg/kg, 30 mg/kg, 40 mg/kg, 50 mg/kg, 60 mg/kg, 70 mg/kg, 80 mg/kg, 90 mg/kg, 100 mg/kg, e.g., 100-1 g/kg, e.g., 200 mg/kg, 300 mg/kg, 400 mg/kg, 500 mg/kg, 600 mg/kg, 700 mg/kg, 800 mg/kg, 900 mg/kg, 1 g/kg, e.g., 1-10 g/kg, e.g., 2 g/kg, 3 g/kg, 4 g/kg, 5 g/kg, 6 g/kg, 7 g/kg, 8 g/kg, 9 g/kg, 10 g/kg, e.g., 10-100 g/kg, e.g., 20 g/kg, 30 g/kg, 40 g/kg, 50 g/kg, 60 g/kg, 70 g/kg, 80 g/kg, 90 g/kg, 100 g/kg).

The dosage regimen may be determined by the clinical indication being addressed, as well as by various variables (e.g., weight, age, sex of subject) and clinical presentation (e.g., extent or severity of condition). Furthermore, it is understood that all dosages may be continuously given or divided into dosages given per a given time frame. Pharmaceutical compositions that include the agent (e.g., agent that reduces venuleness, agent that reduces HEV phenotype, agent that increases venuleness, or agent that increases HEV phenotype) either alone or in combination with one or more EC targeting molecule of the invention may be administered to a subject in need thereof, for example, one or more times (e.g., 1-10 times or more) daily, weekly, biweekly, monthly, bimonthly, quarterly, biannually, annually, or as medically necessary. Dosages may be provided in either a single or multiple dosage regimens. The timing between administrations may decrease as the medical condition improves or increase as the health of the patient declines.

### VII. Agent Modalities

Agents that can be used for execution of one or more of the methods described herein can be selected from different modalities.

The agent can be a polypeptide (e.g., an inhibitory antibody, an activating antibody, or a targeting antibody), a nucleic acid molecule (e.g., an inhibitory RNA molecule, or an activating RNA molecule), a small molecule (e.g., a small molecule activator, or a small molecule inhibitor), a nuclease (e.g., CAs9), a viral vector (e.g., a lentivirus vector), an overexpression plasmid, or a plasmid encoding an inhibitory, antibody, an activating antigen-binding fragment, an inhibitory protein, and/or an activating peptide.

The agent (e.g., polypeptide, small molecule, or nucleic acid molecule) can be modified. For example, the modification can be a chemical modification (e.g., conjugation to a marker, such as a fluorescent marker or a radioactive marker), conjugation to a molecule that enhances the stability or half-life of the agent, or conjugation to a targeting molecule to target the agent to a particular cell or tissue (e.g., bioconjugation to a targeting antibody to target the agent to a specific tissue). Additionally, the modification can be a packaging modification (e.g., packaging within a nanoparticle or microparticle), or targeting modification to prevent the agent from crossing the blood brain barrier.

### Polypeptides

One or more polypeptides or analogs thereof may be used in the methods described herein.

### Single-Chain Polypeptide

In some embodiments, the polypeptide may be a single-chain polypeptide. Single-chain polypeptides may be used to modulate (increase or decrease) venuleness of ECs by modulating (increasing or decreasing) the expression level of one or more genes listed in Tables 1-68. Agonistic single-chain polypeptides may be used to increase the expression level of one or more of the genes listed in Tables 1-68, and antagonistic single-chain polypeptides may be used to reduce the expression level of one or more of the genes listed in Tables 1-68. Single-chain polypeptides may be in the form of an antibody fragment, such as an antibody fragment known in the art (e.g., a scFv fragment). Single-chain polypeptides may alternatively contain one or more CDRs covalently bound to one another using conventional bond-forming techniques known in the art, for instance, by an amide bond, a thioether bond, a carbon-carbon bond, or by a linker, such as a peptide linker or a linker formed by nucleophilic substitution of a multi-valent electrophile (e.g., a bis(bromomethyl) arene derivative, such as a bis(bromomethyl)benzene or bis(bromomethyl)pyridine) known in the art.

Single-chain polypeptides can be produced by a variety of recombinant and synthetic techniques, such as by recombinant gene expression or solid-phase peptide synthesis procedures known in the art. For instance, one of skill in the art can design polynucleotides encoding, e.g., two or more CDRs operably linked to one another in frame so as to produce a continuous, single-chain peptide containing these CDRs. Optionally, the CDRs may be separated by a spacer, such as by a framework region (e.g., a framework region of a germline consensus sequence of a human antibody) or a flexible linker, such as a poly-glycine or glycine/serine linker known in the art. When produced by chemical synthesis methods, native chemical ligation can optionally be used as a strategy for the synthesis of long peptides (e.g., greater than 50 amino acids). Native chemical ligation protocols are known in the art and have been described, e.g., by Dawson et al. (Science, 266:776-779, 1994); incorporated herein by reference.

### Antibodies

In some embodiments the polypeptide used in the methods described herein is an antibody or antigen-binding fragment thereof. Such antibodies can include polyclonal antibodies, monoclonal antibodies, chimeric antibodies, single-chain antibodies, antibody fragments, humanized antibodies, multispecific antibodies, and modified antibodies (e.g., fused to a protein to facilitate detection). In particular, the antibody may be an inhibitory antibody, an activating antibody, a targeting antibody, or a detection antibody.

(a) Inhibitory antibody: In some embodiments, an inhibitory antibody may be used that is directed against (e.g., binds to) one or more of the genes listed in Tables 1-68. Such an antibody may modulate (e.g., increase or reduce) venuleness of an EC by binding to and reducing the expression level of one or more of the genes listed in Tables 1-68 (e.g., genes having higher expression level in V-ECs compared to NV-ECs, or genes having higher expression level in NV-ECs compared to V-ECs). Specifically, the inhibitory antibody may bind to and reduce the expression level of one or more of the cell surface molecules encoded by the genes listed in Tables 36-68 (e.g., cell surface molecules having higher expression level in V-ECs compared to NV-ECs, or cell surface molecules having higher expression level in NV-ECs compared to V-ECs).

(b) Activating antibody: In some embodiments, an activating antibody may be used that is directed against (e.g., binds to) one or more of the genes listed in Tables 1-68. Such an antibody may modulate (e.g., increase or reduce) venuleness of an EC by binding to and increasing the expression level of one or more of the genes listed in Tables 1-68 (e.g., genes having higher expression level in V-ECs compared to NV-ECs, or genes having higher expression level in NV-ECs compared to V-ECs). Specifically, the activating antibody may bind to and increase the expression level of one or more of the cell surface molecules encoded by the genes listed in Tables 36-68 (e.g., cell surface molecules having higher expression level in V-ECs compared to NV-ECs, or cell surface molecules having higher expression level in NV-ECs compared to V-ECs).

(c) Targeting antibody: In some embodiments, a targeting antibody may be used that is directed against (e.g., binds to) one or more of the genes listed in Tables 69-121. In particular embodiments, a targeting antibody may be an EC targeting molecule. Such an antibody may target ECs in a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by binding to one or more of the genes listed in Tables 69-121 (e.g., genes having higher expression level in ECs in that specific tissue). Specifically, the targeting antibody may target ECs in a specific tissue (e.g., one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter) by binding to one or more of the cell surface molecules encoded by the genes listed in Tables 83-96 or Tables 111-121 (e.g., cell surface molecules having higher expression level in ECs in that specific tissue compared to other tissues).

(d) Detecting antibody: In some embodiments, a detecting antibody may be used for detection of specific properties of cells (e.g., venuleness), or specific types of cells (e.g., a V-EC or NV-EC). Such an antibody may be directed against a protein marker specific to that property (e.g., a marker of venuleness, such as DARC), or a protein marker specific to that kind of cell (e.g., a marker for ECs, such as CD31). One or more detecting antibodies may be used to detect the presence or absence of one or more specific protein markers to distinguish, sort out, identify, or detect specific kind of cells, or specific properties of cells. For example, antibodies directed against DARC and CD31 may be used to sort V-ECs (CD31+DARC+) and NV-ECs (CD31+DARC−) from single cell suspension. Such detecting antibodies may be used in one or more technical platforms (e.g., flow cytometry, western blot, immunohistochemistry, or ELISA). For example, detecting antibody directed against DARC may be used to detect the expression of DARC by both flow cytometry and immunohistochemistry.

In some embodiments, one or more of the polypeptides used in the methods described herein is encoded by a plasmid. For example, an activating polypeptide (e.g., an activating antibody and/or an activating protein), an inhibitory polypeptide (e.g., an inhibitory antibody, an inhibitory antigen-binding fragment, an inhibitory protein, and/or an inhibitory peptide), a targeting polypeptide (e.g., a targeting antibody) and/or a detecting polypeptide (e.g., a detecting antibody) used in the methods described herein can be encoded by a plasmid. In some embodiments, the inhibitory antibody, an inhibitory antigen-binding fragment, inhibitory protein, and/or inhibitory peptide encoded by a plasmid can reduce the expression of the gene products of one or more of the genes listed in Tables 1-68. In some embodiments, the inhibitory antibody, an inhibitory antigen-binding fragment, inhibitory protein, and/or inhibitory peptide encoded by a plasmid can degrade the gene products of one or more of the genes listed in Tables 1-68. In some embodiments, the inhibitory antibody, an inhibitory antigen-binding fragment, inhibitory protein, and/or inhibitory peptide encoded by a plasmid can inhibit the activity and/or function of the gene products of one or more of the genes listed in Tables 1-68. The making and use of antibodies against a target antigen (e.g., protein product of one or more genes listed in Tables 1-81)) is known in the art. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

A pharmaceutical composition comprising the polypeptide can be formulated for treatment of an inflammatory disease and/or cancer in a subject (e.g., a human, such as a human patient in need thereof). In some embodiments, a pharmaceutical composition that includes the polypeptide is formulated for local administration, e.g., to the affected site in a subject.

Methods of making a polypeptide are routine in the art. See, in general, Smales & James (Eds.), Therapeutic Proteins: Methods and Protocols (Methods in Molecular Biology), Humana Press 2005; and Crommelin, Sindelar & Meibohm (Eds.), Pharmaceutical Biotechnology: Fundamentals and Applications, Springer 2013. Some methods for producing a polypeptide involve expression in mammalian cells, although recombinant proteins can also be produced using insect cells, yeast, bacteria, or other cells under the control of appropriate promoters. Mammalian expression vectors may comprise non-transcribed elements such as an origin of replication, a suitable promoter and enhancer, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press 2012.

Various mammalian cell culture systems can be employed to express and manufacture recombinant protein. Examples of mammalian expression systems include CHO cells, COS cells, HeLa and BHK cell lines. Processes of host cell culture for production of protein therapeutics are described in Zhou and Kantardjieff (Eds.), Mammalian Cell Cultures for Biologics Manufacturing (Advances in Biochemical Engineering/Biotechnology), Springer 2014.

### Small Molecules

One or more small molecules may be used in the methods described herein. In particular, the small molecule used in these methods may be a small molecule inhibitor (e.g., a small molecule antagonist), or a small molecule activator (e.g., a small molecule agonist).

(a) Small molecule inhibitor: In some embodiments, a small molecule inhibitor may be used that reduces the expression level of one or more of the genes listed in Tables 1-68. Such a small molecule inhibitor may modulate (e.g., increase or reduce) venuleness of an EC by reducing the expression level of one or more of the genes listed in Tables 1-68 (e.g., genes having higher expression level in V-ECs compared to NV-ECs, or genes having higher expression level in NV-ECs compared to V-ECs).

(b) Small molecule activator: In some embodiments, a small molecule activator may be used that increases the expression level of one or more of the genes listed in Tables 1-68. Such a small molecule activator may modulate (e.g., increase or reduce) venuleness of an EC by increasing the expression level of one or more of the genes listed in Tables 1-68 (e.g., genes having higher expression level in V-ECs compared to NV-ECs, or genes having higher expression level in NV-ECs compared to V-ECs).

Small molecules include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organometallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds.

A pharmaceutical composition comprising one or more of the small molecules can be formulated for treatment of an inflammatory disease and/or cancer in a subject (e.g., a human, such as a human patient in need thereof). In some embodiments, a pharmaceutical composition that includes the small molecule is formulated for local administration, e.g., to the affected site in a subject.

### Nucleic Acids

One or more nucleic acids may be used in the methods described herein. The nucleic acid used in the methods can be a DNA molecule (e.g., a cDNA), a RNA molecule (e.g., an activating RNA molecule (e.g., mRNA, or saRNA), or an inhibitory RNA molecule (e.g., siRNA, shRNA, miRNA, mRNA, or modified mRNA)), or a hybrid DNA-RNA molecule.

### Inhibitory RNA

In some embodiments the nucleic acid agent used in the methods described herein is an inhibitory RNA molecule. In some embodiments, an inhibitory RNA molecule may be used that is directed against one or more of the genes listed in Tables 1-68. Such an inhibitory RNA molecule may modulate (e.g., increase or reduce) venuleness of an EC by reducing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs). An inhibitory RNA molecule may also modulate (e.g., increase or reduce) venuleness of an EC by degrading the gene product of one or more of the genes listed in Tables 1-68 (e.g., genes having higher expression level in V-ECs compared to NV-ECs, or genes having higher expression level in NV-ECs compared to V-ECs). An inhibitory RNA molecule may also modulate (e.g., increase or reduce) venuleness of an EC by inhibiting the activity of the gene product of one or more of the genes listed in Tables 1-68 (e.g., genes having higher expression level in V-ECs compared to NV-ECs, or genes having higher expression level in NV-ECs compared to V-ECs). In particular, an inhibitory RNA molecule may be a short interfering RNA (siRNA), a short hairpin RNA (shRNA), and/or a microRNA (miRNA) that targets one or more of the genes listed in Tables 1-68. An siRNA is a double-stranded RNA molecule that typically has a length of about 19-25 base pairs. A shRNA is a RNA molecule comprising a hairpin turn that decreases expression of target genes via RAN interference (RNAi). shRNAs can be delivered to cells in the form of plasmids, (e.g., viral or bacterial vectors) by transfection, electroporation, or transduction. A microRNA is a non-coding RNA molecule that typically has a length of about 22 nucleotides. miRNAs bind to target sites on mRNA molecules and silence the mRNA, e.g., by causing cleavage of the mRNA, destabilization of the mRNA, or inhibition of translation of the mRNA. In some embodiments, an inhibitory RNA molecule may be a messenger RNA (mRNA) or a modified mRNA that encodes an inhibitory antibody, an inhibitory antigen-binding fragment, inhibitory protein, and/or inhibitory peptide, which targets (e.g., is directed to) one or more of the genes listed in Tables 1-68. In some embodiments, the inhibitory RNA molecule may decrease the level and/or activity of positive regulator of expression and/or function of one or more genes listed in Tables 1-68. In some embodiments, the inhibitory RNA molecule may increase the level and/or activity of an inhibitor of a positive regulator of expression of one or more genes listed in Tables 1-68. In different embodiments, the inhibitory RNA molecule may increase the expression level and/or activity of a negative regulator of expression and/or function of one or more genes listed in Tables 1-68.

An inhibitory RNA molecule can be modified, e.g., to contain modified nucleotides, e.g., 2′-fluoro, 2′-O-methyl, 2′-deoxy, unlocked nucleic acid, locked nucleic acid (2′-O, 4′-C-methylene), 2′-hydroxy, phosphorothioate, 2′-thiouridine, 4′-thiouridine, or 2′-deoxyuridine. Without being bound by theory, it is believed that certain modification can increase nuclease resistance and/or serum stability, or decrease immunogenicity.

In some embodiments, the inhibitory RNA molecule decreases the expression level and/or activity or function of one or more genes listed in Tables 1-68. In some embodiments, the inhibitory RNA molecule inhibits expression of the protein product of one or more genes listed in Tables 1-68 (e.g., inhibits translation to protein). In other embodiments, the inhibitory RNA molecule increases degradation and/or decreases the stability (i.e., half-life) of the protein product of one or more genes listed in Tables 1-68. The inhibitory RNA molecule can be chemically synthesized or transcribed in vitro.

The making and use of inhibitory agents based on non-coding RNA such as ribozymes, RNase P, siRNAs, and miRNAs are also known in the art, for example, as described in Sioud, RNA Therapeutics: Function, Design, and Delivery (Methods in Molecular Biology). Humana Press 2010.

### Activating RNA

In some embodiments the nucleic acid agent used in the methods described herein is an activating RNA molecule. In some embodiments, an activating RNA antibody may be used that is directed against one or more of the genes listed in Tables 1-68. Such an activating RNA molecule may modulate (e.g., increase or reduce) venuleness of an EC by increasing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs). In particular, an activating RNA molecule may be an activating mRNA molecule, or a small activating RNA (saRNA) molecule.

In some embodiments, the activating RNA molecule used in the methods described herein is an activating mRNA molecule, e.g., a synthetic mRNA molecule encoding a protein product of one or more of the genes listed in Tables 1-68. In some embodiments, the activating mRNA molecule may increase the expression level and/or activity of one or more of the genes listed in Tables 1-68. In some embodiments, the activating mRNA molecule may increase the expression level and/or activity of a positive regulator of expression and/or function of one or more genes listed in Tables 1-68. In some embodiments, the activating mRNA molecule may decrease the expression level and/or activity of an inhibitor of a positive regulator of expression and/or function of one or more genes listed in Tables 1-68. In different embodiments, the activating mRNA molecule may decrease the expression level and/or activity of a negative regulator of expression and/or function of one or more genes listed in Tables 1-68.

The mRNA molecule may increase the expression level (e.g., protein and/or mRNA level) and/or activity or function of one or more of the genes listed in Tables 1-68. The mRNA molecule can encode a polypeptide having at least 50% (e.g., at least 50%, 60%, 70%, 80%, 90%, 95%, 97%, 99%, or greater) identity to the amino acid sequence of the protein product of one or more genes listed in Tables 1-68. In other examples, the mRNA molecule has at least 50% (e.g., at least 50%, 60%, 70%, 80%, 90%, 95%, 97%, 99%, or greater) identity to the nucleic acid sequence of one or more genes listed in Tables 1-68. The mRNA molecule can encode an amino acid sequence differing by no more than 30 (e.g., no more than 30, 20, 10, 5, 4, 3, 2, or 1) amino acids to the amino acid sequence of the protein product of one or more genes listed in Tables 1-68. The mRNA molecule can have a sequence encoding a fragment of the protein product of one or more genes listed in Tables 1-68. For example, the fragment comprises 10-20, 20-40, 40-60, 60-80, 80-100, 100-120, 120-140, 140-160, 160-180, 180-200, 200-250, 250-300, 300-400, 400-500, 500-600, or more amino acids in length. In some embodiments, the fragment is a functional fragment, e.g., having at least 20%, e.g., at least 20%, 30%, 40%, 50%, 60%, 70%, 80%, 90%, or greater, of an activity of a full-length protein product of one or more genes listed in Tables 1-68. In some embodiments, the mRNA molecule increases the level and/or activity or function of or encodes the protein product of one or more genes listed in Tables 1-68.

In some embodiments the activating RNA molecule used in the methods described herein is an saRNA molecule. saRNAs are small double-stranded RNAs (dsRNAs) that target gene promoters to induce transcriptional gene activation (e.g., transcriptional activation of one or more genes listed in Tables 1-68) in a process known as RNA activation (RNAa). saRNAs are typically 21 nucleotides in length with 2 nucleotides overhang at the 3′ end of each strand, the same structure of a typical siRNA. To identify an saRNA that can activate a gene of interest, several saRNAs need to be designed within a 1- to 2-kb promoter region by following a set of rules and tested in cultured cells. In some reports, saRNAs are designed in such a way to target non-coding transcripts that overlap the promoter sequence of a protein coding gene. Both chemically synthesized saRNAs and saRNAs expressed as shRNA have been used in in vitro and in vivo experiments.

In some embodiments, the saRNA molecule may increase the expression level and/or activity of one or more of the genes listed in Tables 1-68. In some embodiments, the saRNA used in the methods described herein may increase the expression level and/or activity of a positive regulator of expression and/or function of one or more genes listed in Tables 1-68. In some embodiments, the saRNA molecule may decrease the expression level and/or activity of an inhibitor of a positive regulator of expression and/or function of one or more genes listed in Tables 1-68. In different embodiments, the saRNA molecule may decrease the expression level and/or activity of a negative regulator of expression and/or function of one or more genes listed in Tables 1-68.

The synthetic mRNA molecule can be modified, e.g., chemically. The mRNA molecule can be chemically synthesized or transcribed in vitro. The mRNA molecule can be disposed on a plasmid, e.g., a viral vector, bacterial vector, or eukaryotic expression vector. In some examples, the mRNA molecule can be delivered to cells by transfection, electroporation, or transduction (e.g., adenoviral or lentiviral transduction).

In some embodiments, the modified RNA encoding the protein product of one or more genes listed in Tables 1-68 has modified nucleosides or nucleotides. Such modifications are known and are described, e.g., in WO2012019168. Additional modifications are described, e.g., in WO2015038892; WO2015038892; WO2015089511; WO2015196130; WO2015196118 and WO2015196128A2.

In some embodiments, the modified RNA encoding a polypeptide of interest described herein has one or more terminal modifications, e.g., a 5′Cap structure and/or a poly-A tail (e.g., of between 100-200 nucleotides in length). The 5′ cap structure may be selected from the group consisting of CapO, CapI, ARCA, inosine, NI-methyl-guanosine, 2′fluoro-guanosine, 7-deaza-guanosine, 8-oxo-guanosine, 2-amino-guanosine, LNA-guanosine, and 2-azido-guanosine. In some cases, the modified RNAs also contain a 5′ UTR comprising at least one Kozak sequence, and a 3′ UTR. Such modifications are known and are described, e.g., in WO2012135805 and WO2013052523. Additional terminal modifications are described, e.g., in WO2014164253 and WO2016011306. WO2012045075 and WO2014093924

Chimeric enzymes for synthesizing capped RNA molecules (e.g., modified mRNA) which may include at least one chemical modification are described in WO2014028429.

In some embodiments, a modified mRNA may be cyclized, or concatemerized, to generate a translation competent molecule to assist interactions between poly-A binding proteins and 5′-end binding proteins. The mechanism of cyclization or concatemerization may occur through at least 3 different routes: 1) chemical, 2) enzymatic, and 3) ribozyme catalyzed. The newly formed 5′-/3′-linkage may be intramolecular or intermolecular. Such modifications are described, e.g., in WO2013151736.

Methods of making and purifying modified RNAs are known and disclosed in the art. For example, modified RNAs are made using only in vitro transcription (IVT) enzymatic synthesis. Methods of making IVT polynucleotides are known in the art and are described in WO2013151666, WO2013151668, WO2013151663, WO2013151669, WO2013151670, WO2013151664, WO2013151665, WO2013151671, WO2013151672, WO2013151667 and WO2013151736.S Methods of purification include purifying an RNA transcript comprising a polyA tail by contacting the sample with a surface linked to a plurality of thymidines or derivatives thereof and/or a plurality of uracils or derivatives thereof (polyT/U) under conditions such that the RNA transcript binds to the surface and eluting the purified RNA transcript from the surface (WO2014152031); using ion (e.g., anion) exchange chromatography that allows for separation of longer RNAs up to 10,000 nucleotides in length via a scalable method (WO2014144767); and subjecting a modified mRNA sample to DNase treatment (WO2014152030).

Formulations of modified RNAs are known and are described, e.g., in WO2013090648. For example, the formulation may be, but is not limited to, nanoparticles, poly(lactic-co-glycolic acid)(PLGA) microspheres, lipidoids, lipoplex, liposome, polymers, carbohydrates (including simple sugars), cationic lipids, fibrin gel, fibrin hydrogel, fibrin glue, fibrin sealant, fibrinogen, thrombin, rapidly eliminated lipid nanoparticles (reLNPs) and combinations thereof.

Modified RNAs encoding polypeptides in the fields of human disease, antibodies, viruses, and a variety of in vivo settings are known and are disclosed in for example, Table 6 of International Publication Nos. WO2013151666, WO2013151668, WO2013151663, WO2013151669, WO2013151670, WO2013151664, WO2013151665, and WO2013151736; Tables 6 and 7 of International Publication No. WO2013151672; Tables 6, 178 and 179 of International Publication No. WO2013151671; Tables 6, 185 and 186 of International Publication No. WO2013151667. Any of the foregoing may be synthesized as an IVT polynucleotide, chimeric polynucleotide or a circular polynucleotide, and each may comprise one or more modified nucleotides or terminal modifications.

### Nucleases

One or more nucleases may be used in the methods described herein. In some embodiments, a nuclease may be used that is directed against one or more of the genes listed in Tables 1-68. Such a nuclease may modulate (e.g., increase or reduce) venuleness of an EC by: (i) reducing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs); or (ii) increasing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs). In particular, a nuclease used in the methods described herein may be a zinc finger nuclease (ZFN), a transcription activator-like effector-based nuclease (TALEN), or a Cas9. Such a nuclease may increase and/or decrease the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs) by one or more of the gene editing techniques.

### Gene Editing

In some embodiments, the nuclease used in the methods described herein may be a component of a gene editing system. For example, the nuclease may introduce an alteration (e.g., insertion, deletion (e.g., knockout), translocation, inversion, single point mutation, or other mutation) in one or more genes listed in Tables 1-68. Exemplary gene editing systems that include ZFN, TALEN, or Cas (CRISPR-based methods) are described, e.g., in Gaj et al. Trends Biotechnol. 31.7(2013):397-405.

CRISPR refers to a set of (or system comprising a set of) clustered regularly interspaced short palindromic repeats. A CRISPR system refers to a system derived from CRISPR and Cas (a CRISPR-associated protein) or other nuclease that can be used to silence or mutate a gene described herein. The CRISPR system is a naturally occurring system found in bacterial and archeal genomes. The CRISPR locus is made up of alternating repeat and spacer sequences. In naturally-occurring CRISPR systems, the spacers are typically sequences that are foreign to the bacterium (e.g., plasmid or phage sequences). The CRISPR system has been modified for use in gene editing (e.g., changing, silencing, and/or enhancing certain genes) in eukaryotes. See, e.g., Wiedenheft et al., Nature 482: 331, 2012. For example, such modification of the system includes introducing into a eukaryotic cell a plasmid containing a specifically-designed CRISPR and one or more appropriate Cas proteins. The CRISPR locus is transcribed into RNA and processed by Cas proteins into small RNAs that comprise a repeat sequence flanked by a spacer. The RNAs serve as guides to direct Cas proteins to silence specific DNA/RNA sequences, depending on the spacer sequence. See, e.g., Horvath et al., Science 327: 167, 2010; Makarova et al., Biology Direct 1:7, 2006; Pennisi, Science 341: 833, 2013. In some examples, the CRISPR system includes the Cas9 protein, a nuclease that cuts on both strands of the DNA. See, e.g., i.d.

In some embodiments, in a CRISPR system for use described herein, e.g., in accordance with one or more methods described herein, the spacers of the CRISPR are derived from a target gene sequence, e.g., from a sequence of one or more genes listed in Tables 1-68.

In some embodiments, the methods described herein may involve the use of a guide RNA (gRNA) for use in a clustered regulatory interspaced short palindromic repeat (CRISPR) system for gene editing. In some embodiments, the nuclease used in the methods described herein is a ZFN, or an mRNA encoding a ZFN, that targets (e.g., cleaves) a nucleic acid sequence (e.g., DNA sequence) of one or more genes listed in Tables 1-68. In some embodiments, the nuclease used in the methods described herein is a TALEN, or an mRNA encoding a TALEN, that targets (e.g., cleaves) a nucleic acid sequence (e.g., DNA sequence) in one or more genes listed in Tables 1-68.

For example, the gRNA can be used in a CRISPR system to engineer an alteration in a gene (e.g., one or more genes listed in Tables 1-68). In other examples, the ZFN and/or TALEN can be used to engineer an alteration in a gene (e.g., one or more genes listed in Tables 1-68). Exemplary alterations include insertions, deletions (e.g., knockouts), translocations, inversions, single point mutations, or other mutations. The alteration can be introduced in the gene in a cell, e.g., in vitro, ex vivo, or in vivo. In some examples, the alteration may increase the expression level and/or activity of one or more genes listed in Tables 1-68, or the protein product of one or more genes listed in Tables 1-68. In other examples, the alteration decreases the expression level and/or activity of (e.g., knocks down or knocks out) one or more genes listed in Tables 1-68. In yet another example, the alteration corrects a defect (e.g., a mutation causing a defect), in one or more genes listed in Tables 1-68.

In certain embodiments, the CRISPR system is used to edit (e.g., to add or delete a base pair) a target gene, e.g., one or more genes listed in Tables 1-68. In other embodiments, the CRISPR system is used to introduce a premature stop codon, e.g., thereby decreasing the expression of a target gene. In yet other embodiments, the CRISPR system is used to turn off a target gene in a reversible manner, e.g., similarly to RNA interference. In some embodiments, the CRISPR system is used to direct Cas (e.g., Cas 9) to a promoter of one or more genes listed in Tables 1-68, thereby blocking an RNA polymerase sterically.

In some embodiments, a CRISPR system can be generated to edit a gene (e.g., one or more genes listed in Tables 1-68), using technology described in, e.g., U.S. Publication No. 20140068797; Cong, Science 339: 819, 2013; Tsai, Nature Biotechnol., 32:569, 2014; and U.S. Pat. Nos. 8,871,445; 8,865,406; 8,795,965; 8,771,945; and 8,697,359.

In some embodiments, the CRISPR interference (CRISPRi) technique can be used for transcriptional repression of specific genes, e.g., one or more genes listed in Tables 1-68). In CRISPRi, an engineered Cas9 protein (e.g., nuclease-null dCas9, or dCas9 fusion protein, e.g., dCas9-KRAB or dCas9-SID4X fusion) can pair with a sequence specific guide RNA (sgRNA). The Cas9-gRNA complex can block RNA polymerase, thereby interfering with transcription elongation. The complex can also block transcription initiation by interfering with transcription factor binding. The CRISPRi method is specific with minimal off-target effects and is multiplexable, e.g., can simultaneously repress more than one gene (e.g., using multiple gRNAs). Also, the CRISPRi method permits reversible gene repression.

In some embodiments, CRISPR-mediated gene activation (CRISPRa) can be used for transcriptional activation, e.g., of one or more genes listed in Tables 1-68). In the CRISPRa technique, dCas9 fusion proteins recruit transcriptional activators. For example, dCas9 can be used to recruit polypeptides (e.g., activation domains) such as VP64 or the p65 activation domain (p65D) and used with sgRNA (e.g., a single sgRNA or multiple sgRNAs), to activate a gene or genes, e.g., endogenous gene(s). Multiple activators can be recruited by using multiple sgRNAs—this can increase activation efficiency. A variety of activation domains and single or multiple activation domains can be used. In addition to engineering dCas9 to recruit activators, sgRNAs can also be engineered to recruit activators. For example, RNA aptamers can be incorporated into a sgRNA to recruit proteins (e.g., activation domains) such as VP64. In some examples, the synergistic activation mediator (SAM) system can be used for transcriptional activation. In SAM, MS2 aptamers are added to the sgRNA. MS2 recruits the MS2 coat protein (MCP) fused to p65AD and heat shock factor 1 (HSF1).

The CRISPRi and CRISPRa techniques are described in greater detail, e.g., in Dominguez et al., Nat. Rev. Mol. Cell Biol. 17:5, 2016, incorporated herein by reference.

### Viral Vectors

In some embodiments, the agent used to execute the methods described herein may be a viral vector. Viral vectors can be used to express a transgene encoding one or more genes listed in Tables 1-68. A viral vector may be administered to a cell or to a subject (e.g., a human subject or animal model) to increase expression of one or more genes listed in Tables 1-68. A viral vector may also be administered to a cell or to a subject (e.g., a human subject or animal model) to reduce expression of one or more genes listed in Tables 1-68. A viral vector may modulate (e.g., increase or reduce) venuleness of an EC by: (i) reducing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs); or (ii) increasing the expression level (e.g., protein level or mRNA level) of one or more of the genes listed in Tables 1-68 (e.g., genes having higher expression level in V-ECs compared to NV-ECs, or genes having higher expression level in NV-ECs compared to V-ECs).

Viral genomes provide a rich source of vectors that can be used for the efficient delivery of exogenous genes into a mammalian cell. Viral genomes are particularly useful vectors for gene delivery because the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

### EXAMPLES

The following examples are put forth so as to provide those of ordinary skill in the art with a description of how the methods described herein may be used and evaluated, and are intended to be purely exemplary of the invention and are not intended to limit the scope of what the inventors regard as their invention.

### Example 1. Identification of Genes Differentially Expressed by V-ECs or NV-ECs

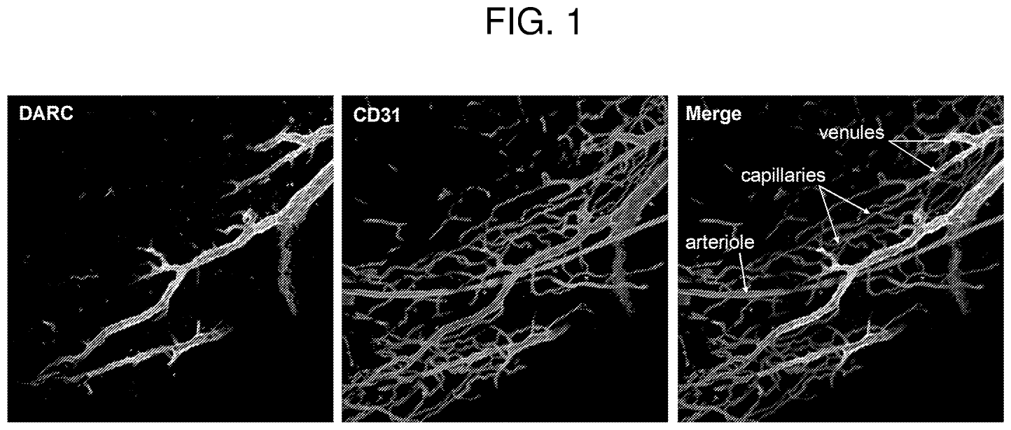

The invention discloses lists of genes that are differentially expressed by V-ECs or NV-ECs (e.g., higher expression level in V-ECs compared to NV-ECs, or higher expression level in NV-ECs compared to V-ECs) in fourteen different murine tissues (e.g., peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter) that can be used to identify potential targets for anti-inflammatory therapy and/or cancer therapy (Tables 1-68). A discovery strategy was devised that allowed comprehensive transcriptome analyses of freshly purified V-ECs and NV-ECs from virtually any vascularized tissue. To this end, a non-signaling chemokine binding receptor, DARC (Duffy Antigen/Receptor for Chemokines), which had been suggested to be a specific marker for venular ECs in humans was used. The first monoclonal antibody (mAb) that recognizes the erythroid and endothelial forms of murine DARC was generated as described by Thiriot et al. (BMC Biol 15:45, 2017), included herein by reference. The DARC mAb could be used for detection of DARC expression in murine tissues by both flow cytometry and immunohistochemistry (IHC). An IHC micrograph demonstrating venule-restricted DARC expression in a whole-mount preparation of mouse omentum is shown in FIG. 1. Using this approach, DARC expression was determined to be highly restricted to venules in every tissue in the body. Taking advantage of this new tool, venular (CD31+DARC+) ECs (V-ECs), non-venular (CD31+DARC−) ECs (NV-ECs), and lymphatic ECs (LECs) from single-cell suspensions of multiple murine tissues were sorted by flow cytometry, and their transcriptomes were determined by RNA sequencing analysis (SMART-Seq2M, Nextera XT). Datasets were obtained for peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. The information gleaned from the analysis allows identification of genes that are selectively expressed in venules, either globally or in an organ-restricted fashion.

Bioinformatic analysis of the RNA sequencing data was performed as followed. The paired Fastq sequences of length 25 bp for each sample were aligned to the mouse genome (Gencode version M16) using STAR version 2.0.6c. Reads aligning with gene exons were counted using the feature Counts function of the Subread package version 1.6.2. Collapsing of technical replicates and normalization was performed using DESeq2 version 1.20.0. Differential gene expressions were performed by DESeq2 to compare V-ECs to NV-ECs samples for a given tissue. Results were filtered for fold change of 1.9 and adjusted P-value of 0.05. Samples failing any of the quality control steps (e.g., read counts, gene body coverage, and pairwise correlation) were excluded from the analysis, as described in Dobin et al. (Bioinformatics 29:15, 2013), Liao et al. (Bioinformatics 30:923, 2013), and Love et al. (Genome Biol 15:550, 2014).

The results of analysis of genes having higher expression level in V-ECs compared to NV-ECs or higher expression level in NV-ECs compared to V-ECs in peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and/or dura matter are shown in Tables 1-68. Table 1 lists genes whose expression level is higher (fold change ≥1.9) in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, and dura matter, allowing no exception (tolerate 0). Table 2 lists genes whose expression level is higher (fold change ≥1.9) in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, and dura matter, allowing one exception (tolerate 1). Table 3 lists genes whose expression level is higher (fold change ≥1.9) in V-ECs compared to NV-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter, allowing three exceptions (tolerate 3). Tables 4-16 list genes whose expression level is higher (fold change ≥1.9) in V-ECs compared to NV-ECs in one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Table 17 lists genes whose expression level is higher (fold change ≥1.9) in V-ECs compared to NV-ECs in all of peripheral lymph node, mesenteric lymph node, and Peyer's patch, i.e., genes whose expression level is higher (fold change ≥1.9) in HEVs, compared to thymus, visceral adipose tissue, small intestine, colon, subcutaneous adipose tissue, bone tissue, uterus, brain parenchyma, choroid plexus, pia matter, and dura matter. Table 18 lists Madcam1 and Slco3a1 as the only gut V-EC specific genes, i.e., a gene whose expression level is higher (fold change ≥1.9) in V-ECs compared to NV-ECs in all of mesenteric lymph node, Peyer's patch, small intestine, and colon compared to peripheral lymph node, thymus, visceral adipose tissue, small intestine, colon, subcutaneous adipose tissue, bone tissue, uterus, brain parenchyma, choroid plexus, pia matter, and dura matter. Table 19 lists genes whose expression level is higher (fold change ≥1.9) in NV-ECs compared to V-ECs (i.e., lower in V-ECs compared to NV-ECs) in all of peripheral lymph node, mesenteric lymph node, Peyer's patch, thymus, visceral adipose tissue, small intestine, colon, subcutaneous adipose tissue, bone tissue, uterus and dura matter, allowing no exception (tolerate 0). Table 20 lists genes whose expression level is higher (fold change ≥1.9) in NV-ECs compared to V-ECs (i.e., lower in V-ECs compared to NV-ECs) in all of peripheral lymph node, mesenteric lymph node, Peyer's patch, thymus, visceral adipose tissue, small intestine, colon, subcutaneous adipose tissue, bone tissue, uterus, and dura matter, allowing one exception (tolerate 1). Table 21 lists genes whose expression level is higher (fold change ≥1.9) in NV-ECs compared to V-ECs in all of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter, allowing three exceptions (tolerate 3). Tables 22-35 list genes whose expression level is higher (fold change ≥1.9) in NV-ECs compared to V-ECs (i.e., lower in V-ECs compared to NV-ECs) in one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, and dura matter. Tables 36-51 list the cell surface molecule genes respectively from Tables 1-16. Table 52 lists the cell surface molecule genes from Table 17. Tables 53-68 list the cell surface molecule genes respectively from Tables 19-20 and 22-35.

TABLE 1
Genes with higher expression level in V-ECs compared to NV-ECs in
peripheral lymph node, mesenteric lymph node, Peyer's patch, thymus,
visceral adipose tissue, small intestine, colon, subcutaneous adipose
tissue, bone tissue, uterus and dura matter (pan-venular, tolerate 0,
7 genes)
Gene name
Ackr1*, Lrg1, Plekha7, Dennd2d, Spint2*, Pdia5, Nr2f2

TABLE 2
Genes with higher expression level in V-ECs compared to NV-ECs in
peripheral lymph node, mesenteric lymph node, Peyer's patch, thymus,
visceral adipose tissue, small intestine, colon, subcutaneous adipose
tissue, bone tissue, uterus, and dura matter (pan-venular, tolerate 1, 32
genes)
Gene name
Ackr1*, Itih3, Cadm3*, Adgrg6*, Rasgef1a, Gm38070, Pcdh7*, Lrg1,
Sirpa*, Ehd3, Tgfbi, Vcam1*, Plekha7, Dennd2d, Ctsh, Lepr*, Tll1,
Spint2*, Pdia5, Cd55, Bend4, Nr2f2, Il1r1*, Acer3, Kank1, Tbc1d8,
Net1, Zfp521, Icam1*, Vav2, Hid1, Psen2*

TABLE 3
Genes with higher expression level in V-ECs compared to NV-ECs in
peripheral lymph node, mesenteric lymph node, Peyer's patch, thymus,
visceral adipose tissue, small intestine, colon, subcutaneous adipose
tissue, bone tissue, uterus, brain parenchyma, choroid plexus, pia and
dura matter (pan-venular, tolerate 3, 42 genes)
Gene name
Ackr1*, Sele*, Selp*, Itih3, Cadm3*, Adgrg6*, Gm38070, Lrg1,
Rasgef1a, Lbp, Ptk2b, Ch25h, Vcam1*, Pcdh7*, Dennd2d, Ctsh,
Plekha7, Myof*, Tll1, Thsd7a*, Spint2*, Cd55, Pdia5, Gem, Rcan1,
Nr2f2, Il1r1*, Procr*, Traf1, Prkag2, Rab27a, Icam1*, Kank1, Net1,
Acer3, Il6st*, Tbc1d8, Vav2, Zfp521, Hid1, Psen2*, Fth1

TABLE 4
Genes with higher expression level in V-ECs compared to NV-ECs in
peripheral lymph node (37 genes)
Gene name
Gpc1, Rprm, Ttn, Dclk1*, Gm42608, Gm38244, 2010016l18Rik,
Gm42510, 6030400A10Rik, Gm42635, Gm42480, Gm42482, Gabra2*,
Cfap73, Gm10382, Rasgef1a, Apoe, Rps3, Ccnd1, Csmd1,
8030455M16Rik, Dapk3, Atcay, Gm48372, Gm47472, Fam19a2, Nefh,
Ccdc88a, Gm16170, Gas7, Serpina1b, Tgfbi, Cacna2d3, Ly6i, Glycam1,
AC171205.1, Kcng3*

TABLE 5
Genes with higher expression level in V-ECs compared to NV-ECs in
mesenteric lymph node (9 genes)
Gene name
Aff3, Lrtm2, Tcerg1l, Ces2g, Susd2*, Spic, Cyp2ab1, Hs3st6, Ankrd1

TABLE 6
Genes with higher expression level in V-ECs compared to NV-ECs in
Peyer's patch (79 genes)
Gene name
Trpa1*, Cyp27a1, Scg2, Gbx2, D130058E05Rik, Astn1*, Pappa2,
Kcnh1*, Gcg, Neurod1, Fjx1, Chgb, Snap25, Pcsk2, Snhg11, Kcnq2*,
Tm4sf4, Reg4, Syt6*, Pitx2, Clca1*, Kif12, Pcsk9, Elavl4, Dpp6*,
Dpysl5, Slit2, Slc10a4*, Cwh43, Afp, B230112J18Rik, Hepacam2,
Tac1, Nap1l5, Atoh1, Cd177*, Ceacam10, Tph1, Gabrb3*, Slco3a1*,
Ap3b2, Ddias, Zg16, Sct, Muc2, Slc18a1, Gm5159, Chrna3*, Stra6*,
Celf6, Scg3, Entpd3*, Cck, Vip, Madcam1*, Adcy1*, Fstl4, Pirt*, Pyy,
Rtn1, Chga, Aspg, Gm30238, Gng4, Pcsk1, Rab3c, Gm26660, Slc18a3*,
Adam28*, Sult4a1, Prph, Faim2*, Vwa5b2, Syngr3, Slc35g1*, Pcskin,
Zcchc12, Adgrg4*, Bex2

TABLE 7
Genes with higher expression level in V-ECs compared to NV-ECs
thymus (54 genes)
Gene name
Gpr1*, Tor1aip2, Pfkfb3, Nek6, Gabpb1, Lrrn4*, Car13, Gm8388, Fgg,
Glipr2, Fndc5*, Fuca1, Mul1, Tnfrsf9*, Gm5864, Sod3, Gbp6, Gbp11,
Upk3b*, Tpi1, Fgf23, 2200002D01Rik, Ppp1r14a, Bag3, Tnfrsf23,
Angpt2, Tmem123, Birc3, St3gal4, Hexa, Pik3r4, Fbxo30, Tnfaip3, Pfkl,
Upp1, Gm525, Csf3, H3f3b, Egln3, Hif1a, Ltbp2, Spock1, Upk1b*,
Kcnj15*, Tap1, Map3k8, Vps37c, Vldlr*, Ppp1r3c, Avpi1, Mid1ip1,
Il13ra1, Pgk1, Zfp711

TABLE 8
Genes with higher expression level in V-ECs compared to NV-ECs in
visceral adipose tissue (23 genes)
Gene name
Gm43621, Kcnb1*, Them5, Chil5, Gm42949, Eln, Ret*, Adamtsl3,
Camsap3, Rpl30-ps2, Kirrel3*, Gm38217, Gm48743, Gm48751,
Gm9930, Gm48755, Wnt9b, Gm40663, Adarb2, Gm47118, Masp1,
Rnf39, Snx24

TABLE 9
Genes with higher expression level in V-ECs compared to NV-ECs in
small intestine (363 genes)
Gene name
Il17a, Neurl3, Gm15832, Il1r2*, Gm16894, Gm37347, Gm3940, Icos*,
Vil1, Alpi, Akp3, Arl4c, Rab17, Lct*, AA986860, Il10, Pm20d1,
Ppfia4, Elf3, Rgs1, Sec16b, Fmo4, Fcrla, Fcgr4*, Itln1, Slamf6*, Igsf9*,
Mptx2, Bpnt1, Malrd1, Entpd8*, Slc27a4*, Dnm1, Cytip, Spi1, Depdc7,
Slc5a12*, Ppp1r14d, Ckmt1, Il1a, Il1b, Hck, Ocstamp, Hnf4g, Chmp4c,
Gm5150, Slc2a2*, P2ry13*, Sis, Rbm46, Lrat*, Fcrl1*, Pklr, Tuft1,
Cgn, Ctss, Fcgr1*, Pdzk1, Eps8l3, Gm43745, Gstm3, Dpyd, Fabp2,
Enpep*, Gimd1, Bank1, Mttp, Adh6a, Gbp2b, Clca4b*, Rragd, Car9*,
Aldh1b1, Stra6l*, Skint6, Guca2b, Edn2, Csf3r*, Tlr12*, Tmem54,
Crybg2, Runx3, C1qb, C1qc, C1qa, Pla2g2d, Rnf186, Tmem82,
Slc2a5*, Slc2a7*, Cnpy1, Cgref1, Slc5a1*, Ppargc1a, Slc34a2*,
Gm43627, 9130230L23Rik, Stap1, Ugt2a3, Sult1b1, Cxcl2, Sowahb,
Prkg2, Cds1, Abcg3*, Vps37b, Abhd11os, Mixipl, Ccl24, Pilra*, Pilrb2,
Cyp3a13, Nxpe5, Adap1, Card11, Cyp3a25, Flt3*, Slc13a1*, Clec5a*,
Mgam*, Gpnmb*, Mpp6*, Hpgds, Fabp1, Reg3b, Reg3a, Reg1, Dqx1,
C3ar1*, Clec4a2*, Clec4n, Clec2h*, BC035044, Clec2e*, Clec7a*,
Klra17, Klra2*, Gm19434, Pirb*, Lair1*, Lilra5*, Ptprh*, Igsf23,
Nlrp9b, Erich4, Lgals4, Cd22*, Slc7a9*, Cd33*, Myh14,
0610005C13Rik, Sult2b1, Ush1c, Anpep*, Gm44974, Slc28a1*,
Vmn2r69*, E230029C05Rik, Mogat2, Stard10, Gm8979, Gm1966,
Tmc5*, Slc5a11*, Il21r*, Nlrp6, Ano9*, Rassf7, Cdhr5*, Muc6,
Slc10a2*, Defa30, Defa24, C130073E24Rik, March1*, Lrrc25, Ifi30,
Mmp12, Acp5, Pdzd3, Treh, Apoa1, 2900052N01Rik, Bco2, Slc51b*,
Aph1c*, Pls1, Rbp2, Mst1r*, Slc26a6*, Pfkfb4, Cx3cr1*, Ccr5*,
Zc3h12d, Il22ra2, Gm26740, Smlr1, Calhm6*, Slc16a10*, Gm48065,
Pkib, Pla2g12b, Hkdc1, Pbld2, Ggt1*, Slc5a4a*, Trpm2*, Misp, Reep6,
Creb3l3, Smim24, Gas2l3, Lyz2, Il22, Myo1a, Rdh7, Slc39a5*,
Erbb3*, Gal3st1*, Npc1l1*, Cobl, Wwc1, Fabp6, Havcr2*, Timd4,
Tmem238l, Cd68*, Mgl2, Cxcl16*, Tm4sf5*, Aspa, Evi2a, Wfdc17,
Gm21885, Cwc25, Ccr10*, Nags, Cd300a*, Cd300c2*, Otop3*,
Slc16a5*, Myo15b, Apob, Gpx2, Arg2, Gpr65*, Ifi2712b, Bcl11b,
Amn*, Pld4, Gpr132*, Tubal3, Gpr141*, D130043K22Rik*, Ly86,
Cd83*, Cdhr2*, Tifab, Fbp2, Slc6a19*, Ell2, Gm48302, Iqgap2, Naip5,
Naip6, Tep1, Pnp2, Tmem253, Trac*, Cideb, Gata4, Tnfsf11*,
Slc15a1*, Otulinl, Destamp*, Anxa13, BC025446, Eppk1, Dgat1*, Gpt,
Apol7a, Apol10a, Apol7c, Apol10c-ps, Baiap212, Cyp2d26, Slc38a1*,
Soat2, Mefv, Dnase1, Ccdc116, Clcn2*, Slc51a*, Gm15657, Cd86*,
Nr1i2, Plcxd2, Slc22a1*, Vmn2r102*, Tmem8*, Mapk13, Fgd2,
AC163635.1, Cyp4f40, Cyp4f14, Btnl2*, Btnl5-ps, Btnl6, Btnl7-ps,
Aif1, H2-Q1, H2-Q2, H2-Bl, Gm10499, Gm8909, H2-T3, Trim15,
Trim40, Pla2g7, 1700071M16Rik, Treml4*, Unc5cl, C230085N15Rik,
5830444F18Rik, Adgre4*, Adgre1*, Themis3, Epb41l3, Nlrc4,
Abcg5*, Abcg8*, Slc3a1*, Mep1b*, AC161438.1, Myo7b, Csf1r*,
Myo5b, Cndp1, 2010003K11Rik, Naaladl1*, Tkfc, Cd5*, Cd6*,
Slc15a3, Ms4a18, Ms4a12, Ms4a7, Ms4a4a, Ms4a6c, Mpeg1, Ifit1bl2,
Got1, Abcc2*, Csf2ra*, Glod5, Cybb*, Otc, 5430427O019Rik, Tlr13*,
P2ry10*, Gm6377, Ace2*, Tlr7*

TABLE 10
Genes with higher expression level in V-ECs compared to NV-ECs in
colon (212 genes)
Gene name
Pi15, Il1rl1*, Fhl2, Col3a1, Col5a2, Nab1, Satb2, Catip, Des, Kcne4,
Cdh19*, Gpr161*, Rgs5, Olfm2b, Mptx1, Grem2, Adamts13, Col5a1,
Ncs1, Hmcn2, Lamc3, Zeb2, Tnfaip6, Pla2r1*, Scn7a, Klhl23, Chn1,
Pde1a, Gm13889, Pamr1, Bdnf, Grem1, Actc1, Gpr176*, Fbln7, Rem1,
Jph2*, Cdh26*, Car1, Gm43834, Agtr1b*, Ccdc39, Lhfp, Gm42899,
C130075A20Rik, Postn, Sptssb, Gucy1b1, Gucy1a1, Thbs3, Sprr2a3,
Selenbp1, Hsd3b3, Hao2, Kcna2*, Ank2*, 1500005C15Rik, Clca4a*,
Nexn, St6galnac5, Fhl5, Tpm2, Tnc, 8030451A03Rik, Prkaa2, Podn,
Trabd2b*, Tspan1*, Guca2a, Heyl, Ptafr*, Adam22*, Cacna2d1, Gnai1,
Slc35f6, Spon2, Ablim2, C1qtnf7, Fgfbp1, Ereg*, Parm1*, Arhgap24,
Svopl, Dennd2a, Hoxa11os, Actg2, Pdzrn3, Gm36640, Pde3a, Sox5,
Pla2g4c, Ethe1, 9530053A07Rik, Zfp536, Klk14, Klk10, Plekha4,
Ntrk3*, Cpeb1, Sytl2, Olfr78*, Dkk3, Mical2, Acsm3, Aqp8*, Mrgprf*,
Sfrp1, Pdgfrl, Pdlim3, Gm45407, Ednra*, Hhip*, Tppp3, Gm16208,
Il34, Crispld2, Gm15684, Acta1, Gucy1a2, Trpc6*, Naalad2*, Col5a3,
Cnn1, Adamts15, Nnmt, Ncam1*, Bmp5, Tcf21, Lama2, Calhm5*,
Ggt5*, Col6a2, Col6a1, Fstl3, Ntn4, Mgat4c, Avpr1a*, Gm48878, Gli1,
Itga7*, Mettl7b, 1810065E05Rik, Ccl11, Slfn4, Sstr2*, Alkal2,
Slc26a3*, Dgkb, Akap6, Galnt16, Tgfb3, Meg3, Rtl1, Rian, Gm37899,
B830012L14Rik, Amph, Aspn, Pitx1, Gm15740, Tmem171,
2610528A11 Rik, Ndrg2, Sgcg*, Hr, Gpc6*, C1qtnf3, Gm32618, Sdc2*,
Matn2, Colec10, Gsdmc2, Gsdmc3, Gsdmc4, 2010109103Rik, Cyp2d34,
Cela1, Pla2g10, Myh11, Iglc3, Ephb3*, Retnlb, Abi3bp, Vgll3,
AC098883.2, B3galt5, Tnxb, Tubb4a, Myom1, Dync2li1, Aqp4*,
Garem1, Ppp2r2b, BC023105, Pdgfrb*, Oacyl, Ms4a4d, Gna14,
Mamdc2, Acta2, Lgi1, Cyp2c55, Cpn1, Adra2a*, Usp27x, Slc6a14*,
Gpc3*, Fhl1, Dmd, Nlgn3*, Chrdl1, Gm15261

TABLE 11
Genes with higher expression level in V-ECs compared to NV-ECs in
subcutaneous adipose tissue (14 genes)
Gene name
Mpz*, Ugt8a, Cldn19*, Irs3, Hbb-bs, Comp, Slc36a2*, Kcns3*, Ntrk2*,
Sncg, Ephx2, Mbp, Drp2, Alas2

TABLE 12
Genes with higher expression level in V-ECs compared to NV-ECs in
bone tissue (171 genes)
Gene name
Jph1*, Serpine2, Kif1a, Tmem37, Gm16083, Cdk18, Rgs4, Fcrlb,
Fcgr2b*, Sfmbt2, Mrc1*, Endog, Cd302, Pdk1, Tfpi, Fgf7, Sirpa*,
Gfra4, Snx5, Mafb, Sgk2, Wisp2, Matn4, Cdh4*, Car2, Ptx3, Shox2,
Gpr88*, Adh6b, Ak5, Al115009, Slc26a7*, Gm11837, Ttpa, Aldob,
Snx30, Junos, 4933424M12Rik, Zfp691, Hivep3, Fam213b, Sema3a*,
Gm42676, Gm42675, Gm43719, Crygn, Gm1673, Nat8l, Ibsp, Spp1,
Snx8, Gm15411, 1700028E10Rik, Gm28719, Mgst1*, Kcnj8*, Abcc9*,
C5ar2*, C5ar1*, Rcn3, Gm2788, Kif7, Pex11a, Myo7a, Galnt18,
Ifitm10*, Gm6213, Gm5345, Wdr17, Gpm6a*, Gm45264, Tmem221,
4921524J17Rik, Cdh11*, Hp, Maf, Agt, Epor*, Slc37a2, Clmp*,
Hmgcll1, Paqr9*, Trf, Slc22a14*, Vta1, Dse, Mett124, Oit3, Lrrc3,
1500009L16Rik, Gm16268, Stab2*, Gm16280, Gm16270, Gm16271,
Plxnc1*, Wif1, Rdh16, Ap1b1, Flt4*, Ltc4s, Gpx3, Ccdc69, Rtn4rl1*,
Dhrs13, Tmem132e, Igfbp4, Tmem106a, Engase, Sectm1a*, L2hgdh,
Cdkl1, 4930512B01Rik, Map4k5, Ston2, Gm8378, Lgmn, Serpina3c,
Serpina3i, Epdr1, Sfrp4, Mylip, Gmpr, Unc5a*, Cxcl14, Adcy2*, Irx2,
Gm32067, Map1b, 5330431K02Rik, Naip2, Fst, Tmem273, Vstm4*,
Ptger2*, Rnase10, Loxl2, Ccdc122, Cldn10*, Selenop, Rspo2, Pdzrn4,
Kng2, Kng1, Adipoq, B630019A10Rik, Gm6630, P3h2, Osbpl11,
Zbtb11os1, Cxadr*, Adamts5, Mcrip2, Rab11fip3, Ly6g6e, H2-Q10*,
Prob1, Ccbe1, Lipg, Rhod, Glis3, Slc16a12*, Map7d3, F8a, Gdpd2*,
Mum1l1, Morc4, Tmem164, Il13ra2, Gpr143*, Tlr8*

TABLE 13
Genes with higher expression level in V-ECs compared to NV-ECs in
uterus (31 genes)
Gene name
Gm37265, Pid1, Pax8, Wfdc2, Pdgfc, Atp6v0d2, Tlr4*, Bnc2,
Adamtsl1, P2rx2*, 5930412G12Rik, Kcna6*, Emp1*, Rerg, Bcat1,
Izumo1*, Insr*, Gm16180, Nek5, 3110039108Rik, Gm48742,
Gm48740, Sbno2, Ypel2, Gm4740, Gm49173, Gm17546, AC134560.1,
Prss28, Prss29, Pcdhb2*

TABLE 14
Genes with higher expression level in V-ECs compared to NV-ECs in
brain parenchyma (6 genes)
Gene name
Ryr3*, Coro2a, Lao1, Hif3a, Gm35190, Rims2

TABLE 15
Genes with higher expression level in V-ECs compared to NV-ECs in
pia matter (3 genes)
Gene name
Lrrtm1*, Gm6614*, 5830468F06Rik

TABLE 16
Genes with higher expression level in V-ECs compared to NV-ECs in
dura matter (3 genes)
Gene name
Gm22816, Prr15, Il17ra*

TABLE 17
Genes with higher expression level in V-ECs compared to NV-ECs in
peripheral lymph node, mesenteric lymph node, and Peyer's patch
compared to thymus, VAT, SI, colon, SAT, bone/BM, uterus, brain
parenchyma, choroid plexus, pia and dura matter (HEV specific, 38
genes)
Gene name
Glycam1, Chst4, 2010016l18Rik, Gm38244, Oit1, Itih3, Robo2*, Nefh,
Bpifb5, Serpina3n, Serpina1e, Dusp27, Sspo, Fut7, Gm32742, Hs3st6,
Esrrg, Ccdc33, Btbd11, Sh3gl3, C1rl, Gcnt1, Chst1, Relt*, Lpcat4,
Gm45871, Serpina1b, Susd2*, Man1a, Abca2*, Cpeb2, B4galt6, Xxylt1,
Tmx3*, Srgap3, Il2rg*, Serpinb9, Tarbp1

TABLE 18
Genes with higher expression level in V-ECs compared to NV-ECs in
mesenteric lymph node, Peyer's patch, small intestine, and colon
compared to peripheral lymph node, thymus, visceral adipose tissue,
subcutaneous adipose tissue, bone tissue, uterus, brain parenchyma,
choroid plexus, pia and dura matter (gut V-EC specific, 2 genes)
Gene name
Madcam1*, Slco3a1*

TABLE 19
Genes with higher expression level in NV-ECs compared to V-ECs in
peripheral lymph node, mesenteric lymph node, Peyer's patch, thymus,
visceral adipose tissue, small intestine, colon, subcutaneous adipose
tissue, bone tissue, uterus and dura matter (pan-non-venular, tolerate 0,
4 genes)
Gene name
Notch4*, Tril, Cables2, Pik3r3

TABLE 20
Genes with higher expression level in NV-ECs compared to V-ECs in
peripheral lymph node, mesenteric lymph node, Peyer's patch, thymus,
visceral adipose tissue, small intestine, colon, subcutaneous adipose
tissue, bone tissue, uterus and dura matter (pan-non-venular, tolerate
1, 15 genes)
Gene name
Car7, Clic5, Nid2, Efnb2*, Unc119b, Notch4*, Tril, Cables2, Prdm16,
Kctd12b, Sh3tc2, Rps6ka2, Lamb1, Pik3r3, Rapgef1

TABLE 21
Genes with higher expression level in NV-ECs compared to V-ECs in
peripheral lymph node, mesenteric lymph node, Peyer's patch, thymus,
visceral adipose tissue, small intestine, colon, subcutaneous adipose
tissue, bone tissue, uterus, brain parenchyma, choroid plexus, pia and
dura matter (pan-non-venular, tolerate 3, 25 genes)
Gene name
Igfbp3, Hey1, Rsad2, Rgcc, Ednrb*, Clic5, Sipa1l2, Cxcl12, Nid2,
Efnb2*, Unc119b, Notch4*, Mcc, Tril, Dll4*, Cables2, Prdm16,
Map4k2, Kctd12b, Jup, Lgals3bp, Rps6ka2, Sh3tc2, Pik3r3, Dgkd

TABLE 22
Genes with higher expression level in NV-ECs compared to V-ECs in
peripheral lymph node (5 genes)
Gene name
Creg2, Adam33, Brinp1, Bmp8a, Mx1

TABLE 23
Genes with higher expression level in NV-ECs compared to V-ECs in
mesenteric lymph node (7 genes)
Gene name
Ifi206, Itih2, Klrb1f*, Elane, Gm49284, Pkhd1l1, Ahsg

TABLE 24
Genes with higher expression level in NV-ECs compared to V-ECs in
Peyer's patch (82 genes)
Gene name
Fasl*, Sh2d1b1, Cd244a*, Ifi208, Traf3ip3, Pnpla7, Rapgef4os3, Sla2,
Fam189b, Lingo4, Cd160, Lck, Fgr, Clnk, Txk, Selplg*, Pvrig*,
Trbj1-1, Gm28053, Gimap7, Gimap3, Il12rb2*, Gm6637, Klrb1a*,
Klrb1*, Klrb1c*, Klrb1b*, Klrd1*, Klra5*, Fbxo27, Nkg7*, Mrgpra9,
Itgad, 5830411N06Rik, Lsp1, Zmat4, Il12rb1*, Adcy7*, Tox3, Cd3g*,
Gm33699, Rasgrf1, Prf1*, 4933426K07Rik, Gm45605, Itgae, Ccl5,
Chad, Cd7* Gm36723, 5830428M24Rik, AU015791, Vipr2*, Tcrg-C1,
Tcorg-C4, A730091E23Rik, Gzma, Trdv4, Gzmb, Dock5, Gpr18*, Il7r*,
Apol7e, Tmprss6*, Il2rb*, Bex6, AC140186.1, Cd200r4, Cd200r2,
Cd96*, Baiap3, Lta, Dennd1c, AC102342.2, Tbc1d10c, Ctsw, Pim2,
Sash3, Stk26, Mtmr1, Zfp92, Cxcr3*

TABLE 25
Genes with higher expression level in NV-ECs compared to V-ECs in
thymus (319 genes)
Gene name
Slco5a1*, Tfap2b, Gm17767, Mstn, Aox4, Mreg, Wnt10a, Ccl20,
Gm29483, Crocc2, Gm29461, Serpinb12, Serpinb3a, Serpinb11,
Serpinb7, Serpinb2, Dpp10*, C4bp, Chil1, Lad1, Tnnt2, Pkp1, Rgs13,
Brinp3, Angptl1, Nectin4*, Ifi207, Ifi205, Marc1, Cacna1b*, Obp2a,
Stkld1, Dapl1, Myo3b, Cybrd1*, Smtnl1, 4833423E24Rik, Bbox1,
Rhov, Tgm5, Prom2*, A730036l17Rik, Oxt, Erv3, Prokr2*, Pax1,
Bpifa1, Bpifb9b, Cnbd2, Gdap1l1, Bmp7, Il7, Fabp9, Gpr87*, Il12a,
Sfrp2, Bglap3, Muc1*, S100a14, S100a3, Rptn, Hist2h4, Ankrd35,
Chil3, Chil4, Chil6, Fndc7, Calb1, Cfap206, Aqp3*, Slc46a2*, Dio1*,
Ttc39a, Slc5a9*, Skint8, Skint1, Skint4, Skint3, Skint9, Skint2*,
Skint10, Cd52, Ubxn11, Slc30a2*, Grhl3, Arhgef10l, Fhad1, Tnfrsf8*,
Car6, Ttll10, Gnat3, Drc1, Cpz, Psapl1, Sel1l3, Csn2, Gc, Bmp3,
Adgrd1*, Cldn4*, Ache, Ufsp1, Kpna7, Pip, Inmt, Tnip3, Tacstd2*,
Sh2d6, Asprv1, Il5ra*, Lrrc23, Gnb3, Sconn1a*, BC064078, Iapp,
Ceacam15, Mill1, Psg25, Pinlyp, Lypd3*, Cyp2b19, Cyp2g1, Cyp2a5,
Fcgbp, Syne4, Atp4a*, Sbsn, Dmkn, Ceacam18, Klk5, Klk1b26, Spib,
Fgf21, Fut1*, Abcc8, Saa3, Saa1, Apba2, Wnt11, Stk33, Calcb, Gp2,
Zp2*, Il27, Kndc1, Utf1, Adam8*, Urah, Syt8*, Ccl25, Anxa10,
Spock3, Gm42031, Ces5a, Ccl22, Cdh8*, Ces2c, Ctrb1, Pabpn1l,
Ccdc151, Npsr1*, Pou2f3, Trim29, Tmprss4, Fxyd2, Dscaml1*,
2310030G06Rik, 1810046K07Rik, Sh2d7, Cyp1a2, Cilp, Gsta2, Snap91,
Ripply2, Pcolce2, Zmynd10, Ltf, 4930520O04Rik, Ccr4*, Rgs17, Adgb,
Perp*, Vnn3, Vnn1*, Ccdc162, Tbata, Aire, Bpifc, Hal, Lum, Epyc,
Slc26a10*, Sdr9c7, Il23a, Pla2g3, Myl7, Gfpt2, Fat2*, Drc3, Dnah9,
Myh4, Alox12e, Vmo1, Tekt1, Serpinf1, Foxn1, Slc13a2os, Wfdc18,
Tex14, Lpo, Ttll6, Gsdma3, Krt15, Krt14, Krt16, Krt17, Gm12349,
Kcnh6*, Dnaic2, Grin2c*, Cbr2, Zfp750, Cdhr3*, Gm10165, Pax9,
Zc2hc1c, Slc24a4*, Serpina9, Serpina3j, Itgb8*, Calm4, Prss16, Dsp,
Elovl2, Diras2, Crhbp, Rnf180, Mcidas, Fezf2, Cadps, Gm48239,
Cdhr1*, Mat1a, Gm8113, Rnase1, Psmb11, Gjb2*, Gjb6*, Fam167a,
Tdh, Scara5*, Cnmd, Gm6999, Gm49542, Itgbl1, Fgf14, C7, Capsl,
Cdh9*, Has2*, Ly6d, D730001G18Rik, Gm8221, Cyp2d12, Krt84, Krt5,
Krt77, Krt76, Krt79, Krt78, Krt18, Srl, Tvp23a, Spag6l, Liph, Trp63,
Atp13a4*, Pigz, Pla1a, Tmprss2*, Unc93a*, Smoc2, Sbpl, Igfals, Msln,
Ccdc78, Pi16, Pglyrp2, H2-Oa, H2-DMb2*, H2-Eb2, BC051142,
Ly6g6c, H2-M2, Crip3, Gnmt, Sult1c2, Ebi3, Cd70*, Clip4, Dsc3*,
Dsg1a*, Nme5, Gm94, Spink5, Cdx1, Actn3, Cst6, Lrrn4cl, Pga5, Lipm,
Htr7*, Plekhs1, Slc9a7*, Nox1*, Serpina7, Pak3, Reps2, Asb11

TABLE 26
Genes with higher expression level in NV-ECs compared to V-ECs in
visceral adipose tissue (24 genes)
Gene name
Acvr1c*, Mapk8ip1, Gm43820, Clrn1*, Gm43221, Gm16559, Vmn2r-
ps20, Gm42989, Gm43018, Col28a1, Lep, Gprin3, Gm45722,
Gm45721, Abca8b*, 4930549C15Rik, Gm15902, 4930579J19Rik,
Abcd2, Ptchd4, 4430402l18Rik, A930028N01Rik, Scd1, Prrg3

TABLE 27
Genes with higher expression level in NV-ECs compared to V-ECs in
small intestine (22 genes)
Gene name
Spink4, Gm28874, Atad3aos, AI839979, Dmp1, Gm43604, Wdr95,
Gkn1, Gkn2, Gm49058, Tnnt1, Gm44829, Gm45667, Cldn18*, Cspg5*,
4932442E05Rik, Agr2, Gm48958, Mcpt4, Tpsab1, Tff2, Tff1

TABLE 28
Genes with higher expression level in NV-ECs compared to V-ECs in
colon (14 genes)
Gene name
Map9, Gm5979, Osgin1, Olfm2, Kcnj5, Smad6, Gm19385, Gm34425,
Gm39460, Gm47398, Nrn1, Stc1, Arc, Iglv2

TABLE 29
Genes with higher expression level in NV-ECs compared to V-ECs in
subcutaneous adipose tissue (8 genes)
Gene name
Rbp7, Gm5, Tmem86a, Dusp26, Evpl, Allc, Pacsin1, Tmem35a

TABLE 30
Genes with higher expression level in NV-ECs compared to V-ECs in
bone tissue (195 genes)
Gene name
Stradb, Tuba4a, Rgs18, E330020D12Rik, F5, Nuf2, Fcer1g*, Slamf1*,
Cd84*, Fcer1a*, Fmn2, Gm38158, Card9, Gfi1b, Ttll11, Lrrc55*,
Gm13716, Rapsn, Lrp4*, Knstrn, Knl1, Atp8b4*, Slpi, Aurka, Cass4,
Rbm38, Tubb1, Cpa3, Ccna2, P2ry12*, Cks1b, Tnfaip8l2, Gclm,
Dapp1, 4930519L02Rik, Cdc20, Mpl*, Cdca8, E2f2, Padi2, Draxin,
Poln, Crmp1, Ppbp, Pf4, Hpse, Tesc, Hcar2*, Ccdc92, Bri3bp, Tfr2*,
Alox5ap, 1810058l24Rik, Mkrn1, 2210010C04Rik, Nt5c3, Snca, Add2,
Prokr1*, Gp9*, Chst13, Tmem40, Alox5, Tuba8, Clec12a*, Clec1b*,
Gp6*, Lig1, Host*, Cebpa, Ccne1, Napsa, Csrp3, E2f8, Prc1, Lat*,
Kif22, Rgs10, Taldo1, Fgf3, Ckap2, Hmgb2, Ankle1, Cyp4f18, Mcm5,
Gypa*, Asf1b, Syce2, Mylk3, Lrrc29, Gins2, AB124611, Spc24, Nlrx1,
Nxpe1-ps, Cyp11a1, Cd276, Rbpms2, Ccnb2, Aqp9*, Mns1, Cmtm7,
Stx11*, Myb, Cdk1, Gnaz, R3hdm4, Mmp19, Osm, Fignl1, Plek,
Hmmr, Grm6*, Il4, Adora2b*, Aurkb, Alox12, Alox15, Gp1ba*,
Spns3, P2rx1*, Slc6a4*, Slfn14, Ccl3, Ccl4, Mpo, Mmd*, Lsm12,
Itga2b, Kif18b, Itgb3*, Cd300lb*, Unc13d, Ube2o, Birc5, Mfsd2b*,
Rrm2, Prkar2b, Adssl1, Hist1h4i, Hist1h2ac, Hist1h1c, C530050E15Rik,
F13a1, Cks2, Hk3, Mxd3, Rgs14, F2rl2*, Gm30411, Depdc1b, Ppif,
Rnase12, Ltb4r1*, Mcpt8, Ctsg, Arl11, Angpt1, Sntb1, Wisp1, Grap2,
Cenpm, Racgap1, Bin2, I730030J21 Rik, Nfe2, Sec14l5, Gp5*,
Cd200r1*, Cd200r3, Runx1, Slc22a3*, Ccnf, Prss34, Rab44, Mdga1,
Mpig6b*, Ly6g6f*, Treml2*, Treml1*, Tnfsf14*, Emilin2, Hrh4*,
Kif20a, Smim3, Rab27b, Cd226*, Npas4, Fermt3, Ms4a2*, Ms4a3*,
Gata1, 4930502E18Rik, F630028O10Rik, Btk

TABLE 31
Genes with higher expression level in NV-ECs compared to V-ECs in
uterus (47 genes)
Gene name
D430040D24Rik, Fam166a, Snhg7os, Cfap157, Hc, Chac1, Gm45354,
Abca4*, Frmd3, Gpr3*, Gm19590, Gm42830, Fbxl18, D430018E03Rik,
D730045B01Rik, Gpr37*, Tas2r143, Tas2r135, A430078l02Rik,
Gm43904, Gsg1*, Nanos2, Ppm1n, Selenov, Tex29, Gm3336, Gm16485,
Gm10655, Col6a4, Efna2, Emid1, Slc16a11*, Sgca*, 1700001P01Rik,
Cpsf41, Scin, Dbpht2, 6430562O15Rik, Rem2, Cdh24*, Gm29776,
Gm6610, CT010583.2, Gm17103, Gm8801, 5031415H12Rik, Cc2d2b

TABLE 32
Genes with higher expression level in NV-ECs compared to V-ECs
in brain parenchyma (64 genes)
Gene name
Dnah7c, Slc39a10*, Gm28151, Gm37393, Slc19a3*, Platr22,
Hmcn1, Gm20631, Gpr155, Ldlrad3*, Gm13920, Slc1a2*, Bcan,
Slc44a5*, Rad54b, Gm11832, Lrp8*, Lrp8os2, Mfsd2a*, Grm3*,
Sema3c*, Rgs12, Tbc1d14, Tecrl, Gm15626, Mmd2, Slc7a1*,
Gpr85*, Gm9946, Slco1c1*, Lmntd1, Rasgrp4, Vstm2b, Gm45552,
Abcc6*, Igf1r*, Scube2, Lyrm1, Gm45191, Ncan, Slc7a5*,
BC048644, Car5a, Scn3b*, Gm10030, Mobp, Spock2, Ddc, Aldoc,
Adam11*, Myadml2os, Notum, Ntsr2*, Gdap10, Lrrn3*, Wnk2,
Tfrc*, Tagln3, Tiam1, Mmp25*, Apcdd1*, Slc1a1*, Efhc2, Slc7a3*

TABLE 33
Genes with higher expression level in NV-ECs compared to V-
ECs in choroid plexus (21 genes)
Gene name
Gm5530, Oaz2-ps, Car8, Mfap2, Sez6l*, Cpxm2, Ces2e, Plscr2*,
Ctdspl, Gm39464, Cd24a, Tbxa2r*, Ramp3*, Olfr1396, Gm4804,
Plpp1*, Gm6212, Rhpn1, Etv5, Clic6*, Rps6ka2

TABLE 34
Genes with higher expression level in NV-ECs compared to
V-ECs in pia matter (13 genes)
Gene name
Slc4a3*, Adh7, Reck, Nos1, Bpgm, Gkn3, Alpk3, Htra1, Lcat,
Elovl4, 9930038B18Rik, Gfra2, Bmx

TABLE 35
Genes with higher expression level in NV-ECs compared to
V-ECs in dura matter (3 genes)
Gene name
Olfml2a, Arsj, Igf2

TABLE 36
Genes with higher expression level in V-ECs compared to NV-ECs
in peripheral lymph node, mesenteric lymph node, Peyer's patch,
thymus, visceral adipose tissue, small intestine, colon, subcutaneous
adipose tissue, bone tissue, uterus and dura matter (pan-venular,
tolerate 0, 2 genes): cell surface molecules only
Gene name
Ackr1, Spint2

TABLE 37
Genes with higher expression level in V-ECs compared to NV-ECs
in peripheral lymph node, mesenteric lymph node, Peyer's patch,
thymus, visceral adipose tissue, small intestine, colon, subcutaneous
adipose tissue, bone tissue, uterus and dura matter (pan-venular,
tolerate 1, 11 genes): cell surface molecules only
Gene name
Ackr1, Cadm3, Adgrg6, Pcdh7, Sirpa, Vcam1, Lepr, Spint2, Il1r1,
Icam1, Psen2

TABLE 38
Genes with higher expression level in V-ECs compared to NV-ECs in
peripheral lymph node, mesenteric lymph node, Peyer's patch, thymus,
visceral adipose tissue, small intestine, colon, subcutaneous adipose
tissue, bone tissue, uterus, brain parenchyma, choroid plexus, pia and dura
matter (pan-venular, tolerate 3, 15 genes): cell surface molecules only
Gene name
Ackr1, Sele, Selp, Cadm3, Adgrg6, Vcam1, Pcdh7, Myof, Thsd7a,
Spint2, Il1r1, Procr, Icam1, Il6st, Psen2

TABLE 39
Genes with higher expression level in V-ECs compared to NV-
ECs in peripheral lymph node (3 genes): cell surface molecules only
Gene name
Dclk1, Gabra2, Kcng3

TABLE 40
Genes with higher expression level in V-ECs compared to NV-ECs
in mesenteric lymph node (1 gene): cell surface molecules only
Gene name
Susd2

TABLE 41
Genes with higher expression level in V-ECs compared to
NV-ECs in Peyer's patch (23 genes): cell surface molecules only
Gene name
Trpa1, Astn1, Kcnh1, Kcnq2, Syt6, Clca1, Dpp6, Slc10a4,
Cd177, Gabrb3, Slco3a1, Chrna3, Stra6, Entpd3, Madcam1,
Adcy1, Fstl4, Pirt, Slc18a3, Adam28, Faim2, Slc35g1, Adgrg4

TABLE 42
Genes with higher expression level in V-ECs compared to
NV-ECs thymus (8 genes): cell surface molecules only
Gene name
Gpr1, Lrrn4, Fndc5, Tnfrsf9, Upk3b, Upk1b, Kcnj15, Vldlr

TABLE 43
Genes with higher expression level in V-ECs compared to NV-
ECs in visceral adipose tissue (3 genes): cell surface molecules only
Gene name
Kcnb1, Ret, Kirrel3

TABLE 44
Genes with higher expression level in V-ECs compared to NV-ECs
in small intestine (119 genes): cell surface molecules only
Gene name
Il1r2, Icos, Lct, Fcgr4, Slamf6, Igsf9, Entpd8, Slc27a4, Slc5a12,
Slc2a2, P2ry13, Lrat, Fcrl1, Fcgr1, Enpep, Clca4b, Car9, Stra6l,
Csf3r, Tlr12, Slc2a5, Slc2a7, Slc5a1, Slc34a2, Abcg3, Pilra, Flt3,
Slc13a1, Clec5a, Mgam, Gpnmb, Mpp6, C3ar1, Clec4a2, Clec2h,
Clec2e, Clec7a, Klra2, Pirb, Lair1, Lilra5, Ptprh, Cd22, Slc7a9,
Cd33, Anpep, Gm44974, Slc28a1, Vmn2r69, Tmc5, Slc5a11, Il21r,
Ano9, Cdhr5, Slc10a2, March1, Slc51b, Aph1c, Mst1r, Slc26a6,
Cx3cr1, Ccr5, Calhm6, Slc16a10, Ggt1, Slc5a4a, Trpm2, Slc39a5,
Erbb3, Gal3st1, Npc1l1, Havcr2, Cd68, Cxcl16, Tm4sf5, Ccr10,
Cd300a, Cd300c2, Otop3, Slc16a5, Gpr65, Amn, Gpr132, Gpr141,
D130043K22Rik, Cd83, Cdhr2, Slc6a19, Trac, Tnfsf11, Slc15a1,
Dcstamp, Dgat1, Slc38a1, Clcn2, Slc51a, Cd86, Slc22a1, Vmn2r102,
Tmem8, Btnl2, Treml4, Adgre4, Adgre1, Abcg5, Abcg8, Slc3a1,
Mep1b, Csf1r, Naaladl1, Cd5, Cd6, Abcc2, Csf2ra, Cybb, Tlr13,
P2ry10, Ace2, Tlr7

TABLE 45
Genes with higher expression level in V-ECs compared to NV-ECs
in colon (42 genes): cell surface molecules only
Gene name
Il1rl1, Cdh19, Gpr161, Pla2r1, Gpr176, Jph2, Cdh26, Agtr1b, Kcna2,
Ank2, Clca4a, Trabd2b, Tspan1, Ptafr, Adam22, Ereg, Parm1, Ntrk3,
Olfr78, Aqp8, Mrgprf, Ednra, Hhip, Trpc6, Naalad2, Ncam1, Calhm5,
Ggt5, Avpr1a, Itga7, Sstr2, Slc26a3, Sgcg, Gpc6, Sdc2, Ephb3, Aqp4,
Pdgfrb, Adra2a, Slc6a14, Gpc3, Nlgn3

TABLE 46
Genes with higher expression level in V-ECs compared to NV-ECs
in subcutaneous adipose tissue (5 genes): cell surface molecules only
Gene name
Mpz, Cldn19, Slc36a2, Kcns3, Ntrk2

TABLE 47
Genes with higher expression level in V-ECs compared to NV-ECs in
bone tissue (36 genes): cell surface molecules only
Gene name
Jph1, Fcgr2b, Mrc1, Sirpa, Cdh4, Gpr88, Slc26a7, Sema3a, Mgst1,
Kcnj8, Abcc9, C5ar2, C5ar1, Ifitm10, Gpm6a, Cdh11, Epor, Clmp,
Paqr9, Slc22a14, Stab2, Plxnc1, Flt4, Rtn4rl1, Sectm1a, Unc5a, Adcy2,
Vstm4, Ptger2, Cldn10, Cxadr, H2-Q10, Slc16a12, Gdpd2, Gpr143, Tlr8

TABLE 48
Genes with higher expression level in V-ECs compared to NV-ECs in
uterus (7 genes): cell surface molecules only
Gene name
Tlr4, P2rx2, Kcna6, Emp1, Izumo1, Insr, Pcdhb2

TABLE 49
Genes with higher expression level in V-ECs compared to NV-ECs in
brain parenchyma (1 gene): cell surface molecules only
Gene name
Ryr3

TABLE 50
Genes with higher expression level in V-ECs compared to NV-ECs in
pia matter (2 genes): cell surface molecules only
Gene name
Lrrtm1, Gm6614

TABLE 51
Genes with higher expression level in V-ECs compared to NV-ECs in
dura matter (1 gene): cell surface molecules only
Gene name
Il17ra

TABLE 52
Genes with higher expression level in V-ECs compared to NV-ECs in
peripheral lymph node, mesenteric lymph node, and Peyer's patch (HEV
specific): cell surface molecules only
Gene name
Stra6, Relt, Susd2, Plxnb1

TABLE 53
Genes with higher expression level in NV-ECs compared to V-ECs in
peripheral lymph node, mesenteric lymph node, Peyer's patch, thymus,
visceral adipose tissue, small intestine, colon, subcutaneous adipose tissue,
bone tissue, uterus and dura matter (pan-non-venular, tolerate 0, 1 gene):
cell surface molecules only
Gene name
Notch4

TABLE 54
Genes with higher expression level in NV-ECs compared to V-ECs in
peripheral lymph node, mesenteric lymph node, Peyer's patch, thymus,
visceral adipose tissue, small intestine, colon, subcutaneous adipose
tissue, bone tissue, uterus and dura matter (pan-non-venular, tolerate 1, 2
genes): cell surface molecules only
Gene name
Efnb2, Notch4

TABLE 55
Genes with higher expression level in NV-ECs compared to V-ECs in
peripheral lymph node, mesenteric lymph node, Peyer's patch, thymus,
visceral adipose tissue, small intestine, colon, subcutaneous adipose
tissue, bone tissue, uterus, brain parenchyma, choroid plexus, pia and
dura matter (pan-non-venular, tolerate 3, 4 genes): cell surface molecules
only
Gene name
Ednrb, Efnb2, Notch4, Dll4

TABLE 56
Genes with higher expression level in NV-ECs compared to V-ECs in
peripheral lymph node (14 genes): cell surface molecules only
Gene name
Ush2a, Olfr1013, Vmn1r76, Vmn1r80, Vmn1r81, Trpm1, Ros1, Gpr22,
Pcdh20, Cdh12, Trhr, Grin2a, Chrm1, Gpr174

TABLE 57
Genes with higher expression level in NV-ECs compared to V-ECs in
mesenteric lymph node (1 gene): cell surface molecules only
Gene name
Klrb1f

TABLE 58
Genes with higher expression level in NV-ECs compared to V-ECs in
Peyer's patch (24 genes): cell surface molecules only
Gene name
Fasl, Cd244a, Selplg, Pvrig, Il12rb2, Klrb1a, Klrb1, Klrb1c, Klrb1b,
Klrd1, Klra5, Nkg7, Il12rb1, Adcy7, Cd3g, Prf1, Cd7, Vipr2, Gpr18,
Il7r, Tmprss6, Il2rb, Cd96, Cxcr3

TABLE 59
Genes with higher expression level in NV-ECs compared to V-ECs in
thymus (56 genes): cell surface molecules only
Gene name
Slco5a1, Dpp10, Nectin4, Cacna1b, Cybrd1, Prom2, Prokr2, Gpr87,
Muc1, Aqp3, Slc46a2, Dio1, Slc5a9, Skint2, Slc30a2, Tnfrsf8, Adgrd1,
Cldn4, Tacstd2, Il5ra, Scnn1a, Lypd3, Atp4a, Fut1, Zp2, Adam8, Syt8,
Cdh8, Npsr1, Dscaml1, Ccr4, Perp, Vnn1, Slc26a10, Fat2, Kcnh6, Grin2c,
Cdhr3, Slc24a4, Itgb8, Cdhr1,Gjb2, Gjb6, Scara5, Cdh9, Has2, Atp13a4,
Tmprss2, Unc93a, H2-DMb2, Cd70, Dsc3, Dsg1a, Htr7, Slc9a7, Nox1

TABLE 60
Genes with higher expression level in NV-ECs compared to V-ECs in
visceral adipose tissue (3 genes): cell surface molecules only
Gene name
Acvr1c, Clrn1, Abca8b

TABLE 61
Genes with higher expression level in NV-ECs compared to V-ECs in
small intestine (2 genes): cell surface molecules only
Gene name
Cldn18, Cspg5

TABLE 62
Genes with higher expression level in NV-ECs compared to V-ECs
in colon (4 genes): cell surface molecules only
Gene name
Dll4, Epha2, Kcnj5, Tnfsf9

TABLE 63
Genes with higher expression level in NV-ECs compared to V-ECs
in subcutaneous adipose tissue (1 gene): cell surface molecules only
Gene name
Hrh2

TABLE 64
Genes with higher expression level in NV-ECs compared to V-ECs
in bone tissue (44 genes): cell surface molecules only
Gene name
Fcer1g, Slamf1, Cd84, Fcer1a, Lrrc55, Lrp4, Atp8b4, P2ry12, Mpl,
Hcar2, Tfr2, Prokr1, Gp9, Clec12a, Clec1b, Gp6, Hcst, Lat, Gypa,
Aqp9, Stx11, Grm6, Adora2b, Gp1ba, P2rx1, Slc6a4, Mmd, Itgb3,
Cd300lb, Mfsd2b, F2rl2, Ltb4r1, Gp5, Cd200r1, Slc22a3, Mpig6b,
Ly6g6f, Treml2, Treml1, Tnfsf14, Hrh4, Cd226, Ms4a2, Ms4a3

TABLE 65
Genes with higher expression level in NV-ECs compared to
V-ECs in uterus (7 genes): cell surface molecules only
Gene name
Abca4, Gpr3, Gpr37, Gsg1, Slc16a11, Sgca, Cdh24

TABLE 66
Genes with higher expression level in NV-ECs compared to
V-ECs in brain parenchyma (24 genes): cell surface molecules only
Gene name
Slc39a10, Slc19a3, Ldlrad3, Slc1a2, Slc44a5, Lrp8, Mfsd2a, Grm3,
Sema3c, Slc7a1, Gpr85, Slco1c1, Abcc6, Igf1r, Slc7a5, Scn3b,
Adam11, Ntsr2, Lrrn3, Tfrc, Mmp25, Apcdd1, Slc1a1, Slc7a3

TABLE 67
Genes with higher expression level in NV-ECs compared to
V-ECs in choroid plexus (6 genes): cell surface molecules only
Gene name
Sez6l, Plscr2, Tbxa2r, Ramp3, Plpp1, Clic6

TABLE 68
Genes with higher expression level in NV-ECs compared to
V-ECs in pia matter (1 gene): cell surface molecules only
Gene name
Slc4a3

### Example 2. Identification of Genes Differentially Expressed in ECs (V-ECs and NV-ECs) in Specific Tissues Compared to Other Tissues

Using technical and analysis tools similar to those used in Example 1, genes were identified that had higher expression level in ECs (V-ECs and NV-ECs) in one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter compared to other tissues. These genes are listed in Tables 69-121. Tables 69-82 list genes whose expression level in V-ECs and/or NV-ECs is higher (fold change ≥1.9) in one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter compared to other tissues. Tables 83-96 list the cell surface molecule genes respectively from Tables 69-82. Tables 97-110 list genes whose expression level in both V-ECs and NV-ECs is higher (fold change ≥1.9) in one of peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter compared to other tissues and fold change comparison is ≤1.5 between V-EC and NV-EC in the given tissue. Tables 111-121 list the cell surface molecule genes, respectively, from Tables 97, 99-100, 102-108, and 110.

TABLE 69
Genes with higher expression level in V-ECs and/or NV-ECs in peripheral lymph node
compared to other tissues (53 genes)
Gene name
Creg2, Gpc1, Marco*, Nckap5, Atp1b1*, Rprm, Ttn, B2m, Adam33, Dclk1*, Gm42608, Gm38244,
2010016l18Rik, Gm42510, Brinp1, Bmp8a, 6030400A10Rik, Gm43509, Gm42635, Gabra2*, Igfbp7,
Cfap73, Gm10382, Pnmal2, Ccnd1, Clec4g*, Csmd1, 8030455M16Rik, Gm48822, Atcay, Gm47472,
Gm47477, Fam19a2, Nefh, Abca13*, Gm16170, Gas7, Serpina1b, Tgfbi, F2r*, B230110C06Rik,
Cacna2d3, Gm49298, Gm34643, Gm26684, Ly6i, Slc38a4*, Glycam1, AC171205.1, Mx1, Meiob, Kcng3*,
Slc14a1*

TABLE 70
Genes with higher expression level in V-ECs and/or NV-ECs in
mesenteric lymph node compared to other tissues (13 genes)
Gene name
Gm3052, Itih2, Gm42533, Lrtm2, Klrb1f*, Tcerg1l, Susd2*, Elane,
Spic, Gm36283, Gm11427, Ahsg, Hs3st6

TABLE 71
Genes with higher expression level in V-ECs and/or NV-ECs in Peyer's patch compared
to other tissues (145 genes)
Gene name
Trpa1*, Cyp27a1, Scg2, Gbx2, D130058E05Rik, Astn1*, Pappa2, Fasl*, Sh2d1b1, Cd244a*, Ifi208,
Kcnh1*, Traf3ip3, Gcg, Rapgef4os3, Neurod1, Fjx1, Chgb, Snap25, Sla2, Snhg11, Stmn3, Tm4sf4,
Fam189b, Cd160, Reg4, Syt6*, Clca1*, Kif12, Pcsk9, Fgr, Dpysl5, Ppp2r2c, Clnk, Txk, Slc10a4*, Afp,
Mapk10, Selplg*, Pvrig*, Tac1, Gm28053, Gimap7, Gimap3, Osbpl3, Nap1l5, Atoh1, Il17re*, Gm6637,
Lpar5*, Klrb1a*, Klrb1c*, Klrb1b*, Klrd1*, Klra5*, Cd177*, Ceacam10, Nkg7*, Tph1, Mrgpra9, Gabrb3*,
Slco3a1*, Gm7580, Ap3b2, Zg16, Itgad, 5830411N06Rik, Sct, Muc2, Zmat4, Slc18a1, Il12rb1*, Hsh2d,
Gm5159, Cd3g*, Chrna3*, Celf6, Gm33699, Scg3, Rasgrf1, Entpd3*, Cck, Vip, 4930466K18Rik, Prf1*,
Madcam1*, Adcy1*, Pirt*, Zmynd15, Itgae, BC030499, Ccl5, Pyy, Cdk3-ps, Cd7*, 5830428M24Rik,
Gm6566, Chga, Gm30238, Vipr2*, Gng4, Tcrg-C1, Tcrg-C4, A730091E23Rik, F12, Pcsk1, Gm18990,
Rab3c, Gzma, Slc18a3*, Trdv4, Gzmb, Adam28*, Gpr18*, Apol7e, Tmprss6*, Il2rb*, C1qtnf6, Sult4a1,
Prph, Faim2*, Vwa5b2, Bex6, AC140186.1, Cd200r4, Cd200r2, Cd96*, Syngr3, Baiap3, Lta, Dennd1c,
AC102342.2, Tbc1d10c, Ctsw, Pim2, Pcsk1n, Syn1, Zcchc12, Sash3, Stk26, Adgrg4*, Zfp92, Cxcr3*,
Bex2, Zcchc18

TABLE 72
Genes with higher expression level in V-ECs and/or NV-ECs in thymus compared to
other tissues (360 genes)
Gene name
Tfap2b, Tmeff2, Gm17767, Mstn, Aox4, Gpr1*, Mreg, Wnt10a, Ccl20, Gm29483, Crocc2, Gm29461,
Serpinb12, Serpinb3a, Serpinb11, Serpinb7, Serpinb2, C4bp, Chil1, Lad1, Tnnt2, Pkp1, Rgs13, Brinp3,
Nectin4*, Ifi207, Ifi205, Marc1, Hsd11b1, Pfkfb3, Cacna1b*, Obp2a, Lhx3, Stkld1, Gsn, Dapl1, Myo3b,
Cybrd1*, Smtnl1, Prg2, 4833423E24Rik, Large2, Bbox1, Rhov, Tgm5, Prom2*, Pdyn, Oxt, Erv3, Prokr2*,
Lrrn4*, Pax1, Bpifa1, Bpifb9b, Gdap1l1, Bmp7, Fabp9, Gpr87*, Il12a, Fgg, Sfrp2, Bglap3, Muc1*,
S100a14, S100a3, Rptn, Tchh, Ankrd35, Chil3, Chil4, Chil6, Fndc7, Ntng1, Calb1, Cfap206, Aqp3*,
Svep1, Slc46a2*, Dio1*, Slc5a9*, Skint8, Skint1, Skint4, Skint3, Skint9, Skint2*, Skint10, Slc30a2*, Grhl3,
Fhad1, Tnfrsf8*, Car6, Gnat3, Drc1, Cpz, Htra3, Psapl1, Sod3, Sel1l3, Csn2, Gc, Bmp3, Gbp6, Hnf1a,
Adgrd1*, Cldn4*, Upk3b*, Ache, Kpna7, Pip, Inmt, Tacstd2*, Sh2d6, Asprv1, Il5ra*, Lrrc23, Gnb3,
Scnn1a*, Fgf23, Tspan11*, BC064078, Iapp, Lrmp*, Tm7sf3*, Ceacam15, Mill1, Psg25, Pinlyp, Lypd3*,
Cyp2b19, Cyp2g1, Cyp2a5, Cyp2f2, Fcgbp, 2200002D01Rik, Ppp1r14a, Syne4, Upk1a*, Atp4a*, Dmkn,
Ceacam18, Klk5, Klk1b26, Spib, Fgf21, Fut1*, Abcc8, Saa3, Saa1, Apba2, Saxo2, Wnt11, P2ry2*, Stk33,
BC051019, Calcb, Gp2, Zp2*, Il27, Kctd13, Bag3, Kndc1, Utf1, Adam8*, Urah, Syt8*, Anxa10, Spock3,
Gm42031, Ces5a, Ccl22, Cdh8*, Ces2c, Smpd3, Ctrb1, Kcng4*, Pabpn1l, Mmp3, Ccdc151, Npsr1*,
St3gal4, Trim29, Fxyd2, Dscaml1*, 2310030G06Rik, 1810046K07Rik, Sh2d7, Crabp1, Cyp1a2, Cilp,
Lipc, Gsta2, Prss35, Snap91, Ripply2, Pcolce2, Col6a5, Zmynd10, Ltf, Ccr4*, Clec3b, Cdcp1*, Adgb,
Perp*, Vnn3, Vnn1*, Tbata, Aire, Bpifc, Hal, Lum, Epyc, Nts, Slc26a10*, Sdr9c7, Il23a, Pla2g3, Myl7,
Upp1, Timd2*, Gfpt2, Fat2*, Dnah9, Myh4, Alox12e, Vmo1, Serpinf1, Foxn1, Slc13a2os, Wfdc18, Lpo,
Epx, Gm525, Ttll6, Gsdma3, Krt15, Krt14, Krt16, Krt17, Gm12349, Kcnh6*, Dnaic2, Grin2c*, Cbr2,
Zfp750, Osr1, Cdhr3*, Gm10165, Egln3, Pax9, Hif1a, Rab15, Ltbp2, Slc24a4*, Serpina9, Serpina3j,
Itgb8*, Calm4, Gpx6, Prss16, Dsp, Elovl2, Diras2, Spock1, Thbs4, Crhbp, Rnf180, Mcidas, Fezf2, Cadps,
Gm48239, Cdhr1*, Mat1a, Gm8113, Rnase1, Psmb11, Gjb2*, Gjb6*, Fam167a, Tdh, Scara5*, Cnmd,
Gm6999, Gm49542, Itgbl1, Fgf14, C7, Capsl, Cdh9*, Has2*, D730001G18Rik, Gm8221, Apol11b,
Cyp2d12, Krt84, Krt5, Krt77, Krt76, Krt79, Krt78, Krt18, Igfbp6, Srl, Liph, Trp63, Atp13a4*, Pigz, Upk1b*,
Kcnj15*, Tmprss2*, Fndc1, Unc93a*, Smoc2, Thbs2, Sbpl, Igfals, MsIn, Ccdc78, Pi16, Pglyrp2, H2-Oa,
H2-DMb2*, H2-Eb2, Ly6g6c, H2-M2, Crip3, Gnmt, Ebi3, Cd70*, C3, Clip4, Dsc3*, Dsg1a*, Gm94, Spink5,
Lvrn*, Cdx1, Actn3, Cst6, Lrrn4cl, Pga5, Vps37c, Vldlr*, Lipm, Ifit2, Ifit1, Htr7*, Ppp1r3c, Avpi1, Plekhs1,
Mid1ip1, Slc9a7*, Cnga2*, Nox1*, Serpina7, Prps1, Pak3, Capn6, Reps2, Asb11

TABLE 73
Genes with higher expression level in V-ECs and/or NV-ECs in visceral adipose tissue
compared to other tissues (67 genes)
Gene name
4930594C11Rik, Gm43213, Gm38309, Acvr1c*, D930015M05Rik, Gm14210, Ankef1, Gm16751, Them5,
Gm43221, Gm42892, Cxxc4, Gm16559, Mcoln3*, Gm42949, Gm26857, Gm37900, Gm24636, Vmn2r-
ps20, Gm16503, C230096K16Rik, Gm43747, Gm42603, Gm42992, Gm42993, Gm42995, Gm42989,
Gm43018, Gm43187, Gm43188, Eln, Gm18753, Col28a1, Gm42856, Lep, Gm37397, Ret*, Adamtsl3,
Gm45464, Gm45721, Gm38217, A530064N14Rik, Gm48743, Gm48751, Gm9930, Gm48755, Gm48757,
Samd5, A830082N09Rik, Wnt9b, 4930549C15Rik, Gm48617, Gm29007, Gm15902, Gm40663, Adarb2,
4930579J19Rik, Gm47118, Gm47798, Prr5, Abcd2, Masp1, Rnf39, Ptchd4, Fhod3, Scd1, Prrg3

TABLE 74
Genes with higher expression level in V-ECs and/or NV-ECs in small intestine compared
to other tissues (485 genes)
Gene name
Sntg1, Kcnb2*, Il17a, Col19a1, Neurl3, Il1r2*, Gm16894, Gm37347, Gm3940, Cd28*, Ctla4*, Icos*, Ikzf2,
Vil1, A530040E14Rik, Gpr55*, Alpi, Akp3, Rab17, Gm37053, Sctr*, Lct*, AA986860, Il10, Pm20d1, Lax1*,
Elf3, Gm37759, A130071D04Rik, Rgs1, Swt1, Sec16b, Fmo4, Fcrla, Fcgr4*, ItIn1, Ly9*, Slamf7*,
Slamf6*, Igsf9*, Mptx2, Mcm10, Celf2, St8sia6, Malrd1, Spag6, Myo3a, Entpd8*, Sapcd2, Dnm1, Cytip,
Olfr1258, Olfr1259, Spi1, Slc5a12*, Ppp1r14d, Ckmt1, Il1b, Hck, Ocstamp, Hnf4g, Chmp4c, Gm5150,
Slc2a2*, Gpr171*, P2ry13*, Plch1, Sis, Rbm46, Lrat*, Forl1*, Rab25, Pklr, Tuft1, Cgn, Ctss,
E330034L11Rik, Fcgr1*, Gm5547, Eps813, Gm43745, Gstm3, Dpyd, Fabp2, Gimd1, Bank1, Mttp, Adh6a,
Gbp2b, Clca4b*, Trp53inp1, Rragd, Spink4, Car9*, Aldh1b1, Stra6l*, Skint6, Faah, Zswim5, Guca2b,
Edn2, Csf3r*, Tlr12*, Tmem54, Gm28874, Crybg2, Runx3, C1qb, C1qc, C1qa, Pla2g2d, Rnf186,
Tmem82, Slc2a5*, Slc2a7*, Atad3aos, Cnpy1, Cgref1, Al839979, Slc5a1*, Ppargc1a, Slc34a2*,
Gm43721, Gm43627, 9130230L23Rik, Stap1, Ugt2a3, Sult1b1, Csn1s1, Jchain, Slc4a4*, Cxcl2, Sowahb,
Cds1, Dmp1, Sdsl, Rpl31-ps6, Abhd11os, Mixipl, Ccl24, Gm43604, Pilra*, Pilrb2, Cyp3a13, Nxpe5,
Adap1, Card11, Cyp3a25, Flt3*, Wdr95, Cftr*, Slc13a1*, Clec5a*, Mgam*, Mpp6*, Gm32479, Hpgds,
Gm30211, Igkc, Fabp1, Reg3b, Reg3a, Reg1, Dqx1, Gkn1, Gkn2, B130021K23Rik, C3ar1*, Clec4a1,
Clec4a2*, Clec4n, Clec2h*, BC035044, Clec2e*, Cd69*, Clec7a*, Klra17, Klra2*, Gm19434, Gm44013,
Sult6b2, Gm49058, Pirb*, Lair1*, Lilra5*, Tnnt1, Ptprh*, Igsf23, Nlrp9b, Cd79a*, Erich4, Cyp2b10,
Vmn1r185*, Lgals4, Ryr1*, Cd22*, Slc7a9*, Cd33*, Myh14, 0610005C13Rik, Sult2b1, Ush1c, Siglech,
Anpep*, Gm44974, Slc28a1*, Vmn2r69*, Vmn2r70*, E230029C05Rik, Gm44860, Gm45221, Gm45220,
Gm45176, Gm45177, Gm44676, Mogat2, Stard10, Gm45667, Gm8979, Gm1966, Wee1, Pde3b,
Gm44777, Gm45033, Tmc5*, Slc5a11*, Il21r*, Nlrp6, Ano9*, Rassf7, Cdhr5*, Muc6, Cd209b, Slc10a2*,
Defa30, Defa24, Atp7b*, Gm26909, Unc5d*, Dusp4, C130073E24Rik, BC030870, March1*, Lrrc25, Isx,
Ccdc7a, Acp5, Pdzd3, Treh, Apoa1, 2900052N01Rik, Bco2, Pou2af1, Dnaja4, Slc51b*, Pls1, Rbp2,
Cldn18*, 5830418P13Rik, Mst1r*, Slc26a6*, Cspg5*, Cx3cr1*, Ccr9*, Ccr5*, Zc3h12d, Arfgef3, Il22ra2,
Gm26740, Smlr1, Slc16a10*, Gm47856, Gm48065, Pkib, Pla2g12b, Hkdc1, Pbld2, Ggt1*, Derl3, Vpreb3,
Slc5a4a*, Trpm2*, Misp, Reep6, Dot1l, Creb313, Smim24, Glipr1*, Lyz2, Il22, 4932442E05Rik, Gm4473,
Myo1a, Rdh7, Slc39a5*, Erbb3*, Gal3st1*, Npc1l1*, Cobl, Wwc1, 4933415A04Rik, Fabp6, Havcr2*,
Timd4, Csf2, Gm12230, Gm26534, Tnfrsf13b*, Tmem238l, Mgl2, Cxcl16*, Tm4sf5*, Aspa, Evi2a,
Wfdc17, Gm21885, Cwc25, Arl5c, Ccr10*, Nags, Cd79b*, Cd300a*, Cd300c2*, Otop3*, Trim80, Slc16a5*,
Myo15b, Pycr1, Apob, Agr2, Akap5, Gpx2, Arg2, Acot4, Gpr65*, Kcnk13*, Ifi27l2b, Bcl11b, Amn*, Pld4,
Gpr132*, Igha, Ighg2b, Ighj3, Ighj1, Tubal3, Ryr2*, Gpr141*, Hist1h1d, D130043K22Rik*, Ly86, Txndc5,
Cd83*, Cdhr2*, Tifab, Gm48357, Slc28a3*, Fbp2, Fbp1, Slc6a19*, Gm8062, Iqgap2, Naip6, Cd180*,
Gzmk, Rarb, Il17rb*, Chdh, Pnp2, Tmem253, Trac*, Cideb, Mcpt-ps1, Mcpt4, Gata4, Nuggc, Adra1a*,
1700108F19Rik, Tnfsf11*, Slain1, Slc15a1*, Gdnf, Otulinl, Destamp*, Anxa13, BC025446, Eppk1, Dgat1*,
Gpt, Apol7a, Apol10a, Apol10c-ps, Lgals2, Baiap2l2, Cyp2d26, Slc38a1*, Mefv, Smim22, Tnfrsf17*, Iglc1,
Iglv1, Iglv3, Clcn2*, Bdh1, Slc51a*, Gm15657, AC129574.2, Cd86*, Eaf2, Nr1i2, Tigit*, Plcxd2, Slc22a1*
Tpsab1, Tmem8*, Mapk13, Fgd2, AC163635.1, Tff2, Tff1, Cyp4f40, Cyp4f14, Btnl2*, Btn15-ps, Btnl6,
Btnl7-ps, Aif1, H2-Q1, H2-Q2, Gm11131, H2-Bl, Gm10499, Gm8909, H2-T3, Trim15, Trim40, Pla2g7,
1700071M16Rik, Treml4*, Unc5cl, C230085N15Rik, 5830444F18Rik, Adgre4*, Adgre1*, Themis3,
Epb41l3, Nlrc4, Abcg5*, Abcg8*, Slc3a1*, Mep1b*, AC161438.1, Myo7b, Mzb1, Csf1r*, Onecut2, Myo5b,
Cndp1, 2010003K11Rik, Ovol1, Naaladl1*, Tkfc, Cd5*, Cd6*, Ms4a18, Ms4a12, Ms4a7, Ms4a4a,
Ms4a6c, BE692007, Mpeg1, Ifit1bl2, Cyp2c65, Cyp2c66, Abcc2*, Csf2ra*, Glod5, Cybb*, Otc, Cfp,
5430427019Rik, Tlr13*, P2ry10*, Gm6377, Tlr7*

TABLE 75
Genes with higher expression level in V-ECs and/or NV-ECs in colon compared to other
tissues (171 genes)
Gene name
Il1rl1*, Col3a1, E330011M16Rik, Des, Kcne4, Nmur1*, Cdh19*, Gm8818, Gpr161*, Olfml2b, Mptx1,
Grem2, Col5a1, Ncs1, Hmcn2, Tnfaip6, Pla2r1*, Klhl23, Chn1, Pde1a, Ypel4, Gm13889, Pamr1, Grem1,
Actc1, Gpr176*, Gm14010, Jph2*, Nfatc2, Car1, Agtr1b*, Ccdc39, Gm42899, C130075A20Rik, Postn,
Sptssb, Gucy1a1, Thbs3, Sprr2a3, Selenbp1, Hsd3b3, Hao2, Kcna2*, Ank2*, Clca4a*, St6galnac5, Tnc,
8030451A03Rik, Prkaa2, Podn, Tspan1*, Guca2a, Ptafr*, Nphp4, Cfap69, Ablim2, Fgfbp1, Ereg*,
Parm1*, Arhgap24, Dennd2a, Actg2, Pdzrn3, Gm36640, Zfp536, Klk14, Klk10, Plekha4, Sytl2, Olfr558*,
Olfr78*, Dkk3, Mical2, Acsm3, Aqp8*, Mrgprf*, Pdlim3, Ankrd37, Gm45407, Gm45714, Ednra*, Hhip*,
Tppp3, Osgin1, Crispld2, Trpc6*, Olfm2, Col5a3, Bmper, Konj5, Usp2, Nnmt, Ncam1*, Bmp5, Gm34829,
Gm22595, Gm34425, Gm39460, Tcf21, Lama2, Calhm5*, Ppil6, Gm47398, Fabp7, Ggt5*, Gm19688,
Col6a2, Col6a1, Fstl3, Ntn4, Mgat4c, Avpr1a*, Gli1, Mettl7b, Il5, 1810065E05Rik, Ccl11, Mapt, Sstr2*,
Slc16a3*, Alkal2, Slc26a3*, Dgkb, Akap6, Galnt16, Ttc9, Tgfb3, Meg3, Rtl1, Rian, Gm37899,
B830012L14Rik, Nrn1, Aspn, Pitx1, Tmem171, 2610528A11Rik, Sgcg*, C1qtnf3, Gm32618, Sdc2*,
Matn2, Colec10, Gsdmc2, Gsdmc3, Gsdmc4, Arc, 2010109l03Rik, Cyp2d34, Pla2g10, Myh11, Iglc3,
Iglv2, Retnlb, Abi3bp, Tnxb, Efhb, Tubb4a, Aqp4*, Garem1, Ppp2r2b, Gna14, Mamdc2, Cyp2c55, Cpn1,
Adra2a*, Hspa12a, Slc6a14*, Lonrf3, Gpc3*, Amelx

TABLE 76
Genes with higher expression level in V-ECs and/or NV-ECs in
subcutaneous adipose tissue compared to other tissues (29 genes)
Gene name
Mlph, Mpz*, Fabp5, Ugt8a, Mos, Hrct1, Cldnl9*, Rbp7, Cxcl9, Tbx5,
Tmem86a, Dusp26, Pth1r*, Pbld1, Slc36a2*, Unc119, Gm11542,
Evpl, Fasn, Allc, Hrh2*, Pacsin1, Angptl4, Cidea, Mbp,
Sfrp5, Slitrk4*, Tmem35a, Drp2

TABLE 77
Genes with higher expression level in V-ECs and/or NV-ECs in bone tissue compared to
other tissues (432 genes)
Gene name
Jph1*, Igfbp2, Tuba4a, Serpine2, Kif1a, Bok*, Tmem37, Mgat5, Gm16083, Cdk18, Ptpn7, Kif21b, Rgs18,
E330020D12Rik, Npl, Fcrlb, Fcgr2b*, Fcer1g*, Slamf1*, Cd84*, Fcer1a*, Gm38158, Lefty1, Susd4, Prkcq,
Mrc1*, Gfi1b, Fam102a, Ttll11, Cd302, Pdk1, Fkbp7, Tfpi, Lrrc55*, Gm13716, Rapsn, Gchfr, Capn3,
Sord, Dut, Fgf7, Atp8b4*, Sirpa*, Gfra4, Snx5, Ralgapa2, A930019D19Rik, Scand1, Gm826, Mafb, Sgk2,
Mybl2, Wisp2, Pabpc1l, Matn4, Ripor3, Cass4, Rbm38, Tubb1, Cdh4*, Bhlhe22, Cpa3, Gm43672,
Pabpc4l, Maml3, P2ry12*, Ptx3, Shox2, Lysmd1, Tnfaip812, Gpr88*, Npnt, Dapp1, Adh6b,
4930519L02Rik, Ak5, Al115009, Slc26a7*, Gm11837, Ttpa, Enho, Clta, Hemgn, Msantd3, Aldob, Susd1,
Snx30, Zfp618, 4933424M12Rik, Foxd2os, Mpl*, Zfp691, Hivep3, Marcksl1, Rhd*, Padi2, Gm13074,
Fblim1, Plod1, Draxin, Cenps, Fam213b, C1qtnf12, Sema3a*, Gm42676, Gm42675, Gm43719, Crygn,
Emilin1, Gm1673, Nat8l, Poln, Sh3bp2, Crmp1, A730089K16Rik, Gm43431, Ppbp, Pf4, Gm33370,
Gm8013, Antxr2*, Hpse, Ibsp, Spp1, Ccdc60, Tesc, Ccdc92, Bri3bp, Tfr2*, Snx8, Gm15411, Alox5ap,
1700028E10Rik, Tfpi2, Gng11, Tfec, Akr1b8, Mkrn1, 2210010C04Rik, Kel*, Snca, Lbx2, Add2,
Gm28719, Prokr1*, Gp9*, Chst13, Tmem40, Zfand4, Alox5, Tuba8, Cracr2a, Clec12a*, Clec1b*, Hebp1,
Mgst1*, Kcnj8*, Abcc9*, Gp6*, C5ar2*, C5ar1*, Ap2s1, Calm3, Gmfg, Hcst*, Gm38340, Rcn3, Csrp3,
E2f8, Gm2788, Kif7, Pex11a, Myo7a, Lat*, Rgs10, Adam12*, Gm19514, Fgf3, Ank1, Gm5345, Wdr17,
Gpm6a*, Gm45264, Hmgb2, Homer3, Lsm4, Tmem221, Cyp4f18, Mcm5, Gypa*, Asf1b, Syce2, Klf1,
Mylk3, 4921524J17Rik, Adgrg3*, Cdh11*, Cmtm3, Hp, Gm16118, Maf, Gins2, Agt, AB124611, Epor*,
Gm37192, Slc37a2, Oaf, Nlrx1, Nxpe1-ps, Nrg4*, Scamp5*, Cyp11a1, Cd276, Dapk2, Aqp9*, Mns1,
Hmgcll1, Slc9a9*, Paqr9*, Trf, Cmtm7, D330037F02Rik, Slc22a14*, Stx11*, Vta1, Dse, Mett124, Oit3,
Gnaz, Sumo3, Lrrc3, Gm16268, Stab2*, Gm16280, Gm16270, Gm16271, Nav3, Wif1, Srgap1, Gm48804,
Gpr182*, Rdh16, Osbp2, Tcn2, Osm, Ap1b1, Gas2l1, Fignl1, Rnf145, Flt4*, Ltc4s, Grm6*, Kif3a, Il4,
Gpx3, Ccdc69, Aldh3a1*, Adora2b*, Mmgt2*, Alox15, Arrb2, Gp1ba*, Spns3, P2rx1*, Trpv3*, Rtn4rl1*,
Slc43a2*, Slc6a4*, Abhd15, Dhrs13, Tmem132e, Slfn14, Ccl3, Ccl4, Mpo, Mmd*, Igfbp4, Tmem106a,
Lsm12, Slc4a1*, Itga2b, Kif18b, Itgb3*, Limd2, Gm11707, Gprc5c*, Cd300lb*, Unc13d, Birc5, Engase,
Rbfox3, Sectm1a*, Mfsd2b*, Rrm2, Prkar2b, Ispd, Nrcam*, Snx6, L2hgdh, Cdkl1, 4930512B01Rik,
Map4k5, Ston2, Gm8378, Lgmn, Serpina3c, Serpina3i, Adssl1, Epdr1, Sfrp4, Hist1h4i, Hist1h2ac, F13a1,
Mylip, Gmpr, Sfxn1, Unc5a*, Hk3, Mxd3, Dbn1, Dok3, Cxcl14, Adcy2*, D030007L05Rik, Irx2, Gm32067,
F2rl2*, Naip2, Gm30411, Ipo11, Fst, Dnase1l3, Ppif, Nt5dc2, Stab1*, Tmem273, Fam170b, Vstm4*,
Ptger2*, Rnase10, Rnase12, Rnase4, Mmp14*, Slc7a8*, Mcpt8, Ctsg, Fam124a, Loxl2, Ccdc122,
Dnajc15, Selenop, Gm10384, Lrp12*, Rspo2, Wisp1, Pdzrn4, Lalba, Tfcp2, Bin2, I730030J21Rik, Krt87,
Nfe2, Sec14l5, Gm41414, Rbfox1, Kng2, Kng1, Adipoq, Gm6630, P3h2, Gp5*, Cd200r1*, Cd200r3,
Zbtb11os1, 9430053O09Rik, Gm37606, E330011O21Rik, Cxadr*, Adamts5, Runx1, Slc22a3*, Prss34,
Rab11fip3, Rab44, Mdga1, Mpig6b*, Ly6g6d, Ly6g6e, Ly6g6f*, H2-Q10*, Trim10, Mdfi, Treml2*, Treml1*,
Sh3gl1, Tnfsf14*, Emilin2, Hrh4*, Bin1, Prob1, Snx2, Smim3, Rab27b, Ska1, Lipg, Rbfa, Cd226*, Rhod,
Sptbn2, Npas4, Sipa1, Fermt3, Incenp, Ms4a2*, Ms4a3*, Dock8, Glis3, Conj, Gata1, Ap3s1-ps1,
4930502E18Rik, F8a, F630028O10Rik, Gdpd2*, Mum1l1, Morc4, Tmem164, Il13ra2, Gpr143*, Sh3kbp1,
Tlr8*

TABLE 78
Genes with higher expression level in V-ECs and/or NV-ECs in uterus compared to other
tissues (235 genes)
Gene name
Gm37265, Aox3, Eef1b2, Map2, Gm10558, Pid1, 2610012C04Rik, Gm4793, Gm38399, Gm7694, Pax8,
Fam166a, Snhg7os, Olfm1, Hc, Hoxd11, Hoxd10, Meis2, Chac1, Sptbn5, Dusp2, Astl, Adra2b*, Dnmt3b,
Wfdc2, C030034L19Rik, Gm43414, Jade1, Rxfp1*, Gm26984, Pdgfc, Mex3a, 4731419I09Rik, Gm23723,
Gm45354, Gm17690, Olfml3, 5330417C22Rik*, Cdc14a, F3*, Dkk2, Atp6v0d2, Olfr270, Tnfsf8*, Tlr4*,
Frmd3, Bnc2, Adamtsl1, Lepr*, Gm26615, Gpr3*, Zfp593, Sh2d5, Kif17, Gm13053, Tmem51os1, Prkcz,
Gm26825, Atg9b, Gm43852, Gm19590, P2rx2*, Sgsm1, Ccdc63, Rad9b, Gm42830, Tmem132c,
5930412G12Rik, Fbxl18, D430018E03Rik, D730045B01Rik, Cttnbp2, Gpr37*, Flnc, Tas2r143, Tas2r135,
4921507P07Rik, Hoxa11, Gm44389, Rpia, Elmod3, Retsat, Gm45053, Gm26640, Fbxo41, Gm43917,
A430078I02Rik, Gm43904, Gm44423, Gm15737, Ttll3, Gm6375, Gsg1*, Emp1*, Rerg, Bcat1, Gm47692,
Rnf225, Nanos2, Selenov, AF357399, Izumo1*, Mamstr, Spon1, Prap1, Gm18210, Insr*, Vmn2r-ps88,
Gm16180, Arhgef7, Tex29, Nek5, Zfp703, Actg-ps1, AV026068, Gm3336, Pde4c, 6330537M06Rik, Sall1,
Hsd11b2, Cdh3*, Gm20416, Pgr, Gm16485, Gm36198, Gm16853, Gm27162, Gm8543, Gramd1b*,
3110039I08Rik, Gm48742, Gm48740, 1600029O15Rik, Gm10655, Ankdd1a, Gm5366, Cd109,
Gm39384, Col6a4, 4930553I21Rik, Gm26359, Gm10941, Efna2, Emid1, Eif4a1, Slc16a11*, Tbx4,
Vmp1*, Ypel2, Sgca*, Gm53, Hoxb9, 1700001P01Rik, Hspb9, Stat5a, Scn4a*, Cacng4*, Dnah17, Nptx1,
Actg1, Gm6682, Greb1, Scin, Dbpht2, Clmn, Ror2*, 1700014D04Rik, Gm47586, Gm47603, Gm47585,
Gm47602, Gm16907, Gm16133, 1700023H06Rik, 6430562O15Rik, Thrb, Gm48321, Gm49308, Rem2,
Rec8, Trim13, Gm7232, Gm8174, Osr2, Gm4740, Gm26704, Samd12, 1700015H07Rik, Gm27782,
Gm6610, Gm49410, Apol8, Foxred2, Mpst, Gm20621, Mapk8ip2, Acr, A130051J06Rik, Gm49173,
Aqp5*, 6030408B16Rik, CT010583.2, Fgd4, Gm17103, AC163694.1, Chodl, Adamts1, Bach1,
AC134560.1, Gm7831, Prss28, Prss29, Cacna1h*, AC127341.3, Gm8801, Plin5, Sema6b*,
5031415H12Rik, Gm20703, Pcdhb2*, BC037039, Pcdhgc4*, Gm29994, A330084C13Rik, Clcf1, Neat1,
Gm42067, Fads2*, Cc2d2b, Gsto2, Ddx3x, Agtr2*, Gm5388, Tpt1-ps6, Xist

TABLE 79
Genes with higher expression level in V-ECs and/or NV-ECs in brain parenchyma
compared to other tissues (76 genes)
Gene name
Slc39a10*, Gm37393, Slc19a3*, Platr22, Gm13324, Gm13571, Gpr155, Ccdc141, Slc1a2*, Gatm, Bcan,
Ciart, Gm11827, Rad54b, Gm11832, Coro2a, Tmeff1*, Ak4, Lrp8*, Lrp8os2, Lao1, Mfsd2a*, Megf6,
Grm3*, Tcf23, Tbc1d14, D630030B08Rik, Gm42466, Tecrl, Gm15626, Mmd2, Gpr85*, 4930517G19Rik,
Gm45828, Gm44981, Gm9946, Slc6a13*, Slco1c1*, Lmntd1, Hif3a, Vstm2b, Atp10a*, Igf1r*, Tmc7*,
Lyrm1, Dnah3, Gm45191, Ncan, Gm32352, Slc7a5*, BC048644, Car5a, Gm10030, Mobp, Spock2,
Gm48495, Gm9176, Pik3ip1*, Ddc, Aldoc, Adam11*, Gm11766, Myadml2os, Notum, Notumos, Ntsr2*,
Lrrn3*, Fam107a, Tfrc*, Tagln3, Tiam1, Camk2a, Slc1a1*, Sgms1*, Efhc2, Slc7a3*

TABLE 80
Genes with higher expression level in V-ECs and/or NV-ECs in choroid plexus compared
to other tissues (78 genes)
Gene name
1500015010Rik, Gm5530, Kcnj13, Ralgps2, Brinp2, Slc39a12*, Slc4a10*, Col9a3, Serpini1, Zfp697,
Plppr4*, Oaz2-ps, Car8, Gm12892, Col8a2, Mfap2, Steap1*, Steap4*, Sez6l*, Slc29a4*, Kl*, Dync1i1,
Iqub, Grm8*, Slc13a4*, 4933431G14Rik, Tmem72, Exoc3l2, Lrp3, Gm44951, Folr1, Cpxm2, Gm44891,
Slc5a5*, Mt3, Drc7, Ces2e, Vat1l, Tmem136, Rdx, Fam81a, Plscr2*, Gm2415, Ctdspl, Gm39464, Moxd1,
Cd24a, Gm4798, Tbxa2r*, Ptprq, Ramp3*, Nsg2*, Olfr1396, Car10, Gm4804, Sostdc1, Plpp1*, Otx2,
2900040C04Rik, Pcdh9*, Gm17922, Gm6212, Gm7543, Rhpn1, Emp2*, Etv5, Lsamp, Kcne2*, Clic6*,
Rps6ka2, Nrxn1*, Ttr, Mapk4, Prr32, Atp2b3*, Stard8, Htr2c*, Frmpd4

TABLE 81
Genes with higher expression level in V-ECs and/or NV-ECs
in pia matter compared to other tissues (18 genes)
Gene name
Slc4a3*, Irf6, Adh7, Reck, Nos1, Lrrtm1*, Gkn3, Alpk3, 5830468F06Rik,
Elovl4, 9930038B18Rik, Sntg2, Gfra2, 1810041H14Rik,
Tbx1, 2810407A14Rik, Lama1, Bmx

TABLE 82
Genes with higher expression level in V-ECs and/or NV-ECs
in dura matter compared to other tissues (20 genes)
Gene name
Eya1, Kcnt2*, Prg4, Gm30694, Gm37422, Kcnt1*, Arsj, Gm11210,
Gm5552, Gm22816, Prr15, Wipf3, Gm25535, Il17ra*, Olr1*,
Pthlh, Gm26802, Nrxn3*, Gm17083, AC151299.1

TABLE 83
Genes with higher expression level in V-ECs and/or NV-ECs
in peripheral lymph node compared to other tissues (10 genes):
cell surface molecules only
Gene name
Marco, Atp1b1, Dclk1, Gabra2, Clec4g, Abca13,
F2r, Slc38a4, Kong3, Slc14a1

TABLE 84
Genes with higher expression level in V-ECs and/or NV-ECs
in mesenteric lymph node compared to other tissues
(2 genes): cell surface molecules only
Gene name
Klrb1f, Susd2

TABLE 85
Genes with higher expression level in V-ECs and/or NV-ECs
in Peyer's patch compared to other tissues
(40 genes): cell surface molecules only
Gene name
Trpa1, Astn1, Pappa2, Fasl, Cd244a, Kcnh1, Syt6, Clca1, Slc10a4, Selplg,
Pvrig, Il17re, Lpar5, Klrb1a, Klrb1c, Klrb1b, Klrd1, Klra5, Cd177, Nkg7,
Gabrb3, Slco3a1, Il12rb1, Cd3g, Chrna3Entpd3, Prf1, Madcam1, Adcy1,
Pirt, Cd7, Vipr2, Slc18a3, Adam28, Gpr18, Tmprss6, Il2rb, Faim2, Cd96,
Adgrg4, Cxcr3

TABLE 86
Genes with higher expression level in V-ECs and/or NV-ECs in thymus compared to
other tissues (70 genes): cell surface molecules only
Gene name
Gpr1, Nectin4, Cacna1b, Cybrd1, Prom2, Prokr2, Lrrn4, Gpr87, Muc1, Aqp3, Slc46a2, Dio1, Slc5a9,
Skint2, Slc30a2, Tnfrsf8, Adgrd1, Cldn4, Upk3b, Tacstd2, Il5ra, Sonn1a, Tspan11, Lrmp, Tm7sf3, Lypd3,
Upk1a, Atp4a, Fut1, P2ry2, Zp2, Adam8, Syt8, Cdh8, Kcng4, Npsr1, Dscaml1, Ccr4, Cdcp1, Perp, Vnn1,
Slc26a10, Timd2, Fat2, Kcnh6, Grin2c, Cdhr3, Slc24a4, Itgb8, Cdhr1, Gjb2, Gjb6, Scara5, Cdh9, Has2, ,
Atp13a4, Upk1b, Kcnj15, Tmprss2, Unc93a, H2-DMb2, Cd70, Dsc3, Dsg1a, Lvrn, Vldlr, Htr7, Slc9a7,
Cnga2, Nox1

TABLE 87
Genes with higher expression level in V-ECs and/or NV-ECs
in visceral adipose tissue compared to other tissues
(3 genes): cell surface molecules only
Gene name
Acvr1c, Mcoln3, Ret

TABLE 88
Genes with higher expression level in V-ECs and/or NV-ECs in small intestine compared
to other tissues (132 genes): cell surface molecules only
Gene name
Sntg1, Kcnb2, Il1r2, Cd28, Ctla4, Icos, Gpr55, Sctr, Lct, Lax1, Fcgr4, Ly9, Slamf7, Slamf6, Igsf9, Entpd8,
Slc5a12, , Slc2a2, Gpr171, P2ry13, Lrat, Fcrl1, Fcgr1, Clca4b, Car9, Stra6l, Csf3r, Tlr12, Slc2a5, Slc2a7,
Slc5a1, Slc34a2, Slc4a4, Pilra, Flt3, Cftr, Slc13a1, Clec5a, Mgam, Mpp6, C3ar1, Clec4a2, Clec2h,
Clec2e, Cd69, Clec7a, Klra2, Pirb, Lair1, Lilra5, Ptprh, Cd79a, Vmn1r185, Ryr1, Cd22, Slc7a9, Cd33,
Anpep, Slc28a1, Vmn2r69, Vmn2r70, Tmc5, Slc5a11, Il21r, Ano9, Cdhr5, Slc10a2, Atp7b, Unc5d,
March1, Slc51b, Cldn18, Mst1r, Slc26a6, Cspg5, Cx3cr1, Ccr9, Ccr5, Slc16a10, Ggt1, Slc5a4a, Trpm2,
Glipr1, Slc39a5, Erbb3, Gal3st1, Npc1l1, Havcr2, Tnfrsf13b, Cxcl16, Tm4sf5, Ccr10, Cd79b, Cd300a,
Cd300c2, Otop3, Slc16a5, Gpr65, Kcnk13, Amn, Gpr132, Ryr2, Gpr141, D130043K22Rik, Cd83, Cdhr2,
Slc28a3, Slc6a19, Cd180, Il17rb, Trac, Adra1a, Tnfsf11, Slc15a1, Dcstamp, Dgat1, Slc38a1, Tnfrsf17,
Clcn2, Slc51a, Cd86, Tigit, Slc22a1, Tmem8, Btnl2, Treml4, Adgre4, Adgre1, Abcg5, Abcg8, Slc3a1,
Mep1b, Csf1r, Naaladl1, Cd5, Cd6, Abcc2, Csf2ra, Cybb, Tlr13, P2ry10, Tlr7

TABLE 89
Genes with higher expression level in V-ECs and/or NV-ECs in colon
compared to other tissues (35 genes): cell surface molecules only
Gene name
Il1rl1, Nmur1, Cdh19, Gpr161, Pla2r1, Gpr176, Jph2, Agtr1b, Kcna2,
Ank2, Clca4a, Tspan1, Ptafr, Ereg, Parm1, Olfr558, Olfr78, Aqp8,
Mrgprf, Ednra, Hhip, Trpc6, Ncam1, Calhm5, Ggt5, Avpr1a, Sstr2,
Slc16a3, Slc26a3, Sgcg, Sdc2, Aqp4, Adra2a, Slc6a14, Gpc3

TABLE 90
Genes with higher expression level in V-ECs and/or NV-ECs
in subcutaneous adipose tissue compared to other tissues
(6 genes): cell surface molecules only
Gene name
Mpz, Cldn19, Pth1r, Slc36a2, Hrh2, Slitrk4

TABLE 91
Genes with higher expression level in V-ECs and/or NV-ECs in bone tissue compared to
other tissues (94 genes): cell surface molecules only
Gene name
Jph1, Bok, Fcgr2b, Fcer1g, Slamf1, Cd84, Fcer1a, Mrc1, Lrrc55, Atp8b4, Sirpa, Cdh4, P2ry12, Gpr88,
Slc26a7, Mpl, Rhd, Sema3a, Antxr2, Tfr2, Kel, Prokr1, Gp9, Clec12a, Clec1b, Mgst1, Kcnj8, Abcc9, Gp6,
C5ar2, C5ar1, Hcst, Lat, Adam12, Gpm6a, Gypa, Adgrg3, Cdh11, Epor, Nrg4, Scamp5, Aqp9, Slc9a9,
Paqr9, Slc22a14, Stx11, Stab2, Gpr182, Flt4, Grm6, Aldh3a1, Adora2b, Mmgt2, Gp1ba, P2rx1, Trpv3,
Rtn4rl1, Slc43a2, Slc6a4, Mmd, Slc4a1, Itgb3, Gprc5c, Cd300lb, Sectm1a, Mfsd2b, Nrcam, Unc5a,
Adcy2, F2rl2, Stab1, Vstm4, Ptger2, Mmp14, Slc7a8, Lrp12, , Gp5, Cd200r1, Cxadr, Slc22a3, Mpig6b,
Ly6g6f, H2-Q10, Treml2, Treml1, Tnfsf14, Hrh4, Cd226, Ms4a2, Ms4a3, Gdpd2, Gpr143, Tlr8

TABLE 92
Genes with higher expression level in V-ECs and/or NV-ECs in uterus
compared to other tissues (30 genes): cell surface molecules only
Gene name
Adra2b, Rxfp1, 5330417C22Rik, F3, Tnfsf8, Tlr4, Lepr, Gpr3, , P2rx2,
Gpr37, Gsg1, Emp1, Izumo1, Insr, Cdh3, Gramd1b, , Slc16a11, Vmp1,
Sgca, Scn4a, Cacng4, Ror2, Aqp5, Cacna1h, Sema6b, Pcdhb2,
Pcdhgc4, Fads2, Agtr2

TABLE 93
Genes with higher expression level in V-ECs and/or NV-ECs in brain
parenchyma compared to other tissues (22 genes): cell surface molecules
only
Gene name
Slc39a10, Slc19a3, Slc1a2, Tmeff1, Lrp8, Mfsd2a, Grm3, Gpr85,
Slc6a13, Slco1c1, Atp10a, Igf1r, Tmc7, Slc7a5, Pik3ip1, Adam11,
Ntsr2, Lrrn3, Tfrc, Slc1a1, Sgms1, Slc7a3

TABLE 94
Genes with higher expression level in V-ECs and/or NV-ECs in choroid
plexus compared to other tissues (23 genes): cell surface molecules only
Gene name
Slc39a12, Slc4a10, Plppr4, Steap1, Steap4, Sez6l, Slc29a4, Kl, Grm8,
Slc13a4, Slc5a5, Plscr2, Tbxa2r, Ramp3, Nsg2, Plpp1, Pcdh9, Emp2,
Kcne2, Clic6, Nrxn1, Atp2b3, Htr2c

TABLE 95
Genes with higher expression level in V-ECs and/or NV-ECs in pia
matter compared to other tissues (2 genes): cell surface molecules only
Gene name
Slc4a3, Lrrtm1

TABLE 96
Genes with higher expression level in V-ECs and/or NV-ECs in dura
matter compared to other tissues (5 genes): cell surface molecules only
Gene name
Kcnt2, Kcnt1, Il17ra, Olr1, Nrxn3

TABLE 97
Genes with higher expression level in both V-ECs and NV-ECs in
peripheral lymph node compared to other tissues (10 genes)
Gene name
Marco*, Gm43509, Igfbp7, Pnmal2, Abca13*, F2r*, B230110C06Rik,
Gm49298, Meiob, Slc14a1*

TABLE 98
Genes with higher expression level in both V-ECs and NV-ECs in
mesenteric lymph node compared to other tissues (1 gene)
Gene name
Gm36283

TABLE 99
Genes with higher expression level in both V-ECs and NV-ECs in
Peyer's patch compared to other tissues (8 genes)
Gene name
Stmn3, Fgr, Zmynd15, Gm6566, Gm18990, C1qtnf6, Ctsw, Zcchc18

TABLE 100
Genes with higher expression level in both V-ECs and NV-ECs in
thymus compared to other tissues (17 genes)
Gene name
Lhx3, Large2, Lrmp*, Klk5, P2ry2*, Kctd13, Smpd3, Ctrb1, Kcng4*,
Lipc, Prss35, Col6a5, Thbs2, Lvrn*, Ifit2, Ifit1, Prps1

TABLE 101
Genes with higher expression level in both V-ECs and NV-ECs
in visceral adipose tissue compared to other tissues (26 genes)
Gene name
4930594C11Rik, Gm43213, Gm38309, D930015M05Rik, Gm14210,
Ankef1, Gm42892, Cxxc4, Gm37900, Gm24636, C230096K16Rik,
Gm43747, Gm42603, Gm42992, Gm42993, Gm42995, Gm43188,
Gm18753, Gm42856, Gm45464, A530064N14Rik, A830082N09Rik,
Gm48617, Gm47798, Prr5, Fhod3

TABLE 102
Genes with higher expression level in both V-ECs and NV-ECs
in small intestine compared to other tissues (76 genes)
Gene name
Sntg1, Col19a1, A530040E14Rik, Gm37053, Sctr*, Lax1*, Gm37759,
Swt1, Mcm10, Spag6, Plch1, Rab25, E330034L11Rik, Trp53inp1,
Spink4,Zswim5, Sult1b1, Jchain, Dmp1, Rpl31-ps6, Cftr*, Gm30211,
Igkc, Gm44013, Cyp2b10, Vmn1r185*, Slc7a9*, Wee1, Gm45033,
Nlrp6, Muc6, Gm26909, Unc5d*, Ccdc7a, Pou2af1, Arfgef3, Gm47856,
Pla2g12b, Hkdc1, Derl3, Vpreb3, Reep6, Dot1l, Glipr1*, Gm12230,
Tnfrsf13b*, Tm4sf5*, Arl5c, Pycr1, Akap5, Igha, Ighg2b, Ighj1,
Hist1h1d, Txndc5, Gm48357, Fbp1, Cd180*, Gzmk, Rarb, Il17rb*,
Tmem253, Nuggc, Adra1a*, Tnfrsf17*, Iglv3, Gm15657, AC129574.2,
Eaf2, Tigit*, Tff2, Gm11131, Slc3a1*, Myo7b, Mzb1, Onecut2

TABLE 103
Genes with higher expression level in both V-ECs and NV-ECs in
colon compared to other tissues (14 genes)
Gene name
Il1rl1*, E330011M16Rik, Nmur1*, Gm8818, Mptx1, Gm14010, Nfatc2,
Nphp4, Olfr558*, Bmper, Ppil6, Mapt, Ttc9, Lonrf3

TABLE 104
Genes with higher expression level in both V-ECs and NV-ECs in
subcutaneous adipose tissue compared to other tissues (6 genes)
Gene name
Fabp5, Tbx5, Pth1r*, Pbld1, Fasn, Angptl4

TABLE 105
Genes with higher expression level in both V-ECs and NV-ECs in bone
tissue compared to other tissues (66 genes)
Gene name
Igfbp2, Mgat5, Kif21b, Susd4, Prkcq, Fkbp7, Capn3, Sord,
A930019D19Rik, Mybl2, Ripor3, Gm43672, Maml3, Lysmd1, Npnt,
Enho, Clta, Msantd3, Susd1, Zfp618, Rhd*, Plod1, Emilin1, Sh3bp2,
Antxr2*, Tfpi2, Gng11, Lbx2, Zfand4, Hebp1, Calm3, Gm38340,
Adam12*, Gm19514, Homer3, Lsm4, Cmtm3, D330037F02Rik,
Gm48804, Osbp2, Gas211, Mmgt2*, Arrb2, Trpv3*, Limd2, Gm11707,
Rbfox3, Sfxn1, Ipo11, Nt5dc2, Mmp14*, Slc7a8*, Gm10384, Lalba,
SRbfox1, Gm37606, Ly6g6d, Mdfi, Sh3gl1, Bin1, Rbfa, ptbn2, Sipa1,
Dock8, Ap3s1-ps1, Sh3kbp1

TABLE 106
Genes with higher expression level in both V-ECs and NV-ECs in
uterus compared to other tissues (103 genes)
Gene name
Aox3, Map2, Gm10558, 2610012C04Rik, Gm4793, Gm38399, Hoxd10,
Meis2, Sptbn5, Dusp2, Astl, Adra2b*, Rxfp1*, Gm26984, Mex3a,
4731419109Rik, Gm23723, Gm17690, Cdc14a, Dkk2, Zfp593, Kif17,
Tmem51os1, Prkcz, Gm26825, Atg9b, Gm43852, Sgsm1, Cttnbp2,
Gm44389, Rpia, Elmod3, Retsat, Gm45053, Fbxo41, Gm15737, Ttll3,
Gm6375, Gm47692, Rnf225, Mamstr, Gm18210, Arhgef7, Actg-ps1,
Hsd11b2, Gm20416, Pgr, Gm36198, Gm16853, Gm8543, Gramd1b*,
Gm5366, Cd109, Gm39384, 4930553121 Rik, Gm26359, Eif4a1, Tbx4,
Vmp1*, Gm53, Hoxb9, Stat5a, Cacng4*, Dnah17, Nptx1, Actg1,
Gm6682, Greb1, Ror2*, Gm47586, Gm47603, Gm47585, Gm47602,
Gm16907, Thrb, Gm48321, Trim13, Gm7232, Gm8174, Osr2, Samd12,
1700015H07Rik, Gm27782, Apol8, Foxred2, Acr, Fgd4, AC163694.1,
Chodl, Adamts1, Bach1, Gm7831, Cacna1h*, Plin5, Sema6b*,
Pcdhgc4*, Gm29994, Neat1, Fads2*, Ddx3x, Gm5388, Tpt1-ps6, Xist

TABLE 107
Genes with higher expression level in both V-ECs
and NV-ECs in brain parenchyma compared to
other tissues (18 genes)
Gene name
Gm13324, Ccdc141, Gatm, Ciart, Gm11827, Tmeff1*,
Ak4, Tcf23, D630030B08Rik, Gm42466,
4930517G19Rik, Gm45828, Gm44981, Dnah3,
Gm48495, Gm9176, Pik3ip1*, Fam107a

TABLE 108
Genes with higher expression level in both V-ECs
and NV-ECs in choroid plexus compared to other
tissues (27 genes)
Gene name
1500015O10Rik, Kcnj13, Ralgps2, Slc4a10*, Col9a3,
Serpini1, Zfp697, Plppr4*, Steap4*, Slc29a4*,
Tmem72, Gm44891, Slc5a5*, Drc7, Vat1l,
Fam81a, Moxd1, Nsg2*, Sostdc1, Otx2,
2900040C04Rik, Lsamp, Kcne2*, Nrxn1*,
Ttr, Prr32, Frmpd4

TABLE 109
Genes with higher expression level in both V-ECs
and NV-ECs in pia matter compared to other
tissues (2 genes)
Gene name
Tbx1, 2810407A14Rik

TABLE 110
Genes with higher expression level in both V-ECs
and NV-ECs in dura matter compared to other
tissues (9 genes)
Gene name
Eya1, Kcnt2*, Prg4, Gm5552, Gm25535, Gm26802,
Nrxn3*, Gm17083, AC151299.1

TABLE 111
Genes with higher expression level in both V-ECs
and NV-ECs in peripheral lymph node compared
to other tissues (4 genes): cell surface
molecules only
Gene name
Marco, Abca13, F2r, Slc14a1

TABLE 112
Genes with higher expression level in both V-ECs
and NV-ECs in Peyer's patch compared to other
tissues (1 gene): cell surface molecules only
Gene name
Faim2

TABLE 113
Genes with higher expression level in both V-ECs
and NV-ECs in thymus compared to
other tissues (4 genes): cell surface
molecules only
Gene name
Lrmp, P2ry2, Kcng4, Lvrn

TABLE 114
Genes with higher expression level in both V-ECs
and NV-ECs in small intestine compared to
other tissues (15 genes): cell surface
molecules only
Gene name
Sctr, Lax1, Cftr, Vmn1r185, Slc7a9, Unc5d, Glipr1,
Tnfrsf13b, Tm4sf5, Cd180, II17rb, Adra1a,Tnfrsf17,
Tigit, Slc3a1

TABLE 115
Genes with higher expression level in both V-ECs
and NV-ECs in colon compared to other tissues
(3 genes): cell surface molecules only
Gene name
Il1rl1, Nmur1, Olfr558

TABLE 116
Genes with higher expression level in both V-ECs
and NV-ECs in subcutaneous adipose tissue
compared to other tissues (1 gene):
cell surface molecules only
Gene name
Pth1r

TABLE 117
Genes with higher expression level in ECs (both V-
ECs and NV-ECs) in bone tissue compared to
other tissues (7 genes): cell surface
molecules only Gene name
Rhd, Antxr2, Adam12, Mmgt2, Trpv3, Mmp14, Slc7a8

TABLE 118
Genes with higher expression level in ECs (both V-
ECs and NV-ECs) in uterus compared to other
tissues (10 genes): cell surface molecules only
Gene name
Adra2b, Rxfp1, Gramd1b, Vmp1, Cacng4, Ror2,
Cacna1h, Sema6b, Pcdhgc4, Fads2

TABLE 119
Genes with higher expression level in ECs (both V-
ECs and NV-ECs) in brain parenchyma compared to
other tissues (2 genes): cell surface molecules only
Gene name
Tmeff1, Pik3ip1

TABLE 120
Genes with higher expression level in ECs (both V-
ECs and NV-ECs) in choroid plexus compared to
other tissues (8 genes): cell surface
molecules only
Gene name
Slc4a10, Plppr4, Steap4, Slc29a4, Slc5a5, Nsg2,
Kcne2, Nrxn1

TABLE 121
Genes with higher expression level in ECs (both V-
ECs and NV-ECs) in dura matter compared to other
tissues (2 genes): cell surface molecules only
Gene name
Kcnt2, Nrxn3

### Example 3. Prospects of the Invention

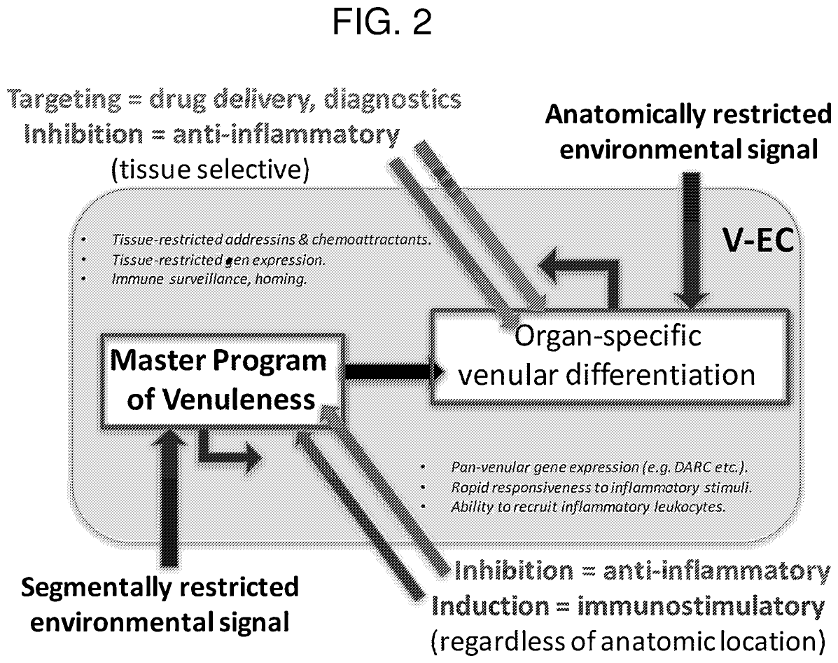

This invention describes the identification of candidate master regulators of global and tissue-specific V-EC and NV-EC phenotype. Lists of genes are identified that are selectively expressed in venules or non-venules (e.g., capillaries and arterioles) either globally or in a tissue-restricted fashion. The newly identified segmental differences in microvascular gene expression are relevant because only venules, but not other microvessels, have the capacity to recruit inflammatory leukocytes from blood into tissues. The molecular mechanisms that govern venular function have been unknown, but are likely to be rooted in venule-specific gene expression. Thus, this invention allows for: (a) screening of inhibitors of venular differentiation programs that may exert potent anti-inflammatory activity either systemically or in a tissue-restricted fashion; (b) identification of venule-restricted surface molecules that will allow specific targeting of therapeutic and/or diagnostic agents to venules or non-venules in select target tissues; and (c) screening of inducers of venular differentiation programs that are immunostimulatory in nature for onco-immunotherapy. The prospects of this invention are summarized in FIG. 2.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent or patent application was specifically and individually indicated to be incorporated by reference in its entirety.

What is claimed is:

1. A method of reducing venuleness of an endothelial cell (EC), comprising contacting the EC with an effective amount of:
(a) an agent that reduces expression of at least one gene from Tables 1-18 and/or Tables 36-52 exhibiting higher expression level in venule ECs (V-ECs) compared to non-venule ECs (NV-ECs); and/or
(b) an agent that increases expression of at least one gene from Tables 19-35 and/or Tables 53-68 exhibiting higher expression level in NV-ECs compared to V-ECs.

2. The method of claim 1, wherein venuleness of the EC is reduced in one or more tissues.

3. The method of claim 2, wherein the one or more tissues comprises peripheral lymph node, mesenteric lymph node, Peyer's patch, thymus, visceral adipose tissue, small intestine, colon, subcutaneous adipose tissue, bone tissue, uterus, brain parenchyma, choroid plexus, pia matter, or dura matter.

4. The method of claim 1, wherein the agent that reduces expression of at least one gene exhibiting higher expression level in V-ECs compared to NV-ECs is selected from one or more of a polypeptide, a small molecule, a nucleic acid molecule, a nuclease, a viral vector, or a plasmid, wherein:
(a) the polypeptide is an inhibitory antibody or antigen-binding fragment thereof directed to one or more of the genes from Tables 1-18 and/or Tables 36-52, wherein optionally, the antibody or antigen-binding fragment thereof is directed to one or more of the cell surface molecule genes from Tables 36-52;
(b) the small molecule is a small molecule inhibitor;
(c) the nucleic acid molecule is an inhibitory RNA molecule; wherein optionally, the inhibitory RNA molecule is a small interfering RNA (siRNA), a short hairpin RNA (shRNA), a microRNA (miRNA), a messenger RNA (mRNA), and/or a modified mRNA, wherein the mRNA and/or the modified mRNA encodes an antibody and/or a protein that is directed to one or more of the genes from Tables 1-18 and/or Tables 36-52;
(d) the nuclease is a Cas9, a transcription activator-like effector nuclease (TALEN), and/or a zinc-finger nuclease (ZFN);
(e) the viral vector is selected from the group consisting of an adeno-associated virus (AAV), an adenovirus, a parvovirus, a coronavirus, a rhabdovirus, a paramyxovirus, a picornavirus, an alphavirus, a herpes virus, a poxvirus, and a lentivirus; and
(f) the plasmid encodes an antibody, antigen-binding fragment, protein, and/or peptide that is directed to one or more of the genes from Tables 1-18 and/or Tables 36-52.

5. (canceled)

6. The method of claim 1, wherein the agent that increases expression of at least one gene exhibiting higher expression level in NV-ECs compared to V-ECs is selected from one or more of a polypeptide, a small molecule, a nucleic acid molecule, a viral vector, or a plasmid, wherein:
(a) the polypeptide is an activating antibody or antigen-binding fragment thereof directed to one or more of the genes from Tables 19-35 and/or Tables 53-68, wherein optionally, the activating antibody or antigen-binding fragment thereof is directed to one or more of the cell surface molecule genes from Tables 53-68;
(b) the small molecule is a small molecule activator;
(c) the nucleic acid molecule is an activating RNA molecule, wherein optionally, the activating RNA molecule is a small activating RNA (saRNA), or an mRNA;
(d) the viral vector is selected from the group consisting of an AAV, an adenovirus, a parvovirus, a coronavirus, a rhabdovirus, a paramyxovirus, a picornavirus, an alphavirus, a herpes virus, a poxvirus, and a lentivirus; and
(e) the plasmid is an overexpression plasmid and/or encodes an antibody, antigen-binding fragment, protein, and/or peptide that is directed to one of more of the genes from Tables 19-35 and/or Tables 53-68.

7. (canceled)

8. The method of claim 1, further comprising conjugating the agent to an EC targeting molecule, wherein the EC targeting molecule is directed to one or more genes from Tables 69-121 exhibiting high expression level in ECs.

9. The method of claim 8, wherein the EC targeting molecule is an antibody.

10. The method of claim 9, wherein the EC targeting molecule is an antibody that binds to one or more cell surface molecule genes listed in Tables 83-96, and/or Tables 111-121.

11-13. (canceled)

14. The method of claim 1, wherein the method reduces A inflammation in a tissue.

15. The method of claim 1, wherein the method treats an inflammatory disease in a subject.

16-18. (canceled)

19. The method of claim 15, wherein the inflammatory disease is selected from one or more of endotoxemia, sepsis, obesity-related insulin resistance, diabetes, polycystic ovary syndrome, metabolic syndrome, hypertension, cerebrovascular accident, myocardial infarction, congestive heart failure, cholecystitis, gout, osteoarthritis, Pickwickian syndrome, sleep apnea, atherosclerosis, inflammatory bowel disease, rheumatoid arthritis, vasculitis, transplant rejection, asthma, ischaemic heart disease, appendicitis, peptic, gastric and duodenal ulcers, peritonitis, pancreatitis, ulcerative, pseudomembranous, acute and ischemic colitis, diverticulitis, epiglottitis, achalasia, cholangitis, hepatitis, Crohn's disease, enteritis, Whipple's disease, allergy, anaphylactic shock, immune complex disease, organ ischemia, reperfusion injury, organ necrosis, hay fever, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, cystic fibrosis, pneumonitis, alveolitis, bronchiolitis, pharyngitis, pleurisy, sinusitis, a parasitic infection, a bacterial infection, a viral infection, an autoimmune disease, influenza, respiratory syncytial virus infection, herpes infection, HIV infection, hepatitis B virus infection, hepatitis C virus infection, disseminated bacteremia, Dengue fever, candidiasis, malaria, filariasis, amebiasis, hydatid cysts, burns, dermatitis, dermatomyositis, sunburn, urticaria, warts, wheals, vasulitis, angiitis, endocarditis, arteritis, thrombophlebitis, pericarditis, myocarditis, myocardial ischemia, periarteritis nodosa, rheumatic fever, celiac disease, adult respiratory distress syndrome, meningitis, encephalitis, cerebral infarction, cerebral embolism, Guillain-Barre syndrome, neuritis, neuralgia, spinal cord injury, paralysis, uveitis, arthritides, arthralgias, osteomyelitis, fasciitis, Paget's disease, periodontal disease, synovitis, myasthenia gravis, thyroiditis, systemic lupus erythematosus, Goodpasture's syndrome, Behcets's syndrome, allograft rejection, graft-versus-host disease, ankylosing spondylitis, Berger's disease, Retier's syndrome, or Hodgkin's disease.

20-21. (canceled)

22. A method of increasing venuleness of an EC, comprising contacting the EC with an effective amount of:
(a) an agent that increases expression of at least one gene from Tables 1-18 and/or Tables 36-52 exhibiting higher expression level in V-ECs compared to NV-ECs; and/or
(b) an agent that decreases expression of at least one gene from Tables 19-35 and/or Tables 53-68 exhibiting higher expression level in NV-ECs compared to V-ECs.

23-24. (canceled)

25. The method of claim 22, wherein the agent that increases expression of at least one gene exhibiting higher expression level in V-ECs compared to NV-ECs is selected from one or more of a polypeptide, a small molecule, a nucleic acid molecule, a viral vector, or a plasmid, wherein:
(a) the polypeptide is an activating antibody or antigen-binding fragment thereof directed to one or more of the genes from Tables 1-18 and/or Tables 36-52, wherein optionally, the antibody or antigen-binding fragment thereof is directed to one or more of the cell surface molecule genes from Tables 36-52;
(b) the small molecule is a small molecule activator;
(c) the nucleic acid molecule is an activating RNA molecule, wherein optionally, the activating RNA molecule is an saRNA, or an mRNA;
(d) the viral vector is selected from the group consisting of an AAV, an adenovirus, a parvovirus, a coronavirus, a rhabdovirus, a paramyxovirus, a picornavirus, an alphavirus, a herpes virus, a poxvirus, and a lentivirus; and
(e) the plasmid is an overexpression plasmid and/or encodes an antibody, antigen-binding fragment, protein, and/or peptide that is directed to one of more of the genes from Tables 1-18 and/or Tables 36-52.

26. (canceled)

27. The method of claim 22, wherein the agent that reduces expression of at least one gene exhibiting higher expression level in NV-ECs compared to V-ECs is selected from one or more of a polypeptide, a small molecule, a nucleic acid molecule, a nuclease, a viral vector, or a plasmid, wherein:
(a) the polypeptide is an inhibitory antibody or antigen-binding fragment thereof directed to one or more of the genes from Tables 19-35 and/or Tables 53-68, wherein optionally, the inhibitory antibody or antigen-binding fragment is directed to one or more of the cell surface molecule genes from Tables 53-68;
(b) the small molecule is a small molecule inhibitor;
(c) the nucleic acid molecule is an inhibitory RNA molecule, wherein optionally, the inhibitory RNA molecule is an siRNA, a shRNA, a miRNA: a mRNA and/or a modified mRNA, wherein the mRNA and/or the modified mRNA encodes an antibody and/or a protein that is directed to one or more of the genes from Tables 19-35 and/or Tables 53-68;
(d) the nuclease is a Cas9, a TALEN, and/or a ZFN;
(e) the viral vector is selected from the group consisting of an AAV, an adenovirus, a parvovirus, a coronavirus, a rhabdovirus, a paramyxovirus, a picornavirus, an alphavirus, a herpes virus, a poxvirus, and a lentivirus; and
(f) the plasmid encodes an antibody, antigen-binding fragment, protein, and/or peptide that is directed to one or more of the genes from Tables 19-35 and/or Tables 53-68.

28-34. (canceled)

35. The method of claim 22, wherein the method increases leukocyte recruitment in a tissue.

36. The method of claim 22, wherein the method treats A cancer in a subject.

37-40. (canceled)

41. The method of claim 36, wherein the cancer is selected from one or more of a cancer of the nervous system, uterine cancer, endometrial cancer multiple myeloma, breast cancer, acute myelogenous leukemia, acute lymphoblastic leukemia, chronic lymphocytic leukemia, myelodysplastic syndrome, chronic myelogenous leukemia—chronic phase, diffuse large B-cell lymphoma, cutaneous T-cell lymphoma, peripheral T-cell lymphoma, Hodgkin's lymphoma, hepatocellular carcinoma, cervical cancer, prostate cancer, kidney cancer, renal cell carcinoma, esophageal cancer, melanoma, glioma, brain cancer, astrocytoma, oligoastrocytoma, brainstem glioma, ependymoma, retinoblastoma, brain metastasis, ganglioneuroma, Pacinian neuroma, acoustic neuroma, pancreatic cancer, ovarian cancer, gastrointestinal stromal tumors, sarcoma, estrogen receptor-positive breast cancer, lung cancer, non-small cell lung carcinoma, mesothelioma, intestinal cancer, colon cancer, bladder cancer, adrenal cancer, gallbladder cancer, or squamous cell carcinoma of the head and neck.

42. The method of claim 36, further comprising administration of one or more anti-cancer therapeutics to the subject.

43. The method of claim 42, wherein the anti-cancer therapeutic:
(a) is selected from one or more of a chemotherapeutic, a targeted anti-cancer therapeutic, or an anti-cancer immunotherapeutic; and/or
(b) is conjugated to one or more EC targeting molecule.

44. The method of claim 43, wherein:
(a) the chemotherapeutic is selected from one or more of an anthracycline, a nucleoside analog, a platinum-based anti-neoplastic agent, a taxane, a vinca alkaloid, a glycopeptide antibiotic, or a polypeptide antibiotic;
(b) the targeted anti-cancer therapeutic is selected from one or more of a tyrosine kinase inhibitor, a PI3K inhibitor, a multi-kinase inhibitor, a CDK4/6 inhibitor, an mTOR inhibitor, a NOTCH inhibitor, an HSP90 inhibitor, an HSP70 inhibitor, a proteasome inhibitor, or a tumor metabolism inhibitor; and/or
(c) the anti-cancer immunotherapeutic is selected from one or more of a cytokine, a monoclonal antibody, an immune checkpoint inhibitor, an inhibitor of the Carma1-BCL10-MALT-1 complex, a cancer vaccine, a tumor-infiltrating lymphocyte, a CAR T-cell, or a non-specific immunotherapeutic.
